(19) United States
(12) Patent Application Publication
(10) Pub. No.: US 2023/0051840 A1
(43) Pub. Date: Feb. 16, 2023

(54) DEVICES AND METHODS FOR TRANSFECTION AND FOR GENERATION OF CLONAL POPULATIONS OF CELLS

(71) Applicant: Synthego Corporation, Redwood City, CA (US)

(72) Inventors: Reed Kelso, San Francisco, CA (US); Paul Dabrowski, San Francisco, CA (US); Ivan Razinkov, Redwood City, VA (US); Alexander Brown, Redwood City, CA (US); Brandon Whitney, Redwood City, CA (US); Maryam Khosroshahi, Redwood City, CA (US)

(21) Appl. No.: 17/793,861

(22) PCT Filed: Jan. 20, 2021
(86) PCT No.: PCT/US2021/014218

§ 371 Date: Jul. 19, 2022

Related U.S. Application Data
(60) Provisional application No. 62/963,689, filed on Jan. 21, 2020.

(51) Int. Cl. C12M 1/00; C12M 1/36

(52) U.S. Cl. CPC C12M 29/10; C12M 41/48; C12N 5/0663

(57) ABSTRACT

Disclosed herein are cartridges for transfecting cells and/or generating clonal populations of cells comprising: a) a first compartment configured for performing cell transfection, wherein the first compartment comprises a first inlet configured for introduction of a cell sample; b) a second compartment configured for performing cell selection, wherein an inlet of the second compartment is operably coupled to an outlet of the first compartment, and wherein the second compartment further comprises at least one optically-transparent wall and an outlet that is operably coupled to an intermediate cell removal port; and c) a third compartment configured for performing cell expansion, wherein an inlet of the third compartment is operably coupled to the outlet of the second compartment.

### CROSS-REFERENCE

This application claims priority to U.S. provisional patent application No. 62/963,689 filed Jan. 21, 2020, which is herein incorporated by reference in its entirety.

### BACKGROUND

Genome editing technologies are excellent tools for introducing precise, targeted alterations in a genome, however, the problems of carrying out such editing and generating clonal populations of edited cells in a high throughput fashion still exist. The present disclosure addresses, among other things, these and other related problems that exist in the current field of genome editing technology.

### SUMMARY

Devices, methods, and systems for transfecting cells and/or generating clonal populations of cells are described.

Disclosed herein are cartridges comprising: a) a first compartment configured for performing cell transfection, wherein the first compartment comprises a first inlet configured for introduction of a cell sample; b) a second compartment configured for performing cell selection, wherein an inlet of the second compartment is operably coupled to an outlet of the first compartment, and wherein the second compartment further comprises at least one optically-transparent wall and an outlet that is operably coupled to an intermediate cell removal port; and c) a third compartment configured for performing cell expansion, wherein an inlet of the third compartment is operably coupled to the outlet of the second compartment.

Also disclosed are cartridges comprising: a) a first compartment configured for performing cell transfection, wherein the first compartment comprises a first inlet configured for introduction of a cell sample; b) a second compartment configured for performing cell selection, wherein an inlet of the second compartment is operably coupled to an outlet of the first compartment, and wherein the second compartment further comprises at least one optically-transparent wall; and c) a third compartment configured for performing cell expansion, wherein the third compartment comprises at least one pair of electrodes configured for performing electrical impedance measurements, and wherein an inlet of the third compartment is operably coupled to the outlet of the second compartment.

Also disclosed are cartridges comprising: a) a first compartment configured for performing cell transfection, wherein the first compartment comprises a first inlet configured for introduction of a cell sample; b) a second compartment configured for performing cell selection, wherein an inlet of the second compartment is operably coupled to an outlet of the first compartment, and wherein the second compartment further comprises at least one optically-transparent wall that is operably coupled to a source of laser light for performing photoablation and photodetachment; and c) a third compartment configured for performing cell expansion, wherein an inlet of the third compartment is operably coupled to the outlet of the second fluid compartment.

Also disclosed are cartridges comprising: a) at least one compartment configured for performing cell transfection, cell selection, cell expansion, or any combination thereof, wherein the cartridge comprises an inlet configured for introduction of a cell sample and the at least one compartment comprises an optically-transparent wall operably couplable to a light source to facilitate performance of both a photodetachment process and a photoablation process.

In some embodiments of the disclosed cartridges, the first compartment (or at least one compartment) further comprises at least one of: (i) a second inlet configured for introduction of a transfection agent, (ii) a constricted flow path, (iii) a pair of electrodes in electrical contact with and positioned on opposing surfaces of the first compartment or at least one compartment, and (iv) an optically-transparent wall. In some embodiments, a longest dimension of the first compartment (or of at least one compartment) is between about 1 millimeter and about 30 millimeters. In some embodiments, a volume of the first compartment (or of at least one compartment) is between about 1 microliter and about 1 milliliter. In some embodiments, the constricted flow path comprises a constriction in at least one dimension that ranges from about 2 micrometers to about 10 micrometers in width. In some embodiments, the constricted flow path comprises a constriction in at least one dimension that is smaller than the average diameter of a cell of the cell sample. In some embodiments, the constricted flow path comprises a constriction in at least one dimension that is smaller than one half of the average diameter of a cell of the cell sample. In some embodiments, the pair of electrodes comprise parallel plate electrodes. In some embodiments, the pair of electrodes are fabricated from platinum, gold, silver, copper, zinc, aluminum, graphene, or indium tin oxide. In some embodiments, the pair of electrodes are separated by a distance ranging from about 10 micrometers to about 10 millimeters. In some embodiments, the optically-transparent wall of the first compartment (or of at least one compartment) is transparent in the ultraviolet, visible, or near-infrared regions of the electromagnetic spectrum, or any combination thereof. In some embodiments, the optically-transparent wall of the first compartment (or of at least one compartment) is transparent in a wavelength range centered at about 355 nm. In some embodiments, the optically-transparent wall of the first compartment (or of at least one compartment) is transparent in a wavelength range centered at about 785 nm. In some embodiments, the optically-transparent wall of the first compartment (or of at least one compartment) is transparent in the range from about 1440 nm to about 1450 nm. In some embodiments, a longest dimension of the second compartment (or of at least one compartment) is between about 1 centimeter and about 10 centimeters. In some embodiments, a volume of the second compartment (or of at least one compartment) is between about 1 microliter and about 10 milliliters. In some embodiments, the optically-transparent wall of the second compartment (or of at least one compartment) is transparent in the ultraviolet, visible, or near-infrared regions of the electromagnetic spectrum, or any combination thereof. In some embodiments, the optically-transparent wall of the second compartment (or of at least one compartment) is transparent in the range from about 1440 nm to about 1450 nm. In some embodiments, a wall of the second compartment (or of at least one compartment) comprises a surface coating or surface treatment to facilitate attachment of adherent cells. In some embodiments, a wall of the second compartment (or of at least one compartment) comprises a surface coating or surface treatment to facilitate attachment of suspension cells. In some embodiments, the surface coating is selected from the group consisting of an α-poly-lysine coating, a collagen coating, a poly-1-ornithine, a fibronectin coating, a laminin coating, a Synthemax™ vitronectin coating, an iMatrix-511 recombinant laminin coating, and any combination thereof. In some embodiments, the surface treatment comprises a plasma treatment, a UV treatment, an ozone treatment, or any combination thereof. In some embodiments, the wall of the second compartment (or of at least one compartment) that comprises the surface coating or surface treatment is the optically-transparent wall. In some embodiments, the second compartment (or at least one compartment) comprises a chamber having no physical barriers, flow constrictions, or partitions positioned therein. In some embodiments, a longest dimension of the third compartment (or at least one compartment) is between about 1 centimeter and about 20 centimeters. In some embodiments, a volume of the third compartment (or at least one compartment) is between about 1 microliter and about 1 milliliter. In some embodiments, the third compartment (or at least one compartment) further comprises at least one optically-transparent wall. In some embodiments, the optically-transparent wall is transparent in the ultraviolet, visible, or near-infrared regions of the electromagnetic spectrum, or any combination thereof. In some embodiments, the third compartment (or at least one compartment) further comprises at least one pair of electrodes configured for performing electrical impedance measurements. In some embodiments, the cartridge further comprises a fourth compartment (or at least one compartment) configured for storing a cell growth medium. In some embodiments, the cartridge further comprises a fifth compartment (or at least one compartment) configured for storing waste. In some embodiments, the fourth or fifth compartment (or at least one compartment) further comprises a gas permeable membrane. In some embodiments, the inlet of the second compartment (or at least one compartment) is operably coupled to a source of a reagent that facilitates detachment of cells from a surface within the second compartment (or the at least one compartment). In some embodiments, the inlet of the third compartment (or at least one compartment) is operably coupled to a source of a reagent that facilitates detachment of cells from a surface within the third compartment (or an at least second compartment). In some embodiments, the cartridge is fabricated from glass, fused-silica, silicon, polydimethylsiloxane (PDMS), polymethylmethacrylate (PMMA), polycarbonate (PC), polypropylene (PP), polyethylene (PE), high density polyethylene (HDPE), polyimide (PI), cyclic olefin polymers (COP), cyclic olefin copolymers (COC), polyethylene terephthalate (PET), polystyrene (PS), epoxy resin, ceramic, metal, flexdym or any combination thereof. In some embodiments, the outlet of the second compartment (or at least one compartment) is operably coupled to the cell removal port and the inlet of the third compartment (or an at least second compartment) using a three-way valve. In some embodiments, the inlet of the third compartment (or at least one compartment) is operably coupled to the outlet of the second compartment (or an at least second compartment) and the outlet of the fourth compartment (or an at least third compartment) using a three-way valve. In some embodiments, the three-way valve is a programmable three-way valve. In some embodiments, the microfluidic cartridge has a footprint that complies with American National Standards Institute (ANSI) Standard Number SLAS 4-2004 (R2012). In some embodiments, the microfluidic cartridge has a footprint that is 127.76 mm±0.5 mm in length and 85.48 mm±0.5 mm in width.

Disclosed herein are methods for producing a clonal population of transfected cells, the methods comprising: a) providing a cartridge, wherein the cartridge comprises at least one compartment configured for performing cell transfection, cell selection, cell expansion, or any combination thereof, and wherein at least one compartment comprises an optically-transparent wall; b) introducing a cell sample into the at least one compartment; c) transfecting the cell sample with one or more transfection agents; d) selecting at least one clonal cell colony derived from the transfected cell sample; e) performing photoablation to destroy all clonal cell colonies except the at least one clonal cell colony selected in (d); and subjecting the at least one clonal cell colony selected in (d) to one or more cycles of cell division and growth to produce a clonal population of transfected cells.

In some embodiments of the disclosed methods, the method may further comprise detaching a first subset of cells from the at least one clonal cell colony selected in (d) and removing them from the cartridge for testing. In some embodiments, the results of said testing, e.g., sequencing of clones to confirm the sequence of a desired edit, may be used as the basis for selecting cells for clonal expansion. In some embodiments, the method may further comprise performing photoablation to destroy all remaining clonal cell colonies except a subset of those for which a first subset of cells was detached and subjected to testing. In some embodiments, the cell sample comprises adherent cells. In some embodiments, the cell sample comprises suspension cells. In some embodiments, the cell sample comprises mammalian cells. In some embodiments, the mammalian cells are human cells. In some embodiments, the number of cells in the cell sample is less than 10,000. In some embodiments, the number of cells in the cell sample is less than 5,000. In some embodiments, the number of cells in the cell sample is less than 1,000. In some embodiments, the number of cells in the cell sample is less than 500. In some embodiments, the one or more transfection agents comprise one or more types of DNA molecule, RNA molecule, oligonucleotide, aptamer, non-plasmid nucleic acid molecule, ribonucleoprotein (RNP), plasmid, viral vector, cosmid, artificial chromosome, or any combination thereof. In some embodiments, the transfecting performed in (c) comprises chemical transfection, mechanical transfection (squeezing), electroporation, laser-induced photoporation, needle-based poration, impalefection, magnetofection, sonoporation, or any combination thereof. In some embodiments, the clonal cell colonies derived from the transfected cell sample are grown by seeding a surface of at least one compartment with transfected cells at a cell surface density of less than or equal to 50 cells/mm². In some embodiments, the clonal cell colonies derived from the transfected cell sample are grown by seeding a surface of at least one compartment with transfected cells at a cell surface density of less than or equal to 10 cells/mm². In some embodiments, the clonal cell colonies derived from the transfected cell sample are grown by seeding a surface of at least one compartment with transfected cells at a cell surface density of less than or equal to 5 cells/mm². In some embodiments, after seeding at least one compartment with transfected cells, any clusters of cells comprising two or more cells are destroyed using a photoablation step prior to allowing single cells to form clonal colonies. In some embodiments, the selecting in (d) comprises randomly-selecting one or more clonal cell colonies. In some embodiments, the selecting in (d) comprises selecting the at least one clonal cell colony based on a position on an interior surface of the at least one compartment. In some embodiments, the selecting in (d) is based on a number of cells within the at least one clonal cell colony, a morphology of cells within the at least one clonal cell colony, a surface density of cells within the at least one clonal cell colony, a growth pattern of cells within the at least one clonal cell colony, a growth rate of cells within the at least one clonal cell colony, a division rate of cells within the at least one clonal cell colony, expression of an exogenous reporter by cells within the at least one clonal cell colony, or any combination thereof. In some embodiments, the selecting in (d) is based on imaging a surface on which, or a volume within which, the at least one clonal cell colony is grown. In some embodiments, the imaging comprises performing bright-field imaging, dark-field imaging, phase contrast imaging, fluorescence imaging, or any combination thereof. In some embodiments, acquired images are processed using automated image analysis software. In some embodiments, a field-of-view of an imaging system used to perform the imaging is smaller than an area of the surface or volume, and wherein the imaging comprises acquiring two or more individual images that collectively cover all or a portion of the area of the surface or volume. In some embodiments, the imaging is performed at a frequency of at least once per day. In some embodiments, the imaging is performed at a frequency of at least once per hour. In some embodiments, the selecting in (d) is performed automatically based on automated image analysis of one or more images. In some embodiments, a wall of at least one compartment comprises a surface coating or surface treatment to facilitate attachment of adherent cells. In some embodiments, a wall of at least one compartment comprises a surface coating or surface treatment to facilitate attachment of suspension cells. In some embodiments, the surface coating is selected from the group consisting of an α-poly-lysine coating, a collagen coating, a poly-1-ornithine, a fibronectin coating, a laminin coating, a Synthemax™ vitronectin coating, an iMatrix-511 recombinant laminin coating, and any combination thereof. In some embodiments, the surface treatment comprises a plasma treatment, a UV treatment, an ozone treatment, or any combination thereof. In some embodiments, the wall of the at least one compartment that comprises the surface coating or surface treatment is the optically-transparent wall. In some embodiments, the first subset of cells is detached using laser photodetachment. In some embodiments, the method further comprises subjecting the first subset of cells to a flow of liquid directed across a surface on which the at least one clonal cell colony is grown while a region of the surface beneath or adjacent to the at least one clonal cell colony is illuminated with laser light. In some embodiments, illumination with laser light results in cleavage of a photocleavable linker used to tether cells to the wall of the at least one compartment. In some embodiments, illumination with laser light results in a photothermal detachment of the first subset of cells. In some embodiments, illumination with laser light results in a photomechanical detachment of the one or more selected cells. In some embodiments, illumination with laser light results in a photoacoustic detachment of the one or more selected cells. In some embodiments, the laser photodetachment is performed using laser light in a wavelength range of about 1440 nm to about 1450 nm. In some embodiments, an efficiency of photodetaching the first subset of cells is at least 80%. In some embodiments, an efficiency of photodetaching the first subset of cells is at least 90%. In some embodiments, an efficiency of photodetaching the first subset of cells is at least 95%. In some embodiments, the first subset of cells comprises fewer than 100 cells. In some embodiments, the first subset of cells comprises fewer than 50 cells. In some embodiments, the first subset of cells comprises fewer than 10 cells. In some embodiments, the first subset of cells comprises a single cell. In some embodiments, the testing comprises nucleic acid sequencing. In some embodiments, the testing comprises gene expression profiling. In some embodiments, the testing comprises detection of a modified gene. In some embodiments, the testing comprises detection of a CRISPR edited gene. In some embodiments, the testing comprises performing a restriction site analysis of nucleic acid molecules. In some embodiments, the testing comprises detection of a protein. In some embodiments, the protein comprises a mutant protein, a reporter protein, or a genetically-engineered protein. In some embodiments, the testing comprises detection of a change in an intracellular signaling pathway due to an altered protein function. In some embodiments, the photoablation is performed using laser light in a wavelength range of about 1440 nm to about 1450 nm. In some embodiments, an efficiency of photoablation is at least 80%. In some embodiments, an efficiency of photoablation is at least 90%. In some embodiments, an efficiency of photoablation is at least 95%. In some embodiments, growth of the clonal population of transfected cells is monitored using electrical impedance measurements. In some embodiments, the method further comprises harvesting the clonal population of transfected cells after a specified number of cell division and growth cycles. In some embodiments, the method further comprises harvesting the clonal population of transfected cells after they have reached at least 70% confluency in the at least one compartment.

Disclosed herein are apparatus comprising: a) a cartridge, wherein the cartridge comprises at least one compartment configured for performing cell transfection, cell selection, cell expansion, or any combination thereof, wherein at least one compartment comprises an optically-transparent wall that is operably coupled to a source of laser light for performing photoablation and photodetachment; and b) a controller.

In some embodiments, the controller is configured to perform at least one of: i) controlling timing and flowrate for one or more fluids flowing through the cartridge; ii) performing manual, semi-automated, or fully-automated image processing of images acquired by an imaging unit and, based on data derived from the processed images, selecting a first subset of cells for laser-based photodetachment and a second subset of cells for laser-based photoablation; and iii) controlling laser operating parameters for one or more lasers and a laser targeting unit such that the first subset of cells is photodetached and the second subset of cells is photoablated. In some embodiments, the first subset of cells and the second subset of cells are both derived from a single clonal cell colony. In some embodiments, the laser targeting unit comprises a translation stage configured to accurately position cells growing on a surface within, or within a volume of, the at least one compartment at, or adjacent to, a laser focal point on an object plane of the imaging unit. In some embodiments, the laser targeting unit comprises a scanning mechanism configured to direct focused laser light at, or adjacent to, the positions of one or more cells growing on a surface within, or within a volume of, the at least one compartment. In some embodiments, cell transfection is performed in a first compartment, and cell selection and cell expansion are performed in a second compartment. In some embodiments, cell transfection, cell selection, and cell expansion are each performed in a separate compartment. In some embodiments, cell transfection, cell selection, and cell expansion are all performed in the same compartment. In some embodiments, the imaging unit is configured to perform bright-field imaging, dark-field imaging, phase contrast imaging, fluorescence imaging, or any combination thereof. In some embodiments, a field-of-view of the imaging unit is smaller than an area of a surface of, or volume within, the at least one compartment on or within which cells are grown or attached, and wherein the imaging unit is configured to acquire and tile two or more individual images that collectively cover all or a portion of the area of the surface or volume. In some embodiments, the imaging unit is configured to acquire images at a frequency of at least once per day. In some embodiments, the imaging unit is configured to acquire images at a frequency of at least once per hour. In some embodiments, the selecting in (ii) comprises randomly-selecting one or more clonal cell colonies. In some embodiments, the selecting in (ii) comprises selecting one or more clonal cell colonies based on a position on a surface of the at least one compartment. In some embodiments, the selecting in (ii) is based on a number of cells within a clonal cell colony, a morphology of cells within a clonal cell colony, a surface density of cells within a clonal cell colony, a growth pattern of cells within a clonal cell colony, a growth rate of cells within a clonal cell colony, a division rate of cells within a clonal cell colony, expression of an exogenous reporter by cells within a clonal cell colony, or any combination thereof. In some embodiments, the same laser is used to perform photoablation and photodetachment. In some embodiments, the one or more lasers used for photodetachment and photoablation are optically coupled to the imaging system through an objective lens used for imaging. In some embodiments, the one or more lasers used to perform photodetachment and photoablation comprise at least one pulsed laser. In some embodiments, the one or more lasers used to perform photodetachment and photoablation comprise at least one infrared laser. In some embodiments, the apparatus is operably switched between a photodetachment operating mode and a photoablation operating mode by controlling a laser spot size, a laser spot shape, a laser light intensity, a laser pulse frequency, a laser pulse energy, a total number of laser pulses delivered at a specified position on the surface or within the volume of the at least one compartment, a position of a laser focal point relative to the surface or within the volume of the at least one compartment, or any combination thereof. In some embodiments, the controller is further configured to subject the first subset of cells to a flow of liquid directed across the surface within the at least one compartment while a region of the surface beneath or adjacent to the first subset of cells is illuminated with laser light. In some embodiments, an efficiency of photodetaching the first subset of cells is at least 80%. In some embodiments, an efficiency of photodetaching the first subset of cells is at least 90%. In some embodiments, an efficiency of photodetaching the first subset of cells is at least 95%. In some embodiments, the second subset of cells is photoablated with an efficiency of greater than 90%. In some embodiments, the second subset of cells is photoablated with an efficiency of greater than 95%. In some embodiments, the second subset of cells is photoablated with an efficiency of greater than 99%. In some embodiments, the second subset of cells is photoablated with an efficiency of greater than 99.9%. In some embodiments, the one or more lasers are further configured to perform laser-based photoporation of cells in the at least one compartment. In some embodiments, at least one compartment of the cartridge is configured to perform chemical transfection, mechanical transfection (squeezing), electroporation, laser-induced photoporation, needle-based poration, impalefection, magnetofection, sonoporation, or any combination thereof. In some embodiments, the apparatus further comprises an incubator unit for maintaining the at least one compartment of the cartridge under a specified set of growth conditions.

Disclosed herein are non-transitory computer-readable media storing a set of instructions which, when executed by a processor, cause a processor-controlled system to perform steps comprising: a) controlling timing and flowrate for one or more fluids flowing through a cartridge comprising at least one compartment configured to perform cell transfection, cell selection, cell expansion, or any combination thereof; b) performing image processing of images acquired by an imaging unit configured to image a surface or volume within the at least one compartment and, based on data derived from the processed images, selecting: (i) a first subset of cells growing on a surface of or in a volume within the at least one compartment for laser-based photodetachment and (ii) a second subset of cells growing on a surface of or in a volume within the at least one compartment for laser-based photoablation; and c) controlling one or more operating parameters of one or more lasers and a laser targeting unit such that the first subset of cells is photodetached and the second subset of cells is photoablated.

In some embodiments, the selecting in (b) comprises randomly-selecting one or more clonal cell colonies. In some embodiments, the selecting in (b) comprises selecting one or more clonal cell colonies based on a position on an interior surface of the cell selection compartment. In some embodiments, the selecting in (b) is based on a number of cells within a clonal cell colony, a morphology of cells within a clonal cell colony, a surface density of cells within a clonal cell colony, a growth pattern of cells within a clonal cell colony, a growth rate of cells within a clonal cell colony, a division rate of cells within a clonal cell colony, expression of an exogenous reporter by cells within a clonal cell colony, or any combination thereof. In some embodiments, the processor-controlled system is operably switched between a photodetachment operating mode and a photoablation operating mode by: controlling a laser spot size, a laser spot shape, a laser light intensity, a laser pulse frequency, a laser pulse energy, a total number of laser pulses delivered at a specified position on a surface or within the volume of the at least one compartment, a position of a laser focal point relative to the surface within the at least one compartment, a position of a laser focal point within the volume of the at least one compartment, or any combination thereof. In some embodiments, the non-transitory computer-readable medium further comprises instructions for delivering the photodetached first subset of cells to an outlet port of the cartridge for testing. In some embodiments, the non-transitory computer-readable medium further comprises instructions for performing photodetachment of a third subset of cells following photoablation of the second subset of cells and delivering the detached third subset of cells to an at least second compartment configured to perform cell expansion.

### INCORPORATION BY REFERENCE

All publications, patents, and patent applications mentioned in this specification are herein incorporated by reference in their entirety to the same extent as if each individual publication, patent, or patent application was specifically and individually indicated to be incorporated by reference in its entirety. In the event of a conflict between a term herein and a term in an incorporated reference, the term herein controls.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 provides a non-limiting example of the layout of a cell transfection, selection, and clonal expansion cartridge of the present disclosure.

FIG. 2 provides a non-limiting illustration of the layout of fluid inlets, fluid outlets, fluid channels and fluid compartments (e.g., for cell transfection, selection, and growth, as well as for storage of culture medium and waste) in one instance of the disclosed cartridges.

FIGS. 3A-3D provide views of a partially-assembled and fully-assembled cell transfection, selection, and clonal expansion cartridge of the present disclosure. FIG. 3A provides a photograph of an injection-molded base plate of the cartridge. FIG. 3B provides an illustration of the cartridge comprising the base plate and attached cell selection chamber, cell expansion chamber, growth medium reservoir, waste reservoir, and valve. FIG. 3C provides a photograph of a prototype cartridge. FIG. 3D provides a photograph of the assembled cartridge.

FIG. 4 provides a non-limiting example of the process steps performed within a cell transfection, selection, and clonal expansion cartridge of the present disclosure.

FIGS. 5A-5E illustrate the use of laser photodetachment to selectively detach cells from a substrate on which they are grown. FIG. 5A: micrograph of cells on a growth surface within the cell selection compartment. FIG. 5B: micrograph of the same surface after selectively detaching cells. FIG. 5C: illustration of the selective detachment and removal of a selected subset of cells within a clonal cell cluster by irradiation with laser light. FIG. 5D: illustration of progressive detachment of the cells as the laser light is scanned along the surface underlying the selected cells. FIG. 5E: illustration of the further progressive detachment of the cells as the laser light continues to be scanned along the surface underlying the cells.

FIGS. 6A-6E illustrate the use of laser photodetachment in combination with directed fluid flow to selectively detach and remove cells from a substrate on which they are grown. FIG. 6A: micrograph of cells on a growth surface within the cell selection compartment. FIG. 6B: micrograph of the same surface after selectively detaching cells. FIG. 6C: illustration of the selective detachment and removal of a selected subset of cells within a clonal cell cluster by irradiation with laser light. FIG. 6D: illustration of progressive detachment of the cells as the laser light is scanned along the surface underlying the selected cells while a flow of fluid is directed across the surface. FIG. 6E: illustration of the further progressive detachment of the cells as the laser light continues to be scanned along the surface underlying the cells while a flow of fluid is directed across the surface.

FIG. 7 provides a non-limiting example of a block diagram for the software used to control a system for generation of clonal cell populations according to one aspect of the present disclosure.

FIGS. 8A-8F show clonal isolation of cells using photoablation and photodetachment in cartridges described herein. FIG. 8A: mixed populations of HEK293-GFP and RFP cells are shown after attachment on cartridges described herein. FIG. 8B: mixed populations of HEK293-GFP and RFP cells are shown after laser ablation. FIG. 8C: mixed populations of HEK293-GFP and RFP cells are shown after removal of dead cells by media flow, wherein the boxes indicate areas that were targeted for ablation. FIG. 8D: clonal HEK293-RFP colonies after photodetaching them from the cartridges are shown; no detectable cross contamination was observed after export. FIG. 8E: clonal HEK-GFP colonies after photodetaching them from the cartridges are shown; no detectable cross contamination was observed after export. FIG. 8F: non-clonal cross contaminated colonies containing both populations of cells are shown.

FIGS. 9A-9C depict testing cross-contamination via rotary valves of the cartridges described herein. FIG. 9A: the rotary valves and liquid paths of the cartridge are shown. Inoculated bacteria and sterile media were transported between valves using a common liquid path. FIG. 9B: shows no cross contamination was observed in the cartridge comprising rotary valves. Sterile media was deposited in column 1 of the microwell plate (Sterile), bacteria inoculated media was deposited in column 2 (Bacteria). Media in column 10 (+ CTRL) was inoculated by a pipette tip previously dipped in source wells, media in column 11 (− CTRL) was deposited directly on the plate and did not pass through rotary valves, column 12 (source) contains bacteria source media. FIG. 9C: provides a tabular depiction of the cross-contamination testing results showing that no cross contamination was observed in the cartridge comprising rotary valves.

FIGS. 10A-10B show various embodiments of the cartridges described herein as well as testing results of these various embodiments. FIG. 10A: multi-chamber embodiments of the cartridges described herein are shown. FIG. 10B: provides a table detailing the dimensions of the multi-chamber embodiments of the cartridges described herein and results of flow testing in said chambers.

FIGS. 11A-11B show various embodiments of the cartridges described herein. FIG. 11A: multi-chamber embodiments of the cartridges described herein are shown. FIG. 11B: provides a table detailing the dimensions of the multi-chamber embodiments of the cartridges described herein

FIG. 12 shows an embodiment of the cartridges described herein featuring 96 parallel miniature cell culture chambers, 6 larger cell culture chambers and fluidic connections.

FIG. 13 shows an embodiment of the cartridges described herein featuring two sterilize in place (SIP) systems to facilitate flow in and out of the cartridge.

FIG. 14 shows an embodiment of a media cartridge featuring a media filled syringe, a pump interface for filling the syringe and a SIP system.

### DETAILED DESCRIPTION

Methods, devices, and systems for performing cell transfection, selection, and clonal expansion to generate clonal cell populations of a defined genotype are described. Methods and systems for creating homogeneous clonal cell populations have become increasingly important for a variety of emerging applications including, but not limited to, expression and purification of genetically-engineered proteins, nucleic acids, and other cellular components; production of biologic drugs (biologics); and therapeutic applications of stem cells.

Existing methods for generating clonal populations of cells focus primarily on the use of serial dilution techniques and/or microfluidic devices to deposit a single cell in a culture plate well or other container and subsequently incubating it under appropriate conditions to ensure that it divides and develops into a mature clonal population. A challenge with the former is that random deposition of a cell suspension that is dilute enough to ensure that, on average, each culture plate well contains only a single cell also ensures that many of the culture plate wells will be empty (as is predicted by the Poisson distribution that governs random processes). Thus, this approach leads to a very inefficient process in terms of the number of culture plate wells that must be processed, and that also requires subsequent characterization of the population in each well to ensure that it does indeed contain a cell culture that arose from a single cell. Alternatively, microfluidic device-based approaches to depositing single cells are often prone to clogging and can subject the cells to mechanical stress that can negatively impact their viability.

Thus, there remains an unmet need for new technologies that provide a means for fast, efficient processing of cells and culture plates to produce clonal cell populations at a commercial scale. The microfluidic devices (or cartridges) disclosed herein provide for integrated cell processing functionality packaged in a compact format that, in some instances, are disposable and/or compatible for use with standard laboratory automation equipment. The functional components of the devices or cartridges may comprise: a cell transfection compartment, a cell selection compartment, a clonal cell expansion compartment, a growth medium compartment, a waste compartment, or any combination thereof. In some instances, the disclosed devices or cartridges may further comprise a cell removal port for removing cells that have been selectively detached from one or more clonal colonies so that they may be subjected to genetic testing or other testing techniques; optically-transparent walls or windows for compatibility with optical imaging and growth monitoring techniques, laser photodetachment techniques, and/or laser photoablation techniques; integrated electrodes for use in performing electroporation; integrated electrodes for performing electrical impedance measurements to monitor cell growth; or any combination thereof.

The functional components of the disclosed apparatus or systems that comprise one or more of the disclosed cell transfection, selection, and clonal expansion cartridges may include a fluidics controller, a temperature controller or incubator, a gas controller, a pneumatics controller, a centrifugation controller, an electronics controller, an optical imaging unit, a laser photodetachment and/or photoablation unit, microplate-handling robotics, a processor, a system controller, or any combination thereof.

The distinctive features of the disclosed devices and systems may give rise to a number of performance advantages including, but not limited to, a significant reduction in the total number of cells required as input for generation of clonal cell populations, a significant reduction in the total number of cells required to be processed for generation of clonal cell populations, a significant reduction in cell culture reagent and consumables consumption, improvements in cell transfection efficiency, improvements in the overall efficiency of generating clonal cell populations, and higher overall throughput (i.e., reduced start to finish times) for generating clonal cell populations.

As noted, this disclosure provides methods, devices, and systems for performing cell transfection, selection, and expansion of clonal cell populations. Various aspects of the disclosed methods, devices, and systems described herein may be applied to any of the particular applications set forth below, or for any other types of cell line engineering application. It shall be understood that different aspects of the disclosed methods, devices, and systems can be appreciated individually, collectively, or in combination with each other.

### Definitions

Unless otherwise defined, the technical terms used herein have the same meaning as commonly understood by one of ordinary skill in the art in the field to which this disclosure belongs.

As used in this specification and the appended claims, the singular forms “a”, “an”, and “the” include plural references unless the context clearly dictates otherwise. Any reference to “or” herein is intended to encompass “and/or” unless otherwise stated.

As used herein, the term ‘about’ a number refers to that number plus or minus 10% of that number. The term ‘about’ when used in the context of a range refers to that range minus 10% of its lowest value and plus 10% of its greatest value.

As used herein, the term “transfection” is used broadly to indicate not only the process of introducing nucleic acids into eukaryotic cells, but also, e.g., bacterial transformation (in which bacteria take up foreign genetic material (e.g., DNA) from the environment) and bacterial transduction (in which genes from a host bacterium are incorporated into the genome of a bacterial virus (bacteriophage) and then carried to another host bacterium when the bacteriophage infects the new host. Thus, as used herein, the term “transfection” is used broadly to indicate any process for introducing a nucleic acid into any type of cell (notwithstanding its use as a term of art). In some instances, e.g., in the case of a CRISPR edit, the “transfection” process may include introduction of one or more nucleic acid molecule(s) that code for one or more Cas protein(s) and/or one or more guide RNA(s) or may include introduction of the one or more Cas proteins themselves and/or one or more guide RNA(s) (e.g., where the guide RNA(s) may be pre-bound to the Cas protein or not pre-bound to the Cas protein(s)). In some instances, e.g., in the case of a CRISPR edit, the “transfection” process may include introduction of multiple guide RNAs, multiple enzymes, multiple DNA repair templates, etc.

As used herein, the terms “device”, “microfluidic device”, and “cartridge” are used interchangeably when referring to the disclosed devices for performing one or more of: cell transfection, cell selection and/or clonal cell expansion.

As used herein, the term “fluid” may refer to a gas, a liquid, or in some instances, to a gel (e.g., a hydrogel) that is sufficiently soft and deformable as to have fluid-like properties.

As used herein, the terms “fluid channel” or simply “channel” are used interchangeably when referring to the disclosed devices for performing cell transfection, cell selection, clonal cell expansion, or any combination thereof. Similarly, a “fluid inlet” may be referred to simply as an “inlet, and a “fluid outlet” may be referred to simply as an “outlet”.

As used herein, the terms “fluid compartment”, “fluid chamber”, and “fluid reservoir”, or simply “compartment”, “chamber”, or “reservoir”, are used interchangeably when referring to the disclosed devices for performing cell transfection, cell selection, clonal cell expansion, or any combination thereof. In some instances, a “compartment”, “chamber”, or “reservoir” may comprise a specific region on a substrate surface. In some instances, a “compartment”, “chamber”, “reservoir”, or “region” may be enclosed and sealed by a lid. In some instances, a “compartment”, “chamber”, “reservoir”, or “region” may be enclosed by a removable lid so that the interior of the “compartment”, “chamber”, “reservoir”, or “region” is accessible.

As used herein, the terms “controller”, “module”, and “unit” are used interchangeably when referring to components or sub-systems of the disclosed apparatus or systems for performing cell transfection, cell selection, clonal cell expansion, or any combination thereof.

As used herein, the term laser photodetachment is used in a general sense to include various related mechanisms by which cells may be disrupted or destroyed upon exposure to light, e.g., intense light, at various wavelengths (ranging from ultraviolet (UV) wavelengths to infrared (IR) wavelengths) in either a pulsed or continuous wave mode.

As used herein, the terms “laser photoablation”, “photoablation”, and simply “ablation” are used interchangeably and in a general sense to include various related mechanisms by which cells may be disrupted or destroyed upon exposure to light, e.g., intense light, at various wavelengths (ranging from ultraviolet (UV) wavelengths to infrared (IR) wavelengths) in either a pulsed or continuous wave mode.

Cells: The disclosed methods and systems may be used for preparation of clonal populations of any of a variety of cells known to those of skill in the art. In some aspects, the cells may be any adherent and non-adherent eukaryotic cell, mammalian cell, primary or immortalized human cell or cell line, primary or immortalized rodent cell or cell line, cancer cells, normal or diseased human cells derived from any of a variety of different organs or tissue types (e.g., white blood cells, red blood cells, epithelial cells, endothelial cells, neurons, glial cells, astrocytes, fibroblasts, skeletal muscle cells, smooth muscle cells, gametes, or cells from the heart, lungs, brain, liver, kidney, spleen, pancreas, thymus, bladder, stomach, colon, small intestine), distinct cell subsets such as immune cells, CD8+ T cells, CD4+ T cells, CD44^{high}/CD24^{low} cancer stem cells, Lgr5/6+ stem cells, undifferentiated human stem cells, human stem cells that have been induced to differentiate, rare cells (e.g., circulating tumor cells (CTCs), circulating epithelial cells, circulating endothelial cells, circulating endometrial cells, bone marrow cells, progenitor cells, foam cells, mesenchymal cells, or trophoblasts), animal cells (e.g., mouse, rat, pig, dog, cow, or horse), plant cells, yeast cells, fungal cells, bacterial cells, algae cells, adherent or non-adherent prokaryotic cells, or any combination thereof. In some aspects, the cells may be immune cells, e.g., T cells, cytotoxic (killer) T cells, helper T cells, alpha beta T cells, gamma delta T cells, T cell progenitors, B cells, B-cell progenitors, lymphoid stem cells, myeloid progenitor cells, lymphocytes, granulocytes, Natural Killer cells, plasma cells, memory cells, neutrophils, eosinophils, basophils, mast cells, monocytes, dendritic cells, macrophages, or any combination thereof.

As noted, in some instances the disclosed methods and systems may be used to prepare clonal populations of stem cells, e.g., embryonic stem cells, adult (tissue-specific) stem cells, mesenchymal stem cells, or induced pluripotent stem cells. Embryonic stem cells are obtained from the inner cell mass of a blastocyst (a mainly hollow ball of cells that, in the human, forms three to five days after an egg cell is fertilized by a sperm), and are typically pluripotent, i.e., they can be used to generate any of the body's specialized cell types, but typically cannot generate support structures like the placenta and umbilical cord. Adult stem cells are multipotent, i.e., they can typically generate a few different cell types found in a specific tissue or organ. Mesenchymal stem cells (MSCs; sometimes referred to as “stromal cells”) are isolated from, e.g., bone marrow or the stroma (the connective tissue that surrounds other tissues and organs). MSCs derived from bone marrow or other tissues have been shown to be capable of making bone, cartilage and fat cells, although it is unclear if they are actual stem cells or what other cell types they are capable of generating. Their characteristics appear to depend on what tissue they are isolated from and how they are isolated and grown.

In some instances, the disclosed methods and systems may be used to prepare clonal populations of induced pluripotent stem cells (IPSCs), or any differentiated cell line derived therefrom. Induced pluripotent stem cells are derived from, e.g., skin or blood cells that have been reprogrammed to regress into an embryonic-like pluripotent state, and which may subsequently be triggered to differentiate into any of a variety of specific cell types, e.g., beta islet cells, egg and sperm precursors, liver cells, bone precursor cells, blood cells, neurons, and the like, for use in biomedical research and/or therapeutic applications.

Cell culturing methods: In general, the cell culturing conditions used (growth medium, incubation temperature, humidity, O2 concentration, CO2 concentration, etc.) will vary depending on the type of clonal cell populations being prepared. A suitable growth medium provides the essential nutrients (amino acids, carbohydrates, vitamins, minerals, etc.) required by the specific cell type being cultured, maintains the pH and osmotic pressure required by the specific cell type being cultured, and may further comprise growth factors, hormones, etc. The cells may be anchorage-dependent cells that are typically cultured while attached to a solid or semi-solid substrate (e.g., in adherent or monolayer culture). In some cases, the cells may be non-adherent or suspension cells that are typically grown floating in the culture medium (e.g., suspension culture). In some instances, the disclosed methods, devices, and systems may be used to prepare clonal cultures of non-adherent cells that have been allowed to settle on the bottom surface of a cell selection or growth compartment (or on a growth substrate contained therein). In some instances, the disclosed methods, devices, and systems may be used to prepare clonal cultures of non-adherent cells that have been captured and tethered to the bottom surface of a cell selection or growth compartment (or on a growth substrate contained therein), e.g., using tethered capture antibodies directed to a specific cell surface receptor.

Transfection agents: In some instances, the transfection of cells introduced into the disclosed devices or cartridges may be implemented by transfecting the cells with one or more transfection agents comprising one or more types of DNA molecule, RNA molecule, oligonucleotide, aptamer, non-plasmid nucleic acid molecule, ribonucleoprotein (RNP), plasmid, viral vector, cosmid, artificial chromosome, or any combination thereof.

Cell transfection, selection, and clonal expansion cartridges: The functional components of the disclosed devices or cartridges may comprise: one or more fluid inlets (including inlets for introducing cells into the device), one or more fluid outlets (including outlets for removing cells from the device), a cell transfection compartment, a cell selection compartment, a clonal cell expansion compartment, a growth medium compartment, a waste compartment, and one or more interconnecting fluid channels, or any combination thereof. In some instances, the disclosed devices or cartridges may further comprise one or more miniature or microfabricated valves, one or more miniature or microfabricated pumps, one or more integrated sensors (e.g., temperature sensors, pH sensors, oxygen sensors, CO₂ sensors, and the like). In some instances, the disclosed devices or cartridges may comprise assemblies of two or more components, as will be discussed in more detail below.

In some instances, the disclosed devices or cartridges may comprise a cell removal port positioned at an intermediate location between the fluid outlet from a cell selection compartment and a fluid inlet for a clonal cell expansion compartment. In some instances, such a cell removal port may be used, e.g., for removing cells that have been selectively detached from one or more clonal colonies so that they may be subjected to genetic testing;

In some instances, the disclosed devices or cartridges may further comprise optically-transparent walls or windows for compatibility with optical imaging and growth monitoring techniques, laser photodetachment techniques, and/or laser photoablation techniques; integrated electrodes for use in performing electroporation; integrated electrodes for performing electrical impedance measurements to monitor cell growth; or any combination thereof.

Fluid channels: In general, the dimensions of fluid channels in the disclosed devices and cartridges will be optimized to (i) provide efficient delivery of cells and other reagents to one or more fluid compartments, and (ii) to minimize cell suspension and reagent consumption. In some instances, the width of fluid channels may be between 50 microns and 2 mm. In some instances, the width of fluid channels may be at least 50 microns, at least 100 microns, at least 200 microns, at least 300 microns, at least 400 microns, at least 500 microns, at least 750 microns, at least 1 mm, or at least 2 mm. In some instances, the width of fluid channels may at most 2 mm, at most 1 mm, at most 750 microns, at most 500 microns, at most 400 microns, at most 300 microns, at most 200 microns, at most 100 microns, or at most 50 microns. Any of the lower and upper values described in this paragraph may be combined to form a range included within the present disclosure, for example, in some instances the width of a fluid channel may range from about 100 μm to about 1 mm. Those of skill in the art will recognize that the width of a fluid channel may have any value within this range, e.g., about 1.45 mm. In some instances, the dimensions of a specific fluid channel (width, depth, and/or length) may be tuned depending on its specific function.

In some embodiments, the depth of the fluid channels may range from about 50 microns to about 2 mm. In some instances, the depth of a fluid channel may be at least 50 microns, at least 100 microns, at least 200 microns, at least 300 microns, at least 400 microns, at least 500 microns, at least 750 microns, at least 1 mm, at least 1.25 mm, at least 1.5 mm, at least 1.75 mm, or at least 2 mm. In some instances, the depth of a fluid channel may be at most 2 mm, at most 1.75 mm, at most 1.5 mm, at most 1.25 mm, at most 1 mm, at most 750 microns, at most 500 microns, at most 400 microns, at most 300 microns, at most 200 microns, at most 100 microns, or at most 50 microns. Any of the lower and upper values described in this paragraph may be combined to form a range included within the present disclosure, for example, in some instances the depth of a fluid channel may range from about 100 μm to about 400 μm. Those of skill in the art will recognize that the depth of a fluid channel may have any value within this range, e.g., about 555 μm. In some instances, the dimensions of a specific fluid channel (width, depth, and/or length) may be tuned depending on its specific function.

Fluid compartments: In some instances, the disclosed devices or cartridges may comprise at least one, at least two, at least three, at least four, at least five, or more than five fluid compartments. In some instances, each compartment may serve a different function in the sequence of cell processing steps required to generate clonal populations of cells comprising a desired genotype. In some instances, a single fluid compartment may serve two or more different functions in the sequence of cell processing steps required to generate clonal populations of cells.

In some instances, each fluid compartment may have at least one, at least two, at least three, at least four, or at least five fluid inlets (or cell introduction ports). In some instances, each fluid compartment may have at least one, at least two, at least three, at least four, or at least five fluid outlets (or cell removal ports).

In some instances, any given dimension (e.g., the shortest dimension or the longest dimension, or the length, width, or height/depth) of a fluid compartment of the disclosed devices and cartridges may range from about 0.1 mm to about 20 cm. In some instances, a given dimension of a fluid compartment may be at least 0.1 mm, at least 0.2 mm, at least 0.3 mm, at least 0.4 mm, at least 0.5 mm, at least 0.6 mm, at least 0.7 mm, at least 0.8 mm, at least 0.9 mm, at least 1 mm, at least 2 mm, at least 3 mm, at least 4 mm, at least 5 mm, at least 1 cm, at least 1.5 cm, at least 2 cm, at least 2.5 cm, at least 3 cm, at least 3.5 cm, at least 4 cm, at least 4.5 cm, at least 5 cm, at least 5.5 cm, at least 6 cm, at least 6.5 cm, at least 7 cm, at least 7.5 cm, at least 8 cm, at least 8.5 cm, at least 9 cm, at least 9.5 cm, at least 10 cm, at least 12 cm, at least 14 cm, at least 16 cm, at least 18 cm, or at least 20 cm. In some instances, a given dimension of a fluid compartment may be at most 20 cm, at most 18 cm, at most 16 cm, at most 14 cm, at most 12 cm, at most 10 cm, at most 9.5 cm, at most 9 cm, at most 8.5 cm, at most 8 cm, at most 7.5 cm, at most 7 cm, at most 6.5 cm, at most 6 cm, at most 5.5 cm, at most 5 cm, at most 4.5 cm, at most 4 cm, at most 3.5 cm, at most 3 cm, at most 2.5 cm, at most 2 cm, at most 1.5 cm, at most 1 cm, at most 5 mm, at most 4 mm, at most 3 mm, at most 2 mm, at most 1 mm, at most 0.9 mm, at most 0.8 mm, at most 0.7 mm, at most 0.6 mm, at most 0.5 mm, at most 0.4 mm, at most 0.3 mm, at most 0.2 mm, or at most 0.1 mm. Any of the lower and upper values described in this paragraph may be combined to form a range included within the present disclosure, for example, in some instances a given dimension of a fluid compartment may range from about 1 mm to about 5 cm. Those of skill in the art will recognize that a given dimension of a fluid compartment may have any value within this range, e.g., about 5.45 cm. In some instances, the dimensions of a specific fluid compartment may be tuned depending on its specific function.

In some instances, the volume of any given fluid compartment of the disclosed devices and cartridges may range from about 1 μl to about 100 ml. In some instances, the volume of any given fluid compartment may be at least 1 μl, at least 5 μl, at least 10 μl, at least 50 μl, at least 100 μl, at least 200 μl, at least 300 μl, at least 400 μl, at leat 500 μl, at least 600 μl, at least 700 μl, at least 800 μl, at least 900 μl, at least 1 nl, at least 5 nl, at least 10 nl, at least 50 nl, at least 100 nl, at least 200 nl, at least 300 nl, at least 400 nl, at least 500 nl, at least 600 nl, at least 700 nl, at least 800 nl, at least 900 nl, at least 1 at least 5 at least 10μ, at least 50 at least 100 at least 200 at least 300 at least 400 at least 500 at least 600 at least 700 at least 800 at least 900 at least 1 ml, at least 2 ml, at least 3 ml, at least 4 ml, at least 5 ml, at least 6 ml, at least 7 ml, at least 8 ml, at least 9 ml, at least 10 ml, at least 20 ml, at least 30 ml, at least 40 ml, at least 50 ml, at least 60 ml, at least 70 ml, at least 80 ml, at least 90 ml, or at least 100 ml. In some instances, the volume of any given fluid compartment may be at most 100 ml, at most 90 ml, at most 80 ml, at most 70 ml, at most 60 ml, at most 50 ml, at most 40 ml, at most 30 ml, at most 20 ml, at most 10 ml, at most 9 ml, at most 8 ml, at most 7 ml, at most 6 ml, at most 5 ml, at most 4 ml, at most 3 ml, at most 2 ml, at most 1 ml, at most 900 at most 800 at most 700 pl, at most 600 pl at most 500 pl, at most 400 pl, at most 300 pl, at most 200 pl, at most 100 at most 50 at most 10 at most 5 at most 1 at most 900 nl, at most 800 nl, at most 700 nl, at most 600 nl, at most 500 nl, at most 400 nl, at most 300 nl, at most 200 nl, at most 100 nl, at most 50 nl, at most 10 nl, at most 5 nl, at most 1 nl, at most 900 pl, at most 800 pl, at most 700 pl, at most 600 pl, at most 500 pl, at most 400 pl, at most 300 pl, at most 200 pl, at most 100 pl, at most 50 pl, at most 10 pl, at most 5 pl, or at most 1 pl. Any of the lower and upper values described in this paragraph may be combined to form a range included within the present disclosure, for example, in some instances the volume of any given fluid compartment may range from about 800 μl to about 20 ml. Those of skill in the art will recognize that the volume of any given fluid compartment may have any value within this range, e.g., about 4.8 ml. In some instances, the volume of a specific fluid compartment may be tuned depending on its specific function.

Cell transfection compartments: In some instances, one or more fluid compartments may function as cell transfection compartments configured to perform any of a variety of transfection methods known to those of skill in the art. Examples include, but are not limited to, chemical transfection, mechanical transfection, electroporation, photoporation, and the like. In some cases, e.g., if the cell transfection compartment is configured to perform electroporation, the fluid compartment may comprise additional structural features, e.g., a constricted fluid flow path, or one or more pairs of electrodes, or optically-transparent walls.

Cell selection compartments: In some instances, one or more fluid compartments may function as cell selection compartments in which single cells (e.g., adherent cells or suspension cells) are allowed to attach to (or are tethered to) a surface and initiate the formation of a clonal cell cluster. In some instances, the cell selection compartments may be configured to include a surface coating layer designed to facilitate cell attachment, or to facilitate laser-based photodetachment or photoablation. In some instances, the cell selection compartments may be configured with at least one optically-transparent wall so that cell growth may be monitored, e.g., by means of imaging. In some instances, the cell selection compartments may be configured with at least one pair of electrodes so that cell growth may be monitored, e.g., by means of electrical impedance measurements. In some instances, the cell selection compartments may comprise both an optically-transparent wall and at least one pair of electrodes.

As noted, in some instances the cell selection compartment (or one or more walls or surfaces therein) may be configured to include a surface coating layer designed to facilitate cell attachment, or to facilitate laser-based photodetachment or photoablation. Examples of suitable surface coatings include, but are not limited to, an α-poly-lysine coating, a collagen coating, a poly-1-ornithine, a fibronectin coating, and a laminin coating. In some instances, e.g., where the disclosed devices or cartridges are used with induced pluripotent stem cells (iPSCs) derived directly from adult cells, the surface coating may comprise the Synthemax™ vitronectin coating (Corning, Inc., Corning N.Y.) and/or iMatrix-511 recombinant laminin coating (Takara Bio USA, Mountain View, Calif.). In some instances, the cell selection compartment (or one or more walls or surfaces therein) may be treated with a surface treatment to facilitate cell attachment or a surface coating layer. Examples include, but are not limited to, a plasma treatment.

In some instances, the cell selection compartment may not have any internal structure such as sub-compartments, single cell traps, or single cell chambers. In some instances, the cell selection compartment may comprise two or more sub-compartments, single cell traps, or single cell chambers within which individual cells may be compartmentalized. In some instances, cells may be introduced to the sub-compartments, single cell traps, or single cell chambers using, e.g., hydrodynamic forces to trap individual cells, magnetic beads to which individual cells are tethered and externally-applied magnetic fields, or optical tweezers to move and position individual cells. In some instances, cell transfection may be performed within the sub-compartments, single cell traps, or single cell chambers within the cell selection compartment, e.g., using a laser-based poration technique to transiently disrupt the cell membrane and allow a transfection agent dissolved in the cell culture medium or buffer within which the cells reside to enter the cells.

Any of a variety of cell selection techniques known to those of skill in the art may be performed within a cell selection compartment, as will be discussed in more detail below. In some instances, a cell selection compartment may also function as a clonal cell expansion compartment.

Cell expansion compartments: In some instances, one or more fluid compartments may function as clonal cell expansion compartments in which a single cell or small clonal cell cluster is subjected to one or more cycles of cell growth and expansion. In some instances, the cell selection compartments may be configured to include a surface coating layer designed to facilitate cell attachment. In some instances, the cell expansion compartments may be configured with at least one optically-transparent wall so that cell growth may be monitored, e.g., by means of imaging. In some instances, the cell expansion compartments may be configured with at least one pair of electrodes so that cell growth may be monitored, e.g., by means of electrical impedance measurements. In some instances, the cell expansion compartments may comprise both an optically-transparent wall and at least one pair of electrodes.

In some instances, cell expansion compartments (or one or more walls or surfaces therein) may also comprise a surface coating layer and/or surface treatment designed to facilitate cell attachment or to facilitate cell detachment, e.g., using chemical means, enzymatic means (e.g., trypsin treatment), laser-based photodetachment, and the like. Examples of suitable surface coatings include, but are not limited to, an α-poly-lysine coating, a collagen coating, a poly-1-ornithine, a fibronectin coating, and a laminin coating. In some instances, e.g., where the disclosed devices or cartridges are used with induced pluripotent stem cells (iPSCs) derived directly from adult cells, the surface coating may comprise the Synthemax™ vitronectin coating (Corning, Inc., Corning N.Y.) and/or iMatrix-511 recombinant laminin coating (Takara Bio USA, Mountain View, Calif.). In some instances, the cell selection compartment (or one or more walls or surfaces therein) may be treated with a surface treatment to facilitate cell attachment or a surface coating layer. Examples include, but are not limited to, a plasma treatment.

In some embodiments, the cell selection compartment comprises a pattern of indentations on an inner surface and/or a pattern of a substrate on an inner surface. In certain embodiments, the cell expansion compartment comprises a pattern of indentations on an inner surface and/or a pattern of a substrate on an inner surface. In some embodiments, the substrate is a protein substrate. In certain embodiments, the pattern of indentations and/or the pattern of a substrate are configured to prevent cell migration within the compartments. A pattern can be established in such chambers consisting of regions where cells attach easily and regions where cells attach poorly. Regions where cells attach could consist of micro printed/stamped adhesive substrates including but not limited to poly-D-Lysine (MW), poly-L-Ornithine, Lamnin, fibronectin and vitronectin. Non-limiting example of poor cell adhesion surface include hydrophobic surfaces such as silane coatings and untreated labware plastic, which can be used to generate regions of poor cell attachment by constructing such regions out of untreated plastics such as polystyrene, COC, COP, silane etc.

Growth medium reservoirs: In some instances, one or more fluid compartments may function as growth medium reservoirs which function as sources of fresh growth medium for maintaining and expanding cell cultures within the device. In some instances, growth medium reservoirs within the disclosed devices or cartridges may comprise a gas permeable membrane which allows any trapped air or other gas to be removed from the compartment, and which additionally may be utilized in applying pressure to the growth medium contained therein to drive a flow of the growth medium fluid through the fluid channels and fluid compartments of the device.

Waste reservoirs: In some instances, one or more fluid compartments within the disclosed devices or cartridges may function as waste reservoirs which function as storage compartments for containing spent growth medium or other fluids removed from the cell transfection, cell selection, and/or cell culture expansion compartments of the device.

Device and cartridge fabrication and assembly: In some instances, the disclosed devices and cartridges may be fabricated as monolithic structures from a single material. In some instances, the disclosed devices or cartridges may comprise assemblies of two or more components that are bonded or otherwise fastened together to create the completed device.

Cartridges may be fabricated using a variety of techniques and materials known to those of skill in the art. In general, the cartridges may be fabricated as a series of separate component parts and subsequently assembled using any of a variety of mechanical assembly or bonding techniques. Examples of suitable fabrication techniques include, but are not limited to, photolithography and chemical etching, conventional machining, CNC machining, injection molding, thermoforming, and 3D printing. Once the cartridge components have been fabricated they may be mechanically assembled using screws, clips, and the like, or permanently bonded using any of a variety of techniques (depending on the choice of materials used), for example, through the use of thermal or ultrasonic bonding/welding or any of a variety of adhesives or adhesive films, including epoxy-based, acrylic-based, silicone-based, UV curable, polyurethane-based, or cyanoacrylate-based adhesives.

Cartridge components may be fabricated using any of a number of suitable materials, including but not limited to silicon, fused-silica, glass, any of a variety of polymers, e.g., polydimethylsiloxane (PDMS; elastomer), polymethylmethacrylate (PMMA), polycarbonate (PC), polypropylene (PP), polyethylene (PE), high density polyethylene (HDPE), polyimide, cyclic olefin polymers (COP), cyclic olefin copolymers (COC), polyethylene terephthalate (PET), epoxy resins, or metals (e.g., aluminum, stainless steel, copper, nickel, chromium, and titanium), flexdym.

As noted, in some instances, the disclosed devices or cartridges (or one or more walls or surfaces of one or more fluid channels and/or fluid compartments contained therein) may be fabricated using an optically-transparent material. Such optically-transparent windows, walls, or surfaces may be designed to facilitate optical imaging of cells within the device (e.g., using fluorescence imaging or other imaging techniques), or to facilitate optical monitoring of cells or cell processing steps within the device (e.g., using a spectroscopic measurement technique). In some instances, such optically-transparent windows, walls, or surfaces may be designed to facilitate the use of laser-based photodetachment and/or photoablation of cells within the device. In some instances, the optically-transparent windows, walls or surfaces of the disclosed devices and cartridges may be transparent in the ultraviolet (UV), visible, or near-infrared (near-IR) regions of the electromagnetic spectrum, or any combination thereof. In some instances, the optically-transparent windows, walls, or surfaces may be transparent in a wavelength range centered at about 355 nm. In some instances, the optically-transparent windows, walls, or surfaces may be transparent in a wavelength range centered at about 785 nm. In some instances, the optically-transparent windows, walls, or surfaces may be transparent in the wavelength range from about 1440 nm to about 1450 nm.

In some instances, the optically-transparent windows, walls, or surfaces of the disclosed devices or cartridges may provide for transmission of light at a wavelength ranging from about 220 nm (UV light) to about 1500 nm (IR light). In some instances, the wavelength of the transmitted light may be at least 220 nm, at least 250 nm, at least 300 nm, at least 350 nm, at least 400 nm, at least 450 nm, at least 500 nm, at least 550 nm, at least 600 nm, at least 650 nm, at least 700 nm, at least 750 nm, at least 800 nm, at least 850 nm, at least 900 nm, at least 950 nm, at least 1,000 nm, at least 1,100 nm, at least 1,200 nm, at least 1,300 nm, at least 1,400 nm, or at least 1,500 nm. In some instances, the wavelength of the transmitted light may be at most 1,500 nm, at most 1,400 nm, at most 1,300 nm, at most 1,200 nm, at most 1,100 nm, at most 1,000 nm, at most 950 nm, at most 900 nm, at most 850 nm, at most 800 nm, at most 750 nm, at most 700 nm, at most 650 nm, at most 600 n, at most 550 nm, at most 500 nm, at most 450 nm, at most 400 nm, at most 350 nm, at most 300 nm, at most 250 nm, or at most 220 nm. Any of the lower and upper values described in this paragraph may be combined to form a range included within the present disclosure, for example, in some instances the wavelength of the transmitted light may range from about 400 nm to about 900 nm. Those of skill in the art will recognize that the wavelength of the transmitted light may have any value within this range, e.g., about 855 nm.

The fluid inlets and/or fluid outlets of the cartridge may be designed to provide convenient and leak-proof fluid connections with an external instrument or may serve as open reservoirs for manual pipetting of cell samples and reagents into or out of the cartridge. Examples of convenient mechanical designs for the inlet and outlet port connectors include, but are not limited to, threaded connectors, swaged connectors, Luer lock connectors, Luer slip or “slip tip” connectors, press fit connectors, and the like. In some instances, the fluid inlets and/or fluid outlets of the cartridge may further comprise caps, spring-loaded covers or closures, phase change materials, or polymer membranes that may be opened or punctured when the cartridge is positioned in an instrument, and which serve to prevent contamination of internal cartridge surfaces during storage and/or which prevent fluids from spilling when the cartridge is removed from an instrument.

In some instances, the overall dimensions (or “footprint”) of the disclosed devices or cartridges may range from about 10 mm to about 150 mm in either length and/or width. In some instances, the disclosed devices or cartridges may be at least 10 mm, at least 15 mm, at least 20 mm, at least 25 mm, at least 30 mm, at least 35 mm, at least 40 mm, at least 45 mm, at least 50 mm, at least 55 mm, at least 60 mm, at least 65 mm, at least 70 mm, at least 75 mm, at least 80 mm, at least 85 mm, at least 90 mm, at least 95 mm, at least 100 mm, at least 105 mm, at least 105 mm, at least 110 mm, at least 115 mm, at least 120 mm, at least 125 mm, at least 130 mm, at least 135 mm, at least 140 mm, at least 145 mm, or at least 150 mm in either length and/or width. In some instances, the disclosed devices or cartridges may be at most 150 mm, at most 145 mm, at most 140 mm, at most 135 mm, at most 130 mm, at most 125 mm, at most 120 mm, at most 115 mm, at most 110 mm, at most 105 mm, at most 100 mm, at most 95 mm, at most 90 mm, at most 85 mm, at most 80 mm, at most 75 mm, at most 70 mm, at most 65 mm, at most 60 mm, at most 55 mm, at most 50 mm, at most 45 mm, at most 40 mm, at most 35 mm, at most 30 mm, at most 25 mm, at most 20 mm, at most 15 mm, or at most 10 mm in either length and/or width. Any of the lower and upper values described in this paragraph may be combined to form a range included within the present disclosure, for example, in some instances the length and/or width of the disclosed devices or cartridges may range from about 30 mm to about 130 mm. Those of skill in the art will recognize that the length and/or width of the disclosed devices or cartridges may have any value within this range, e.g., about 112.5 mm.

In some instances, the dimensions of the disclosed devices or cartridges may have a tolerance (in length, width, and/or depth) that ranges from about ±0.005 mm to about ±0.05 mm. In some instances, the tolerance in any given dimension may be within ±0.005 mm, 0.006 mm, ±0.007 mm, ±0.008 mm, ±0.009 mm, ±0.01 mm, ±0.02 mm, ±0.03 mm, ±0.04 mm, or ±0.05 mm. Those of skill in the art will recognize that the tolerance in any given dimension may have any value within this range, e.g., about ±0.0055 mm.

In a preferred instance, the dimensions or “footprint” of the disclosed devices or cartridges may comply with American National Standards Institute (ANSI) Standard Number SLAS 4-2004 (R2012) so that they are compatible with existing laboratory automation equipment, e.g., microplate-handling robotics, available from other manufacturers. In these instances, the device or cartridge may have a footprint that is 127.76 mm±0.5 mm in length and 85.48 mm±0.5 mm in width.

Other device or cartridge components: As indicated above, in some instances the disclosed devices or cartridges may include integrated miniature or microfabricated pumps or other fluid actuation mechanisms for control of fluid flow through the device. Examples of suitable miniature pumps or fluid actuation mechanisms include, but are not limited to, electromechanically- or pneumatically-actuated miniature syringe or plunger mechanisms, chemical propellants, membrane diaphragm pumps actuated pneumatically or by an external piston, pneumatically-actuated reagent pouches or bladders, or electro-osmotic pumps.

As noted above, in some instances the disclosed devices or cartridges may include miniature or microfabricated valves for compartmentalizing pre-loaded reagents and/or controlling fluid flow through the device. Examples of suitable miniature valves include, but are not limited to, one-shot “valves” fabricated using wax or polymer plugs that can be melted or dissolved, or polymer membranes that can be punctured; pinch valves constructed using a deformable membrane and pneumatic, hydraulic, magnetic, electromagnetic, or electromechanical (solenoid) actuation, one-way valves constructed using deformable membrane flaps, rotary valves and miniature gate valves.

In some instances, the disclosed devices or cartridges may include vents for providing an escape path for trapped air or other gases. Vents may be constructed according to a variety of techniques known to those of skill in the art, for example, using a porous plug of polydimethylsiloxane (PDMS) or other hydrophobic material that allows for capillary wicking of air but blocks penetration by water. Vents may also be constructed as apertures through hydrophobic barrier materials, such that wetting to the aperture walls does not occur at the pressures used during operation.

In general, the mechanical interface features of the disclosed devices or cartridge provide for easily removable but highly precise and repeatable positioning of the cartridge relative to an external instrument system. Suitable mechanical interface features include, but are not limited to, alignment pins, alignment guides, mechanical stops, and the like.

In some instances, the disclosed devices or cartridges may also include temperature control components or thermal interface features for mating to external temperature control modules. Examples of suitable temperature control elements include, but are not limited to, resistive heating elements, miniature infrared-emitting light sources, Peltier heating or cooling devices, heat sinks, thermistors, thermocouples, and the like. Thermal interface features will typically be fabricated from materials that are good thermal conductors (e.g., copper, gold, silver, aluminum, etc.) and may comprise one or more flat surfaces capable of making good thermal contact with external heating blocks or cooling blocks.

In some instances, the disclosed devices or cartridges, or one or more of the individual fluid compartments contained therein, may further comprise one or more sensors for use in monitoring and regulating the microenvironment of cells grown within the device to optimize and maintain cell viability. Examples include, but are not limited to, temperature sensors, pH sensors, gas sensors (e.g., O₂ sensors, CO₂ sensors), glucose sensors, optical sensors, electrochemical sensors, optoelectronic sensors, piezoelectric sensors, or any combination thereof.

As noted above, in some instances the disclosed devices or cartridges (or fluid channels and/or fluid compartments contained therein) may further comprise additional components or features, e.g., transparent optical windows, micro-lens components, or light-guiding features to facilitate microscopic observation or spectroscopic monitoring techniques, inlet and outlet ports for making connections to perfusion systems, electrical connections for connecting electrodes or sensors to external processors or power supplies, etc.

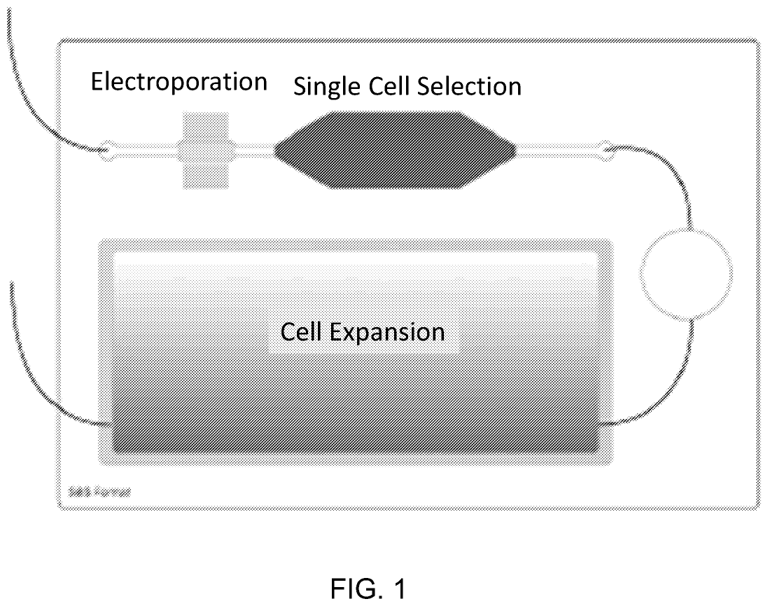

FIG. 1 provides a non-limiting example of the layout of a cell transfection, selection, and clonal expansion cartridge of the present disclosure. The device comprises an inlet (upper left) that is in fluid communication with a cell transfection compartment (e.g., an electroporation chamber comprising a pair of electrodes), the fluid outlet of which is in fluid communication with an inlet to a cell selection compartment. The outlet of the cell selection compartment is in fluid communication with a valve (indicated as a circular feature) which provides means for removing cells that have been detached from a clonal cell colony growing on a surface within the cell selection compartment so that they may be subjected to genetic testing for identifying those cell colonies that present the desired genotype. The valve is also in fluid communication with the inlet of a cell expansion compartment so that the remaining cells from a selected clonal cell colony may be detached and transferred to the cell expansion compartment based on the genetic testing results. Following several cycles of cell growth and division, the clonal population of cells may be harvested from the cell expansion compartment, e.g., by trypsin treatment to detach them from a growth surface within the cell expansion compartment and removing them from the outlet depicted at the lower left of the device in FIG. 1.

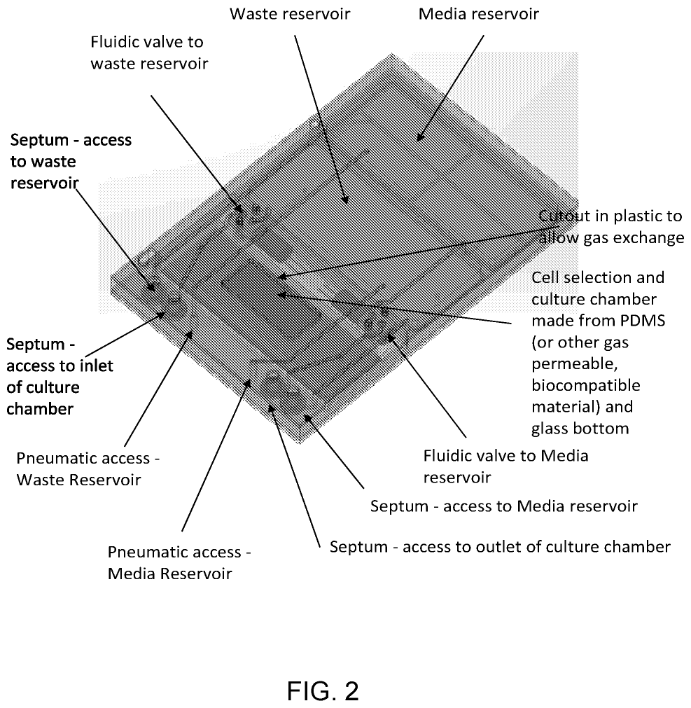

FIG. 2 provides a non-limiting illustration of the layout of fluid inlets, fluid outlets, fluid channels and fluid compartments (e.g., for cell transfection, selection, and growth, as well as for storage of culture medium and waste) in one instance of the disclosed cartridges. In this non-limiting example, culture medium is supplied to a fluid compartment (cell culture chamber) used for performing cell selection and clonal cell growth from a media reservoir. The device also includes a waste reservoir as well as inlets and outlets for accessing the culture medium reservoir, waste reservoir, and cell culture chamber. In some instances, the fluid inlets and outlets may comprise septa as indicated in the figure to maintain sterile culture conditions and prevent leakage. In some instances, the device may comprise pneumatic access ports for use in pneumatic control of fluid flow into and out of one or more fluid compartments, e.g., culture medium reservoirs or waste reservoirs, where the fluid compartment may comprise a flexible membrane used to exert pressure on the fluid contained within the compartment. In some instances, the device may be used with a centrifugation device to control fluid flow into and out of one or more fluid compartments, e.g., culture medium reservoirs or waste reservoirs. In some instances, the device may comprise one or more fluidic valves used to control the flow of fluid into, out of, or between fluid compartments, or to introduce or remove cells from the device.

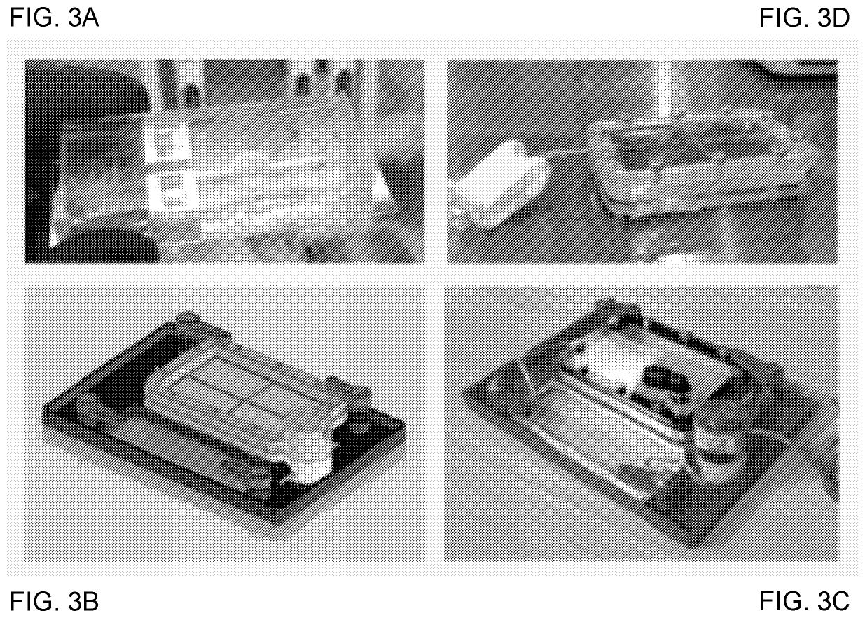

FIGS. 3A-3D provide views of a partially-assembled and fully-assembled cell transfection, selection, and clonal expansion cartridge of the present disclosure. FIG. 3A provides a photograph of a molded PDMS chip with round culture area and electroporation chamber shown with embedded aluminum electrodes. FIG. 3B provides an illustration of the cartridge comprising the base plate and attached cell selection chamber, cell expansion chamber, growth medium reservoir, waste reservoir, and a valve that is in fluid communication with the outlet of the cell selection chamber and the inlet of the cell expansion chamber which provides for removal of cells from the selection compartment for testing. FIG. 3C provides a photograph of a prototype cartridge comprising the base plate and attached cell selection chamber, cell expansion chamber, growth medium reservoir, waste reservoir, and a valve that is in fluid communication with the outlet of the cell selection chamber and the inlet of the cell expansion chamber which provides for removal of cells from the selection compartment for genetic testing. FIG. 3D provides a photograph of the assembled cartridge that includes media and waste reservoirs.

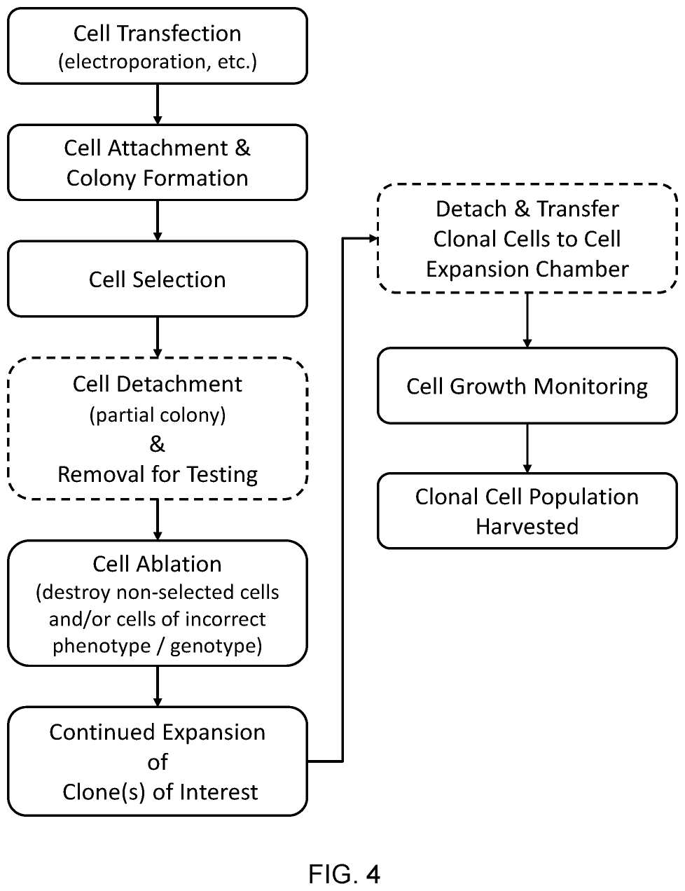

Methods of use: FIG. 4 provides a non-limiting example of the process steps performed within a cell transfection, selection, and clonal expansion cartridge of the present disclosure. The disclosed devices and cartridges allow one to generate clonal populations of genetically-modified cells with high efficiency using a small number of input cells while minimizing reagent consumption and space requirements. The process steps performed within the disclosed devices or cartridges may include: (i) cell transfection (e.g., using electroporation or any of a number of other transfection mechanisms known to those of skill in the art), (ii) cell attachment and colony formation (in some instances, cells may be grown in suspension or in a gel-like matrix rather than on a surface), (iii) cell selection, (iv) partial colony detachment and cell removal for testing (an optional step depending on the configuration of the cartridge and system), (v) cell ablation, (vi) clonal expansion of one or more selected cell colonies, (vii) detachment and transfer of the selected cell colonies to a separate cell expansion chamber (an optional step depending on the configuration of the cartridge and system), (viii) cell growth monitoring, and (ix) cell detachment and harvesting of clonal cell populations, or any combination thereof.

Cell transfection: In some instances, the disclosed devices and cartridges comprise at least one cell transfection compartment that may be configured to perform any of a variety of cell transfection techniques known to those of skill in the art. Examples include, but are not limited to, chemical transfection, mechanical transfection (squeezing), electroporation, laser-induced photoporation, needle-based poration, impalefection, magnetofection, sonoporation, or any combination thereof, where the configuration of the cell transfection compartment is designed to meet the requirements of the specific transfection technique (or combination of techniques) selected. In some instances, the cell transfection compartment may be a fluid channel of the same or different dimensions that those of other fluid channels within the device. In some instances, cell transfection may be performed in the same compartment as one or more of the cell selection, cell detachment and/or ablation, or cell expansion process steps.

Chemical transfection: In some instances, chemical transfection may be implemented, for example, simply by providing a separate fluid channel for introducing and mixing a chemical transfection reagent (e.g., calcium phosphate, dendrimers, cationic polymers such as diethylethanolamine (DEAE)-dextran or polyethylenimine (PEI), and the like that transiently disrupt cell membranes or that bind nucleic acids and facilitate transport across cell membranes) with a cell suspension introduced through a cell inlet channel.

Mechanical transfection: In some instances, mechanical transfection (squeezing) may be implemented, for example, by providing a constriction in a fluid channel through which cells are forced to flow at an appropriate velocity such that the cell membrane is transiently disrupted, thereby allowing a transfection agent suspended in the same medium as the cells to pass through the cell membrane. In some instances, the constriction used to mechanically disrupt cell membranes may range from about 1 micrometer (μm) to about 10 micrometers in width, height, or diameter. In some instances, the constriction may be at least 1 μm, at least 2 μm, at least 3 μm, at least 4 μm, at least 5 μm, at least 6 μm, at least 7 μm, at least 8 μm, at least 9 μm, or at least 10 μm in width, height, or diameter. In some instances, the constriction may be at most 10 μm, at most 9 μm, at most 8 μm, at most 7 μm, at most 6 μm, at most 5 μm, at most 4 μm, at most 3 μm, at most 2 μm, or at most 1 μm in width, height, or diameter. Any of the lower and upper values described in this paragraph may be combined to form a range included within the present disclosure, for example, in some instances the constriction may range from about 2 μm to about 8 μm in width, height, or diameter. Those of skill in the art will recognize that the constriction may have any value within this range, e.g., about 7.6 μm in width, height, or diameter.

In some instances, the constriction may have a length or dimension in the direction of fluid flow and cell transport through the constriction ranging from about 1 μm to about 50 μm. In some instances, the constriction length may be at least 1 μm, at least 2 μm, at least 3 μm, at least 4 μm, at least 5 μm, at least 6 μm, at least 7 μm, at least 8 μm, at least 9 μm, at least 10 μm, at least 15 μm, at least 20 μm, at least 30 μm, at least 40 μm, or at least 50 μm. In some instances, the constriction length may be at most 50 μm, at most 40 μm, at most 30 μm, at most 20 μm, at most 15 μm, at most 10 μm, at most 9 μm, at most 8 μm, at most 7 μm, at most 6 μm, at most 5 μm, at most 4 μm, at most 3 μm, at most 2 μm, or at most 1 μm. Any of the lower and upper values described in this paragraph may be combined to form a range included within the present disclosure, for example, in some instances the constriction length may range from about 5 μm to about 15 μm. Those of skill in the art will recognize that the constriction length may have any value within this range, e.g., about 22.5 μm.

In some instances, a fluid channel or fluid compartment comprising one or more constrictions used to perform mechanical transfection may comprise 1, 2, 3, 4, 5, 6, 7, 8, 9, 10, 20, 30, 40, 50, 60, 70, 80, 90, 100, 200, 300, 400, 500, 600, 700, 800, 900, 1,000, or more than constrictions in series and/or in parallel. In some instances, a fluid channel or fluid compartment comprising one or more constrictions used to perform mechanical transfection may include any number of constrictions (in series and/or in parallel) within this range, e.g., 22 constrictions.

In some instances, cells may be passed through a constriction used to perform mechanical transfection at a flow rate or velocity ranging from about 1 mm/sec to about 1,400 mm/sec. In some instances, the velocity may be at least 1 mm/sec, at least 10 mm/sec, at least 20 mm/sec, at least 30 mm/sec, at least 40 mm/sec, at least 50 mm/sec, at least 60 mm/sec, at least 70 mm/sec, at least 80 mm/sec, at least 90 mm/sec, at least 100 mm/sec, at least 200 mm/sec, at least 300 mm/sec, at least 400 mm/sec, at least 500 mm/sec, at least 600 mm/sec, at least 700 mm/sec, at least 800 mm/sec, at least 900 mm/sec, at least 1,000 mm/sec, at least 1,100 mm/sec at least 1,200 mm/sec, at least 1,300 mm/sec, or at least 1,400 mm/sec. In some instances, the velocity may be at most 1,400 mm/sec, at most 1,300 mm/sec, at most 1,200 mm/sec, at most 1,100 mm/sec, at most 1,000 mm/sec, at most 900 mm/sec, at most 800 mm/sec, at most 700 mm/sec, at most 600 mm/sec, at most 500 mm/sec, at most 400 mm/sec, at most 300 mm/sec, at most 200 mm/sec, at most 100 mm/sec, at most 90 mm/sec, at most 80 mm/sec, at most 70 mm/sec, at most 60 mm/sec, at most 50 mm/sec, at most 40 mm/sec, at most 30 mm/sec, at most 20 mm/sec, at most 15 mm/sec, at most 10 mm/sec, or at most 1 mm/sec. Any of the lower and upper values described in this paragraph may be combined to form a range included within the present disclosure, for example, in some instances the velocity may range from about 10 mm/sec to about 1,000 mm/sec. Those of skill in the art will recognize that the velocity may have any value within this range, e.g., about 24 mm/sec.

In some instances, the cell transfection chamber (or a fluid channel) may comprise at least one pair of electrodes of the proper dimensions and/or spacing such that cells passing between the electrodes are subjected to an electric field that transiently disrupts the cell membrane and allows a transfection agent to enter the cells.

Electroporation: In some instances, the cell transfection compartment or fluid channel may comprise at least one pair of electrodes configured to perform electroporation of cells passing between the electrodes. In some instances, the cell transfection compartment may comprise one, two, three, four, five, six, seven, eight, nine, ten, or more than ten pairs of electrodes configured to perform electroporation of cells passing between the individual pairs of electrodes. In some instances, two or more pairs of electrodes may be arranged in series. In some instances, two or more pairs of electrodes may be arranged in parallel.

In some instances, the surface area of each of the electrodes in a pair of electrodes to which cells are exposed for the purpose of performing electroporation may range from about 0.001 mm² to about 100 mm². In some instances, the surface area of each electrode in a pair of electrodes may be the same. In some instances, the surface area of each electrode in a pair of electrodes may be different. In some instances, the surface area of each electrode may be at least 0.001 mm², at least 0.01 mm², at least 0.1 mm², at least 0.2 mm², at least 0.3 mm², at least 0.4 mm², at least 0.5 mm², at least 0.6 mm², at least 0.7 mm², at least 0.8 mm², at least 0.9 mm², at least 1.0 mm², at least 1.5 mm², at least 2.0 mm², at least 2.5 mm², at least 3.0 mm², at least 3.5 mm², at least 4.0 mm², at least 4.5 mm², at least 5 mm², at least 10 mm², at least 20 mm², at least 30 mm², at least 40 mm², at least 50 mm², at least 60 mm², at least 70 mm², at least 80 mm², at least 90 mm², or at least 100 mm². In some instances, the surface area of each electrode may be at most 100 mm², at most 90 mm², at most 80 mm², at most 70 mm², at most 60 mm², at most 50 mm², at most 40 mm², at most 30 mm², at most 20 mm², at most 10 mm², at most 5 mm², at most 4.5 mm², at most 4.0 mm², at most 3.5 mm², at most 3.0 mm², at most 2.5 mm², at most 2.0 mm², at most 1.5 mm², at most 1.0 mm², at most 0.9 mm², at most 0.8 mm², at most 0.7 mm², at most 0.6 mm², at most 0.5 mm², at most 0.4 mm², at most 0.3 mm², at most 0.2 mm², at most 0.1 mm², at most 0.01 mm², or at most 0.001 mm². Any of the lower and upper values described in this paragraph may be combined to form a range included within the present disclosure, for example, in some instances the surface area of each electrode may range from about 0.1 mm² to about 0.9 mm². Those of skill in the art will recognize that the surface area of each electrode may have any value within this range, e.g., about 0.66 mm².

In some instances, the separation distance between the electrodes of an electrode pair may range from about 10 μm to about 10 mm. In some instances, the separation distance between the electrodes of a first pair of electrodes and the electrodes of a second pair of electrodes may be the same. In some instances, the separation distance between the electrodes of a first pair of electrodes and the electrodes of a second pair of electrodes may be different. In some instances, the separation distance between the electrodes in a pair of electrodes may be at least 10 μm, at least 50 μm, at least 100 μm, at least 200 μm, at least 300 μm, at least 400 μm, at least 500 μm, at least 600 μm, at least 700 μm, at least 800 μm, at least 900 μm, at least 1 mm, at least 2 mm, at least 3 mm, at least 4 mm, at least 5 mm, at least 6 mm, at least 7 mm, at least 8 mm, at least 9 mm, or at least 10 mm. In some instances, the separation distance between the electrodes in a pair of electrodes may be at most 10 mm, at most 9 mm, at most 8 mm, at most 7 mm, at most 6 mm, at most 5 mm, at most 4 mm, at most 3 mm, at most 2 mm, at most 1 mm, at most 900 μm, at most 800 μm, at most 700 μm, at most 600 μm, at most 500 μm, at most 400 μm, at most 300 μm, at most 200 μm, at most 100 μm, at most 50 μm, or at most 10 μm. Any of the lower and upper values described in this paragraph may be combined to form a range included within the present disclosure, for example, in some instances the separation distance between the electrodes in a pair of electrodes may range from about 100 μm to about 2 mm. Those of skill in the art will recognize that the separation distance between the electrodes in a pair of electrodes may have any value within this range, e.g., about 785 μm.

In some instance, the voltage difference (or absolute value thereof) applied between the electrodes of a pair of electrodes may range from about 10⁻⁵ volts to about 2,000 volts. In some instances, the voltage difference applied between the electrodes of a first pair of electrodes and the voltage difference applied between the electrodes of a second pair of electrodes may be the same. In some instances, the voltage difference applied between the electrodes of a first pair of electrodes and the voltage difference applied between the electrodes of a second pair of electrodes may be different. In some instances, the voltage difference applied between the electrodes of a pair of electrodes may be at least 10⁻⁵ volts, at least 10⁻⁴ volts, at least 10⁻³ volts, at least 10⁻² volts, at least 10⁻¹ volts, at least 1 volt, at least 10 volts, at least 50 volts, at least 100 volts, at least 200 volts, at least 300 volts, at least 400 volts, at least 500 volts, at least 600 volts, at least 700 volts, at least 800 volts, at least 900 volts, at least 1,000 volts, at least 1,200 volts, at least 1,400 volts, at least 1,600 volts, at least 1,800 volts, or at least 2,000 volts. In some instances, the voltage difference applied between the electrodes of a pair of electrodes may be at most 2,000 volts, at most 1,800 volts, at most 1,600 volts, at most 1,400 volts, at most 1,200 volts, at most 1,000 volts, at most 900 volts, at most 800 volts, at most 700 volts, at most 600 volts, at most 500 volts, at most 400 volts, at most 300 volts, at most 200 volts, at most 100 volts, at most 50 volts, at most 10 volts, at most 1 volt, at most 10¹ volts, at most 10⁻² volts, at most 10⁻³ volts, at most 10⁻⁴ volts, or at most 10⁻⁵ volts. Any of the lower and upper values described in this paragraph may be combined to form a range included within the present disclosure, for example, in some instances the voltage difference applied between the electrodes of a pair of electrodes separation distance between the electrodes in a pair of electrodes may range from about 100 volts to about 800 volts. Those of skill in the art will recognize that the voltage difference applied between the electrodes of a pair of electrodes may have any value within this range, e.g., about 756 volts.

In some instances, the electric field strength (or absolute value thereof) used for electroporation of cells may range from about 0 volt/cm to about 2,000 volts/cm. In some instances, the electric field strength may be at least 0 volts/cm, at least 50 volts/cm, at least 100 volts/cm, at least 150 volts/cm, at least 200 volts/cm, at least 250 volts/cm, at least 300 volts/cm, at least 350 volts/cm, at least 400 volts/cm, at least 450 volts/cm, at least 500 volts/cm, at least 550 volts/cm, at least 600 volts/cm, at least 650 volts/cm, at least 700 volts/cm, at least 750 volts/cm, at least 800 volts/cm, at least 850 volts/cm, at least 900 volts/cm, at least 950 volts/cm, at least 1000 volts/cm, at least 1,100 volts/cm, at least 1,200 volts/cm, at least 1,300 volts/cm, at least 1,400 volts/cm, at least 1,500 volts/cm, at least 1,600 volts/cm, at least 1,700 volts/cm, at least 1,800 volts/cm, at least 1,900 volts/cm, or at least 2,000 volts/cm. In some instances, the electric field strength may be at most 2,000 volts/cm, at most 1,900 volts/cm, at most 1,800 volts/cm, at most 1,700 volts/cm, at most 1,600 volts/cm, at most 1,500 volts/cm, at most 1,400 volts/cm, at most 1,300 volts/cm, at most 1,200 volts/cm, at most 1,100 volts/cm, at most 1,000 volts/cm, at most 950 volts/cm, at most 900 volts/cm, at most 850 volts/cm, at most 800 volts/cm, at most 750 volts/cm, at most 700 volts/cm, at most 650 volts/cm, at most 600 volts/cm, at most 550 volts/cm, at most 500 volts/cm, at most 450 volts/cm, at most 400 volts/cm, at most 350 volts/cm, at most 300 volts/cm, at most 250 volts/cm, at most 200 volts/cm, at most 150 volts/cm, at most 100 volts/cm, at most 50 volts/cm, or at most 0 volts/cm. Any of the lower and upper values described in this paragraph may be combined to form a range included within the present disclosure, for example, in some instances the electric field strength may range from about 100 volts/cm to about 1,800 volts/cm. Those of skill in the art will recognize that the electric field strength may have any value within this range, e.g., about 1,875 volts/cm.

In some instances, cells may be transported through one or more pairs of electrodes at a flow rate or velocity ranging from about 0.1 mm/sec to about 100 mm/sec. In some instances, the velocity may be at least 0.1 mm/sec, at least 1 mm/sec, at least 5 mm/sec, at least 10 mm/sec, at least 20 mm/sec, at least 30 mm/sec, at least 40 mm/sec, at least 50 mm/sec, at least 60 mm/sec, at least 70 mm/sec, at least 80 mm/sec, at least 90 mm/sec, or at least 100 mm/sec. In some instances, the velocity may be at most 100 mm/sec, at most 90 mm/sec, at most 80 mm/sec, at most 70 mm/sec, at most 60 mm/sec, at most 50 mm/sec, at most 40 mm/sec, at most 30 mm/sec, at most 20 mm/sec, at most 15 mm/sec, at most 10 mm/sec, at most 5 mm/sec, at most 1 mm/sec, or at most 0.1 mm/sec. Any of the lower and upper values described in this paragraph may be combined to form a range included within the present disclosure, for example, in some instances the velocity may range from about 1 mm/sec to about 20 mm/sec. Those of skill in the art will recognize that the velocity may have any value within this range, e.g., about 17 mm/sec

Laser-induced photoporation: In some instances, a cell transfection compartment (or a fluid channel) may comprise at least one optically-transparent wall that provides optical access for a laser light beam or other light source configured to perform photoporation, e.g., laser-induced photoporation. Photoporation is based on the generation of localized transient pores in the cell membrane using focused, continuous or pulsed laser light either alone or in combination with sensitizing nanoparticles [see, e.g., R. Xiong, et al. (2016), “Laser-Assisted Photoporation: Fundamentals, Technological Advances and Applications”, Advances in Physics, 1(4):596-620]. Pores of up to several hundred nanometers in diameter can be formed in the cell membrane by a focused laser beam (typically comprising a 1-10 μm diameter focal spot) through photothermal, photomechanical and/or photochemical effects using lasers operating in the ultraviolet (UV), visible, or near-infrared (near-IR) regions of the electromagnetic spectrum. Examples of suitable laser wavelength include, but are not limited to, those listed in Table 1.

TABLE 1
Examples of laser types and wavelengths.

| | WAVELENGTH |
|---|---|
| LASER TYPE | (nanometers) |
| Xenon Chloride | 308 and 459 |
| Xenon Fluoride | 353 and 459 |
| Helium Cadmium | 325-442 |
| Rhodamine 6G | 450-650 |
| Argon | 457-528 |
| | (514.5 and 488 most used) |
| Frequency-doubled Nd: YAG | 532 |
| Helium Neon | 543, 594, 612, and 632.8 |
| Krypton | 337.5-799.3 |
| | (647.1-676.4 most used) |
| Ruby | 694.3 |
| Laser Diodes | 630-950 |
| Ti: Sapphire | 690-960 |
| Alexandrite | 720-780 |
| Nd: YAG | 1064 |

In some instances, one or more lasers may be used for performing laser-induced photoporation. In some instances, one or more of the lasers used may be continuous wave lasers. In some instances, one or more of the lasers used may be pulsed lasers. Depending on the type of laser selected and the technique used to generate pulses (e.g., mode-locked solid-state laser, Q-switched solid-state laser, or gain switched semiconductor laser), laser pulse frequencies may range from less than 1 Hz to greater than 100 GHz. Similarly, depending on the type of laser selected and the technique used to generate pulses, laser pulse widths may range from longer than 1 microsecond to fewer than 100 femtoseconds. In some instances, for example, the laser used for performing photoporation may produce pulsed light at, for example, about 1,064 nm with a pulse length of less than about 550 picoseconds.

In some instances, a laser used to perform laser-induced photoporation in the disclosed devices or cartridge may be the same as a laser used to perform photodetachment and/or photoablation, where the operating mode may be switched by adjusting one or more of the laser's average power setting, peak power setting, pulse frequency, pulse duration (pulse width), exposure time, or any combination thereof, as will be discussed in more detail below.

Needle-based poration: In some instances, a cell transfection compartment of the disclosed devices or cartridge may be configured for performing needle-based poration, e.g., where one or more microneedles are used to perforate the cell membrane and/or inject a transfection agent into the interior of the cell. In some instances, the cell transfection compartment may comprise an open region of the device or cartridge that is easily accessible. In some instances, the cell transfection compartment may comprise a compartment that is sealed with a removable lid to allow access to the interior of the compartment. In some instances, a microscope and/or micromanipulator may be used to target individual cells for transfection and to guide a microneedle.

Impalefection: In some instances, a cell transfection compartment may be configured for performing impalefection. Impalefection is a method of delivering a transfection agent to an intracellular compartment using nanomaterials, such as carbon nanofibers, carbon nanotubes, or nanowires. In some instances, needle-like nanostructures may be synthesized on a surface within the cell transfection compartment and coated with a transfection agent, e.g., plasmid DNA containing a specified gene, that is intended for intracellular delivery. Cells that are introduced into the cell transfection compartment may settle on the surface or may be pressed against the surface such that they are impaled and transfected by the nanostructures, thereby resulting in expression of the delivered gene(s).

Magnetofection: In some instances, a cell transfection compartment may be configured for performing magnetofection, a technology commercialized by OZ Biosciences, Inc. (San Diego, Calif.). Magnetofection uses magnetic fields to attract magnetic particles containing a transfection agent and draw them into the target cells. The transfection agent, e.g., a nucleic acid molecule, is associated with cationic magnetic nanoparticles; these molecular complexes are then concentrated and transported through the cell membrane and into the cells using an appropriate magnetic field to provide delivery of a high dose of the transfection agent that results in high transfection efficiency.

Sonoporation: In some instances, a cell transfection compartment may be configured for performing sonoporation (cellular sonication). Sonoporation makes use of ultrasonic mechanical vibrations and acoustic cavitation of microbubbles to modify the permeability of the cell plasma membrane and thereby allow uptake of transfection agents such as DNA molecules into the cell. The transfection efficiency of this technique can be equivalent to or better than that achieved using electroporation, although extended exposure to low-frequency (<1 MHz) ultrasound has been shown to result in rupturing and cell death, so the resulting cell viability must also be examined. In some instances, sonoporation may be implemented using commercially-available sonoporators. In some instances, sonoporation may be implemented in the disclosed devices or cartridges using integrated piezoelectric transducers.

Cell attachment and colony formation: In some instances, the disclosed devices or cartridges comprise at least one cell selection compartment which is in fluid communication with the cell transfection compartment, and into which cells are introduced following a transfection step. A plurality of individual transfected cells is introduced and allowed to attach to a surface (e.g., a “growth surface”) within the cell selection compartment and are subsequently allowed to undergo several cycles of growth and division in order to form small, clonal cell colonies. In some instances, cell doublets or triplets may inadvertently be introduced and allowed to attach to the surface within the cell selection compartment, and may be removed or destroyed (e.g., using a laser photoablation technique as will be described in more detail below) in order to ensure that all of the resulting cell colonies comprise cells of a single genotype.

In some instances, the disclosed devices or cartridges may be used to generate clonal populations of cells from adherent cell lines. In these instances, a suspension of adherent cells is introduced into the device or cartridge at an initial concentration, transfected within the cell transfection compartment, and then introduced into the cell selection compartment and allowed to settle and form attachments to one or more growth surfaces within the cell selection compartment. In some instances, a growth surface may comprise, e.g., a glass, fused-silica, silicon, or a polymer surface. In some instances, a growth surface may comprise one or more coating layers applied to an underlying surface of, e.g., glass, fused-silica, silicon, or a polymer. In some instances, the one or more coating layers may be configured to facilitate the attachment of adherent cells to the growth surface. In some instances, the one or more coating layers may be configured to facilitate the subsequent detachment of all or a portion of a clonal cell colony for subsequent genetic testing or harvesting using, e.g., an enzymatic treatment, photolysis technique, or laser photodetachment technique. Examples of suitable coatings include, but are not limited to, α-poly-lysine coatings, collagen coatings, poly-1-ornithine coatings, fibronectin coatings, laminin coatings, silane coatings, or coatings comprising an enzyme-specific substrate (e.g., the peptide sequence recognized by a specific protease) or photocleavable linker molecules. In some instances, e.g., where the disclosed devices or cartridges are used with induced pluripotent stem cells (iPSCs) derived directly from adult cells, the surface coating may comprise the Synthemax™ vitronectin coating (Corning, Inc., Corning N.Y.) and/or iMatrix-511 recombinant laminin coating (Takara Bio USA, Mountain View, Calif.).

In some instances, the disclosed devices or cartridges may be used to generate clonal populations of cells from suspension cell lines. In these instances, a suspension of cells that normally grow in solution is introduced into the device or cartridge at an initial concentration, transfected within the cell transfection compartment, and then introduced into the cell selection compartment and allowed to settle and form attachments to one or more growth surfaces within the cell selection compartment. In some instances, a growth surface may comprise, e.g., a glass, fused-silica, silicon, or a polymer surface. In some instances, a growth surface may comprise one or more coating layers applied to an underlying surface of, e.g., glass, fused-silica, silicon, or a polymer. In some instances, the one or more coating layers may be configured to facilitate the attachment of suspension cells to the growth surface. In some instances, the one or more coating layers may be configured to facilitate the subsequent detachment of all or a portion of a clonal cell colony for subsequent genetic testing or harvesting using, e.g., an enzymatic treatment, photolysis technique, or laser photodetachment technique. Examples of suitable growth surface coatings in these instances include, but are not limited to, α-poly-lysine coatings, collagen coatings, poly-1-ornithine coatings, fibronectin coatings, laminin coatings, silane coatings, or coatings comprising tethered antibodies that bind specifically to selected cell surface receptors, an enzyme-specific substrate (e.g., the peptide sequence recognized by a specific protease) or photocleavable linker molecules.

In some instances, the cell selection compartment may be seeded with transfected cells that are allowed to settle and form attachments to a surface within the compartment (e.g., a growth surface that may optionally comprise a surface coating). In some instances, the cells may be seeded at a surface density of less than or equal to 1,000 cells/mm², 900 cells/mm², 800 cells/mm², 700 cells/mm², 600 cells/mm², 500 cells/mm², 400 cells/mm², 300 cells/mm², 200 cells/mm², 100 cells/mm², 50 cells/mm², 10 cells/mm², or 5 cells/mm².

Monitoring of cell growth—imaging: As noted above, in some instances, the cell selection compartment (and/or other fluid compartments or fluid channels of the device) may be optically-transparent, thereby allowing one to monitor cell attachment, cell growth and/or the formation of clonal cell colonies using, e.g., microscopic imaging techniques. Any of a variety of microscopic imaging techniques may be used to monitor cell growth and the formation of clonal cell colonies within the cell selection compartment (or any other fluid compartments with the device). Examples include, but are not limited to bright-field, dark-field, phase contrast, fluorescence, and two-photon fluorescence imaging. In some instances, a super-resolution imaging technique may be used, e.g., super-resolution fluorescence imaging, which may allow images to be captured with a higher spatial resolution (e.g., 10-200 nm resolution) than that determined by the diffraction limit of light at the imaging wavelength. In some instances, greyscale images of cells deposited in culture plate wells may be acquired and used for cell identification and determination of cell position coordinates. In some instances, red-green-blue (RGB, or color) images of cells deposited in culture plate wells may be acquired and used for cell identification and determination of cell position coordinates. Examples of suitable imaging acquisition hardware and image processing software will be discussed in more detail below.

In some instances, imaging may be performed in a manual, semi-automated, or fully-automated manner at a frequency of at least once per day, at least twice per day, at least four times per day, at least six times per day, at least twelve times per day, at least twenty four times per day (at least once per hour), or at least once per 30 minutes.

Monitoring of cell growth—impedance measurements: In some instances, the cell selection compartment (and/or other fluid compartments or fluid channels of the device) may comprise one or more pairs of electrodes configured to monitor cell growth using electrical impedance measurements. Electrical cell-substrate impedance sensing is a method for label-free and real-time monitoring of biological cells [see, e.g., Lee, et al. (2014), “Electrical Impedance Characterization of Cell Growth on Interdigitated Microelectrode Array”, J. Nanosci. Nanotechnol. 14(10:8342-8346]. In some instances, 1, 2, 3, 4, 5, 6, 7, 8, 9, 10, or more than 10 pairs of electrodes may be used to perform electrical impedance measurements to monitor cell growth in a compartment. In some instances, the pair of electrodes may comprise, e.g., an interdigitated electrode (IDE) array comprising a set of interdigitated fingers. Lee, et al. (2014), for example, used an interdigitated electrode (IDE) array consisting of 10 fingers having a length of 1.2 mm, width of 50 μm, spacing of 50 μm, and thickness of 75 nm and measured impedance spectra of the fabricated IDE with or without cells being present in the frequency range of 100 Hz to 100 kHz using a lock-in amplifier based system. In some instances, an interdigitated pair of electrodes may comprise at least 1, 2, 3, 4, 5, 6, 7, 8, 9, 10, or more than 10 interdigitated fingers or pairs of interdigitated fingers. In some instances, the electrodes used for performing electrical impedance measurements may have a length ranging from about 0.1 mm to about 30 mm (and may have any length within this range, e.g., about 15 mm), a width ranging from about 10 μm to about 2 mm (and may have any width within this range, e.g., about 1.2 mm), and a thickness ranging from about 10 nm to about 1,000 nm (and may have any thickness within this range, e.g., about 125 nm). The electrodes may be fabricated from any of a variety of materials known to those of skill in the art including, but not limited to, platinum, gold, silver, copper, zinc, aluminum, graphene, or indium tin oxide. Impedance measurements at selected frequencies may be used to monitor cell adherence and proliferation properties that are dependent on the characteristics of specific types of cells.

Cell detection and selection: In some instances, the cell selection compartment (or any other compartment in the disclosed devices or cartridges) may be imaged for the purpose of detecting the positions of cells and/or selecting individual cells or clonal cell colonies for photodetachment, removal for testing, photoablation, and/or expansion to create one or more clonal cell populations for subsequent harvesting. In some instances, images may be viewed live by a skilled operator for identification of cells and manual control of, for example, a photodetachment or photoablation step. In some instances, images may be captured and processed using a semi-automated or fully-automated process to perform one or more of the following steps: (i) image segmentation, (ii) feature extraction, (iii) cell identification and determination of position coordinates, (iv) cell selection, and (v) transfer of cell position coordinate data for cells selected for destruction to a targeting system that, e.g., directs a laser scanning system or that controls the position of the translation stage and laser exposure to selectively detach a portion of a selected cell colony or to selectively ablate unwanted cells.

In some instances, the selection of a cell or clonal cell cluster (or a subset of cells or cell clusters) to partially detach for testing or to retain for clonal expansion is made randomly, and all other cells within the cell selection compartment are destroyed. In some instances, the selection of a cell or clonal cell cluster (or a subset of cells or cell clusters) to partially detach for testing or to retain for clonal expansion is made on the basis of selection criteria that are independent of traits or properties inherent to the cells themselves (e.g., the selecting is not based on whether the cell(s) comprises an exogenous label or an expressed reporter). For example, in some instances, a cell or clonal cell cluster is selected to be retained simply based on its location on a surface within the cell selection compartment. In some instances, a cell or clonal cell cluster (or a subset of cells or clonal cell clusters) that is closest to the center of a surface within the cell selection compartment is selected to be retained, and all other cells are ablated. In some instances, a cell or clonal cell cluster (or a subset of cells or clonal cell clusters) that is a specified distance from the center of a surface within the cell selection compartment is selected to be retained, and all other cells are ablated. In some instances, a cell or clonal cell cluster (or a subset of cells or clonal cell clusters) that is closest to a wall of the cell selection compartment is selected to be retained, and all other cells are ablated. In some instances, cell doublets, triplets, or other aggregates of cells will be ablated regardless of their position on a surface or within a cell selection compartment.

In some instances, the selection of a cell or clonal cell colony (or a subset of cells or clonal cell colonies) to retain (or destroy) is made based on selection criteria that are dependent on traits or properties inherent to the cells themselves. For example, criteria that may be used for selecting and retaining a cell (or for selecting and destroying a cell) include, but are not limited to, cell phenotype, cell genotype, cell morphology, cell size, development stage, the presence or absence of one or more specified biomarkers and/or a reporter molecule (e.g., the expression of an exogenous reporter by cells within a clonal cell colony, e.g., the presence or absence of a green fluorescent protein (GFP) signal), the number of cells within a clonal cell colony, the surface density of cells within a clonal cell colony, a growth pattern of cells within a clonal cell colony, the growth rate of cells within a clonal cell colony, a division rate of cells within a clonal cell colony, or any combination thereof.

In some instances, the selection of a cell or clonal cell cluster (or a subset of cells or clonal cell clusters) to retain (or destroy) is made based on the presence or absence of one or more biomarkers comprising cell surface receptors and ligands, e.g., G-protein coupled receptors (GPCRs), enzyme-linked receptors, ion channel-linked receptors, membrane-based receptor tyrosine kinases, membrane glycoproteins, etc. Examples of cell surface receptors and ligands that may be used as a basis for cell selection include, but are not limited to, angiotensin receptors, CD1a-e, CD3, CD4, CD6, CD8α-b, CD19, CD20, CD22, CD33, CD52, FGF receptors, growth hormone receptor, the KCNE1 ion channel, the KCNQ1 ion channel, the ATP1G1 Mg transporter, etc. (see, for example, Várady, et al. (2013), “Cell surface membrane proteins as personalized biomarkers: where we stand and where we are headed”, Biomarkers Med. 7(5), 803-819, for additional examples). In some instances, the presence or absence of one or more cell surface biomarkers may be detected using, e.g., one or more fluorescently-tagged antibodies that bind specifically to one of the biomarkers of interest.

In some instances, the selection of a cell or clonal cell cluster (or a subset of cells or clonal cell clusters) to retain (or destroy) may be made on the basis of the presence or absence of one or more biomarkers comprising genetically-engineered proteins, e.g., chimeric receptors or enzymes comprise a green fluorescence protein (GFP) domain (or a domain from any variant of GFP). In some instances, the selection of a cell or clonal cell cluster (or a subset of cells or clonal cell clusters) to retain (or destroy) may be made on the basis of the presence or absence of one or more chimeric proteins comprising a GFP domain in a cell line that has been engineered to express one or more GFP-containing proteins as part of a reporter system for detection of a change in cellular gene expression profiles (e.g., for the detection of an increase or decrease of the transcription and/or translation of a specific set of one or more genes). In some instances, the selection of a cell or clonal cell cluster (or a subset of cells or clonal cell clusters) to retain (or destroy) may be made on the basis of the presence or absence of one or more chimeric proteins comprising a GFP domain in a cell line that has been engineered to express one or more GFP-containing proteins as part of a reporter system for detection of a change in cellular gene expression profiles due to a CRISPR editing success parameter. In some instances, the CRISPR editing success parameter may comprise a Cas-dependent fluorescent moiety (e.g., a Cas9-dependent fluorescent moiety). In some instances, a deactivated Cas (dCAS) can be tagged with XFP and in combination with a guide be used to identify cells that have been edited (Ma, H. et al. (2015), “Multicolor CRISPR Labeling of Chromosomal Loci in Human Cells”, Proc. Natl. Acad. Sci. USA 112, 3002-3007).

In some instances, the selection of a cell or clonal cell cluster (or a subset of cells or clonal cell clusters) to retain (or destroy) may be made based on the presence or absence of one or more biomarkers comprising fluorescent signals that are derived from one or more fluorescent probes of cellular metabolic state. Examples of fluorescent probes that may be used to monitor cellular metabolic state include, but are not limited to, the “BioTracker” (Sigma-Aldrich, St. Louis, Mo.) series of fluorescent dyes for discriminating between live cells and dead cells, fluorescent probes for intracellular calcium²⁺ concentration (e.g., Fura 2 AM, Fura Red AM, Indo-1 AM, all from ThermoFisher Scientific, Waltham, Mass.), fluorescent probes for transmembrane potentials (e.g., FluoVolt Membrane Potential Dye, di-3-ANEPPDHQ, or bis-(1,3-dibutylbarbituric acid)trimethine oxonol (DiBAC₄(3)), all from ThermoFisher Scientific, Waltham, Mass.), etc.

In some instances, the selection of a clonal cell cluster (or subset of clonal cell clusters) growing with a cell selection compartment to retain (or destroy) may be made on the basis of partially detaching a clonal cell cluster and removing a portion or subset of the cells in the cluster from the device for testing, as will be described in more detail below.

In some instances, once one or more cells (or clonal cell clusters) have been selected for retention using any of the approaches described above, the remaining unwanted cells or clonal cell clusters may be photoablated as will be described below, and the selected cells (or clonal cell clusters) may be subjected to one or more cycles of cell growth and division to expand the clonal cell population(s). In some instances, the one or more cells or clonal cell clusters selected for retention may be transferred to another compartment, e.g., a cell expansion compartment within the device or cartridge.

Partial detachment of clonal cell colonies: In some instances, a portion or subset of cells within an individual clonal cell colony may optionally be removed from the device or cartridge for testing. In some instances, for example, a portion of the cells within an individual clonal cell colony may be detached from a surface on which they are growing using, e.g., using a laser-based photodetachment technique with a cell selection compartment that comprises at least one optically-transparent wall. Laser-based photodetachment offers a non-lethal means to dissociate adherent cells from a substrate on which they are grown without requiring chemical dissociation reagents. Adjustments to power settings, pulse-width modulation, and focal plane of the laser can be adjusted in such a way to create an energy pulse that effectively detaches the selected cells without destroying the cell membranes.

Focused laser light may, for example, be scanned across a region beneath or adjacent to one or more selected cells to detach the cells from a surface on which they are growing. In some instances, illumination by the focused laser light may result in a photothermal detachment of the one or more selected cells. In some instances, illumination by the focused laser light may result in a photomechanical detachment of the one or more selected cells. In some instances, illumination by the focused laser light may result in a photoacoustic detachment of the one or more selected cells. In some instances, the cell selection compartment may comprise one or more surface coating layers that have been specially formulated to facilitate detachment of cells growing thereon by means of a photothermal and/or photomechanical detachment mechanism. In some instances, the cell selection compartment may comprise one or more surface coating layers that comprise a photocleavable linker which tethers a cell recognition element, e.g., an antibody directed towards a cell surface receptor, to a surface within the cell selection compartment, where the cell recognition element is used to capture and tether suspension cells to a surface and where, upon illumination by focused laser light of the appropriate wavelength, the photocleavable linker is disrupted and a set of selected cells may be released from the surface.

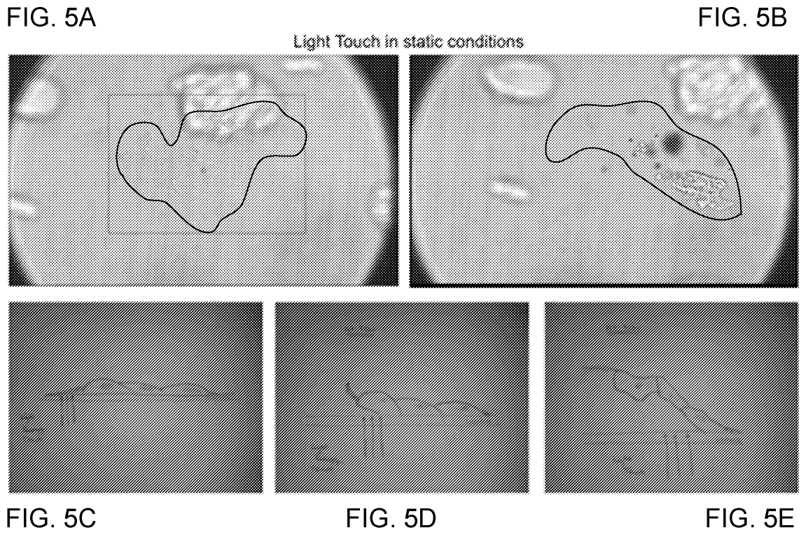

FIGS. 5A-5E illustrate the use of laser photodetachment to selectively detach cells from a substrate on which they are grown. FIG. 5A provides a micrograph of cells on a growth surface within a cell selection compartment. FIG. 5B provides a micrograph of the same surface after selectively detaching cells using pulsed laser light in a wavelength range of about 1440 nm to about 1450 nm. FIG. 5C provides an illustration of the selective detachment and removal of a subset of cells within a clonal cell cluster by irradiation with laser light. FIG. 5D provides an illustration of progressive detachment of the cells as the laser light is scanned along the surface underlying the selected cells. FIG. 5E provides an illustration of the further progressive detachment of the cells as the laser light continues to be scanned along the surface underlying the cells. The cells in FIG. 5A and FIG. 5B have been highlighted by a contour mark. The larger, out of focus objects seen in the images are imperfections in the polycarbonate created by the machining tool used to fabricate the prototype device. In FIG. 5A, one can observe a colony of cells attached to the substrate. As indicated in FIG. 5B, the cells have been detached from the substrate following selective exposure to laser light. The detached cells float above the surface and are mostly out of focus in this image.

Under static conditions, cells that have been detached may settle back down on the growth surface. In some instances, laser-based photodetachment may thus be performed in conjunction with providing a directed flow of fluid across the growth surface to direct the detached cells towards, e.g., a cell removal port through which they may be withdrawn from the device. The combination of laser-based photodetachment and flow-directed removal of detached cells allows one to remove targeted cells without risking contamination through manual intervention (e.g., through the use of media changes or chemical dissociation reagents).

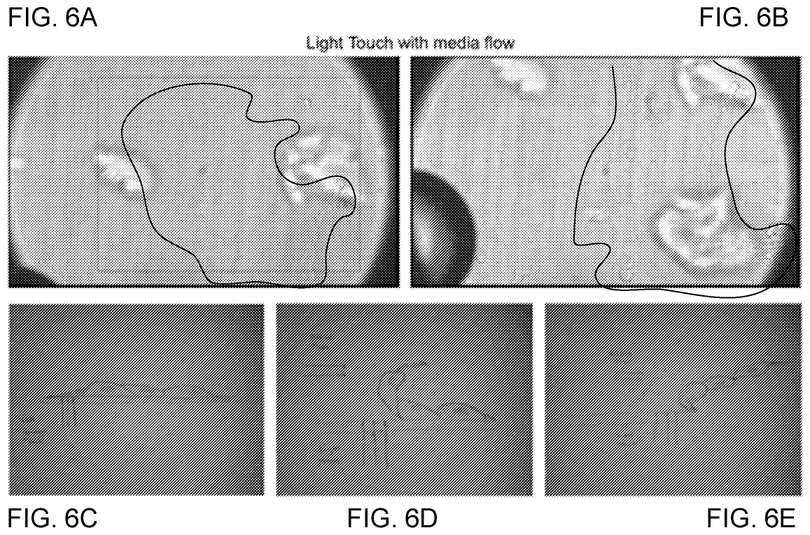

FIGS. 6A-6E illustrate the use of laser photodetachment in combination with directed fluid flow to selectively detach and remove cells from a substrate surface on which they are grown. FIG. 6A provides a micrograph of cells on a growth surface within the cell selection compartment. FIG. 6B provides a micrograph of the same surface after selectively detaching cells. FIG. 6C provides an illustration of the selective detachment and removal of a selected subset of cells within a clonal cell cluster by irradiation with laser light. FIG. 6D provides an illustration of progressive detachment of the cells as the laser light is scanned along the surface underlying the selected cells while a flow of fluid is directed across the surface. FIG. 6E provides an illustration of the further progressive detachment of the cells as the laser light continues to be scanned along the surface underlying the cells while a flow of fluid is directed across the surface. In FIGS. 6A and 6B, the same detachment process is happening as indicated in FIGS. 5A and 5B, but with a flow of buffer or culture medium directed across the surface the sheet of detached cells folds over due to the force of the fluid moving across it.

In some instances, one or more lasers may be used for performing laser-induced photodetachment. In some instances, photodetachment may be performed using lasers operating in the ultraviolet (UV), visible, or near-infrared (near-IR) regions of the electromagnetic spectrum. Examples of suitable laser wavelength include, but are not limited to, those listed in Table 1. In some instances, laser photodetachment may be performed using laser light in a wavelength range of about 1440 nm to about 1450 nm.

In some instances, one or more of the lasers used may be continuous wave lasers. In some instances, one or more of the lasers used may be pulsed lasers. Depending on the type of laser selected and the technique used to generate pulses (e.g., mode-locked solid-state laser, Q-switched solid-state laser, or gain switched semiconductor laser), laser pulse frequencies may range from less than 1 Hz to greater than 100 GHz. Similarly, depending on the type of laser selected and the technique used to generate pulses, laser pulse widths may range from longer than 1 microsecond to fewer than 100 femtoseconds.

In some instances, the same one or more lasers may be used to perform photoporation, photodetachment, and/or photoablation. In some instances, different lasers may be used to perform photoporation, photodetachment, and/or photoablation. In the case that the same laser or set of lasers is used to perform photoporation, photodetachment, and/or photoablation, the apparatus used in conjunction with the disclosed devices or cartridges may be operably switched between a photoporation operating mode, a photodetachment operating mode, and/or a photoablation operating mode by controlling laser spot size, laser spot shape, laser light intensity, laser pulse frequency, laser pulse energy, the total number of laser pulses delivered at a specified position on a surface or within the volume of at least one compartment, the position of the laser focal point relative to the surface or within the volume of the at least one compartment, or any combination thereof.

In some instances, the efficiency of laser-induced photodetachment may range from about 50% to about 100%. In some instances, the efficiency of laser-induced photodetachment may be at least 50%, at least 60%, at least 70%, at least 80%, at least 85%, at least 90%, at least 95%, at least 98%, at least 99%, or about 100%. In some instances, the efficiency of laser-induced photodetachment may be at most 100%, at most 99%, at most 98%, at most 95%, at most 90%, at most 85%, at most 80%, at most 70%, at most 60%, or at most 50%. Any of the lower and upper values described in this paragraph may be combined to form a range included within the present disclosure, for example, in some instances the efficiency of laser-induced photodetachment may range from about 60% to about 95%. Those of skill in the art will recognize that the efficiency of laser-induced photodetachment may have any value within this range, e.g., about 93%.

Cell removal and testing: In some instances, cells that have been detached from a growing surface within the cell selection compartment (or any compartment within the disclosed devices or cartridges) may optionally be removed from the device and subjected to further testing. In some instances, e.g., devices or cartridges comprising a cell selection compartment and a separate cell expansion compartment, the device or cartridge may comprise an intermediate cell removal port that is operably coupled to an outlet of the cell selection compartment and an inlet of the cell expansion compartment, such that detached cells (e.g., subsets or portions of one or more selected clonal cell clusters) may be conveniently removed from the device without risking contamination of the cell expansion compartment.

Examples of testing to which the one or more cells removed from the device may be subjected to include, but are not limited to, nucleic acid sequencing, gene expression profiling, detection of a modified RNA molecule, DNA molecule, or gene, detection of a CRISPR edited gene, a restriction site analysis of nucleic acid molecules, detection of a protein (e.g., a specific biomarker protein, a mutant protein, a reporter protein, or a genetically-engineered protein, and the like), detection of a change in an intracellular signaling pathway due to an altered protein function.

In some instances, the testing may be performed on a single cell that has been detached from a clonal cell colony and removed from the device. In some instances, the number of cells (e.g., the subset of cells that have been detached and removed from a single clonal cell colony) that are removed from the device for each clonal cell colony selected for testing may be fewer than 200 cells, fewer than 100 cells, fewer than 50 cells, fewer than 40 cells, fewer than 30 cells, fewer than 20 cells, fewer than 10 cells, or fewer than 5 cells.

Photoablation of cells with incorrect phenotypes or genotypes: In some instances, non-selected cells and/or cells having the wrong phenotype or genotype (determined, for example, by detaching a subset of cells from one or more clonal cell clusters and removing the detached cells for testing) may be destroyed so that only the selected or desired cells (or clonal cell clusters) are retained for cell expansion. In some instances, laser-based photoablation may be performed in a cell selection compartment or in any other compartment within the device or cartridge that comprises at least one optically-transparent window or wall. As noted above, the term “photoablation” as used herein and as applied to the lysis and destruction of cells may refer to a variety of related techniques in which cells are subjected to an intense beam of light to selectively destroy single cells or groups of cells.

The disruption of cells can occur via a variety of different laser light-cell interaction mechanisms that are determined primarily by the irradiance within the focal volume (Zeigler and Chiu, (2009), “Laser Selection Significantly Affects Cell Viability Following Single-Cell Nanosurgery”, Photochem. Photobiol. 85(5): 1218-1224). The mechanisms for optical disruption of cells may occur over a wide range of timescales from femtosecond (fsec) to continuous wave (cw), may comprise the use of any of a variety of lasers, and may comprise photothermal interactions, photoablation, or plasma-induced ablation (collectively referred to as “photoablation” herein). Photothermal interactions comprise the absorption of light by cells (or tags attached to said cells) that leads to local heating. Formally, photoablation can occur when absorption of a single photon by a molecule promotes an electron from a bonding to a nonbonding orbital, resulting in dissociation of the molecule. Photoablation may also result in a mechanical pressure wave radiating from the focal volume, a mechanism also known as cavitation. Plasma-induced ablation can be due to a multiphoton absorption process that results in the formation of a plasma, i.e., an ionized gas comprising positive ions and free electrons within the focal volume, which can minimize excess damage in nearby cells or tissues, and which may also lead to the formation of a cavitation bubble. Different mechanisms of laser-cell interaction may lead to significantly different outcomes for the targeted cell, e.g., to differences in cell viability. The experimental parameters that can determine which of these mechanisms dominate in cell disruption applications can be the duration of the laser pulse and its irradiance (Zeigler and Chiu, (2009), op. cit.).

In some instances of the disclosed devices, methods, and systems, the laser used for photoablation (or photoporation, or photodetachment) of cells may produce light at a peak wavelength ranging from about 220 nm (UV light) to about 1500 nm (IR light). In some instances, the peak wavelength of the laser light used for photoablation (or photoporation, or photodetachment) may be at least 220 nm, at least 250 nm, at least 300 nm, at least 350 nm, at least 400 nm, at least 450 nm, at least 500 nm, at least 550 nm, at least 600 nm, at least 650 nm, at least 700 nm, at least 750 nm, at least 800 nm, at least 850 nm, at least 900 nm, at least 950 nm, at least 1,000 nm, at least 1,100 nm, at least 1,200 nm, at least 1,300 nm, at least 1,400 nm, or at least 1,500 nm. In some instances, the peak wavelength of the laser light used for photoablation (or photoporation, or photodetachment) may be at most 1,500 nm, at most 1,400 nm, at most 1,300 nm, at most 1,200 nm, at most 1,100 nm, at most 1,000 nm, at most 950 nm, at most 900 nm, at most 850 nm, at most 800 nm, at most 750 nm, at most 700 nm, at most 650 nm, at most 600 nm, at most 550 nm, at most 500 nm, at most 450 nm, at most 400 nm, at most 350 nm, at most 300 nm, at most 250 nm, or at most 220 nm. Any of the lower and upper values described in this paragraph may be combined to form a range included within the present disclosure, for example, in some instances the peak wavelength of the laser light used for photoablation (or photoporation, or photodetachment) may range from about 1,300 nm to about 1,500 nm. Those of skill in the art will recognize that the peak wavelength of the laser light used for photoablation (or photoporation, or photodetachment) may have any value within this range, e.g., about 1,460 nm.

In some instances of the disclosed devices, methods, and systems, the laser used for photoablation (or photoporation, or photodetachment) of cells may produce light having a bandwidth (e.g., full width at half maximum (FWHM)) centered on or near the peak wavelength that ranges from about 0.0001 nm to about 10 nm, depending on peak wavelength and whether the laser is a continuous wave laser or pulsed laser. In some instances, the bandwidth may be at least 0.0001 nm, at least 0.001 nm, at least 0.01 nm, at least 0.1 nm, at least 1 nm, or at least 10 nm. In some instances, the bandwidth may be at most 10 nm, at most 1 nm, at most 0.1 nm, at most 0.01 nm, at most 0.001 nm, or at most 0.0001 nm. Any of the lower and upper values described in this paragraph may be combined to form a range included within the present disclosure, for example, in some instances the bandwidth may range from about 0.001 nm to about 1 nm. Those of skill in the art will recognize that the bandwidth of the laser light used for photoablation may have any value within this range, e.g., about 0.25 nm.

In some instances of the disclosed devices, methods, and systems, the laser used for photoablation (or photoporation, or photodetachment) of cells may produce continuous wave light, and an electro-optic modulator or electronic shutter may be used to create pulses of light of arbitrarily long duration (e.g., ranging from tens of picoseconds to seconds). In some instances of the disclosed methods and systems, the laser used for photoablation (or photoporation, or photodetachment) of cells may be a pulsed laser, and may produce light pulses having a duration ranging from about 1 femtosecond to about 100 milliseconds. In some instances, the light pulses used for photoablation may be at least 1 femtosecond, at least 1 picosecond, at least 1 nanosecond, at least 1 millisecond, at least 10 milliseconds, at least 100 milliseconds, or at least 1 second in duration. In some instances, the light pulses used for photoablation may be at most 1 second, at most 100 milliseconds, at most 10 milliseconds, at most 1 millisecond, at most 1 nanosecond, at most 1 picosecond, or at most 1 femtosecond in duration. Any of the lower and upper values described in this paragraph may be combined to form a range included within the present disclosure, for example, in some instances the light pulses used for photoablation (or photoporation, or photodetachment) may range from about 1 picosecond to about 1 nanosecond in duration. Those of skill in the art will recognize that the pulse duration of the laser light used for photoablation (or photoporation, or photodetachment) may have any value within this range, e.g., about 0.250 nanoseconds.

In some instances of the disclosed devices, methods, and systems, the laser light used for photoablation (or photoporation, or photodetachment) of cells may be pulsed at a pulse repetition frequency ranging from about 1 Hz to about 100 MHz, depending on the type of laser used. In instances, the pulse repetition frequency may be at least 1 Hz, at least 10 Hz, at least 100 Hz, at least 1 KHz, at least 10 KHz, at least 100 KHz, at least 1 MHz, at least 10 MHz, or at least 100 MHz. In some instances, the pulse repetition frequency may be at most 100 MHz, at most 10 MHz, at most 1 MHz, at most 100 KHz, at most 10 KHz, at most 1 KHz, at most 100 Hz, at most 10 Hz, or at most 1 Hz. Any of the lower and upper values described in this paragraph may be combined to form a range included within the present disclosure, for example, in some instances the pulse repetition rate may range from about 10 Hz to about 1 MHz. Those of skill in the art will recognize that the pulse repetition rate may have any value within this range, e.g., about 16.5 KHz.

In some instances, the laser light irradiance (i.e., the radiant flux (power) delivered per unit area of surface, as measured, e.g., in units of W/cm²) may range from about 0.1 W/cm² to about 10¹⁰ W/cm², depending on the type of laser used and the size of the focal spot at the sample plane. In some instances, the radiant flux delivered to the sample surface may be at least 0.1 W/cm², at least 1 W/cm², at least 10 W/cm², at least 100 W/cm², at least 1,000 W/cm², at least 10⁴ W/cm², at least 10⁵ W/cm², at least 10⁶ W/cm², at least 10⁷ W/cm², at least 10⁸ W/cm², at least 10⁹ W/cm², or at least 10¹⁰ W/cm². In some instances, the radiant flux delivered to the sample surface may be at most at most 10¹⁰ W/cm², at most 10⁹ W/cm², at most 10⁸ W/cm², at most 10⁷ W/cm², at most 10⁶ W/cm², at most 10⁵ W/cm², at most 10⁴ W/cm², at most 1,000 W/cm², at most 100 W/cm², at most 10 W/cm², at most 1 W/cm², or at most 0.1 W/cm². Any of the lower and upper values described in this paragraph may be combined to form a range included within the present disclosure, for example, in some instances the radiant flux delivered to the sample surface may range from about 10 W/cm² to about 1,000 W/cm². Those of skill in the art will recognize that the radiant flux delivered to the sample surface may have any value within this range, e.g., about 0.8 W/cm².

In some instances of the disclosed methods and systems, unwanted cells may be photoablated at a rate ranging from about 10 cells per minute to about 200 cells per minute. In some instances, unwanted cells may be photoablated at a rate of at least 10, at least 20, at least 30, at least 40, at least 50, at least 60, at least 70, at least 80, at least 90, at least 100, at least 110, at least 120, at least 130, at least 140, at least 150, at least 160, at least 170, at least 180, at least 190, or at least 200 cells per minute. In some instances, unwanted cells may be photoablated at a rate of at most 200, at most 190, at most 180, at most 170, at most 160, at most 150, at most 140, at most 130, at most 120, at most 110, at most 100, at most 90, at most 80, at most 70, at most 60, at most 50, at most 40, at most 30, at most 20, or at most 10 cells per minute. Any of the lower and upper values described in this paragraph may be combined to form a range included within the present disclosure, for example, in some instances unwanted cells may be photoablated at a rate ranging from about 50 cells per minute to about 180 cells per minute. Those of skill in the art will recognize that the photoablation rate may have any value within this range, e.g., about 64 cells per minute.

In some instances of the disclosed devices, methods and systems, the photoablation step may comprise ablating between about 80% and about 99% of the cells in a compartment, e.g., a cell selection compartment of the disclosed devices or cartridges. In some instances, at least 80%, 85%, 90%, 95%, 98%, 99%, or 99.5% of the cells on a surface or within a compartment are photoablated, where the number of cells initially on the surface or contained within the compartment is at least 5, at least 10, at least 20, at least 30, at least 40, at least 50, at least 60, at least 70, at least 80, at least 90, at least 100, at least 125, at least 150, at least 175, at least 200 cells, at least 300 cells, at least 400 cells, or at least 500 cells. Any combination of ablation percentages and number of cells initially on a surface or contained within a compartment as described above is included in the present disclosure.

In some instances of the disclosed devices, methods, and systems, the efficiency of the photoablation reaction in rendering the cells selected for destruction as non-viable ranges from about 90% to about 99.99%, or higher. In some instances, the efficiency of the photoablation step is at least 90%, at least 95%, at least 96%, at least 97%, at least 98%, at least 99%, at least 99.5%, at least 99.6%, at least 99.7%, at least 99.8%, at least 99.9%, or at least 99.99%. In some instances, the efficiency of the photoablation step is at most 99.99%, at most 99.9%, at most 99.8%, at most 99.7%, at most 99.6%, at most 99.5%, at most 99%, at most 98%, at most 97%, at most 96%, at most 95%, or at most 90%. Any of the lower and upper values described in this paragraph may be combined to form a range included within the present disclosure, for example, in some instances the efficiency of the photoablation step may range from about 95% to about 99.8%. Those of skill in the art will recognize that the efficiency of the photoablation step may have any value within this range, e.g., about 99.85%.

Further expansion of selected cells or clonal cell colonies: In some instances, the selected cells or clonal cell clusters (i.e., the remaining cells that have not been destroyed using, e.g., photoablation) are subjected to one or more cycles of cell growth and division to produce clonal cell populations. In some instances, the selected cells or clonal cell clusters may be detached from a growing surface and transferred to a separate cell expansion compartment within the device or cartridge.

In some instances, the selected cells or clonal cell clusters may be subjected to one or more cycles of cell growth and division within the same compartment used for cell selection. In either case, the cells may be supplied with fresh growth medium that is optionally stored within a growth medium reservoir that is integrated into the device or cartridge as discussed above, and that is in fluid communication (operably coupled with) an inlet of the cell selection compartment, cell expansion compartment, or other compartment used for cell expansion.

In some instances, as the selected cells or clonal cell clusters are subjected to one or more cycles of cell growth and division, spent growth medium, rinse buffers, or other fluids may be optionally transferred and stored in a waste reservoir that is integrated into the device or cartridge as discussed above, and that is in fluid communication (operably coupled with) an outlet of the cell selection compartment, cell expansion compartment, or other compartment used for cell expansion.

In some instances, the selected cells or clonal cell clusters may be subjected to at least 1, at least 2, at least 3, at least 4, at least 5, at least 10, at least 15, at least 20, at least 30, at least 40, or at least 50 cycles of cell growth and division. In some instances, the selected cells or clonal cell clusters may be subjected to at most 50, at most 40, at most 30, at most 20, at most 15, at most 10, at most 5, at most 4, at most 3, at most 2, or at most 1 cycle of cell growth and division. Any of the lower and upper values described in this paragraph may be combined to form a range included within the present disclosure, for example, in some instances the selected cells or clonal cell clusters may be subjected to from about 4 to about 8 cycles of cell growth and division. Those of skill in the art will recognize that the number of cycles of cell growth and division performed may have any value within this range, e.g., about 9 cycles.

In some instances, the selected cells or clonal cell clusters may be subjected to repeated cycles of cell growth and division until they reach a specified level of confluence on a surface on which they are grown. In some instance, for example, the selected cells or clonal cell clusters may be subjected to repeated cycles of cell growth and division until they reach at least 10% confluence, at least 20% confluence, at least 30% confluence, at least 40% confluence, at least 50% confluence, at least 60% confluence, at least 70% confluence, at least 80% confluence, or greater than 80% confluence. In some instances, the selected cells or clonal cell clusters may be subjected to repeated cycles of cell growth and division until they reach at most 80% confluence, at most 70% confluence, at most 60% confluence, at most 50% confluence, at most 40% confluence, at most 30% confluence, at most 20% confluence, or at most 10% confluence. Any of the lower and upper values described in this paragraph may be combined to form a range included within the present disclosure, for example, in some instances the selected cells or clonal cell clusters may be subjected to repeated cycles of cell growth and division until they reach from about 40% to about 85% confluence. Those of skill in the art will recognize that the selected cells or clonal cell clusters may be subjected to repeated cycles of cell growth and division until they reach any value within this range, e.g., about 87% confluence.

Monitoring of cell growth: In some instances, as the selected cells or clonal cell clusters are subjected to one or more cycles of cell growth and division, their state of growth may be monitored using imaging techniques or electrical impedance measurements as described above. Hence, in some instances, the compartment used for cell expansion may comprise at least one optically-transparent window or wall so that imaging may be performed. In some instances, the compartment used for cell expansion may comprise at least one pair of integrated electrodes (e.g., interdigitated electrodes) so that electrical impedance measurements may be performed.

Clonal cell population harvesting: In some instances, the clonal cell populations may be harvested by treating the cell expansion compartment (or any other compartment within which selected cells or clonal cell clusters are grown) with a dissociation reagent or treatment and removing the detached/released cells from the device or cartridge. Examples of dissociation reagents or treatments that may be used include, but are not limited to, mechanical detachment (e.g., shaking), trypsin treatment, trypsin-EDTA treatment, TrypLE (ThermoFisher), citric-saline buffer treatment, and the like. The detached clonal cell population to be harvested may then be removed from an outlet of the cell expansion compartment (or other compartment within which cell expansion is performed), e.g., by flowing a suitable growth medium or buffer through the compartment and out from the outlet to a collection vessel.

Other performance metrics: As noted above, the disclosed devices or cartridges, and associated methods and systems, provide for improved performance metrics in generating clonal cell populations.

In some instances, the total number of input cells required for reliable transfection and generation of a clonal cell population may be less than 10,000 cells, less than 7,500 cells, less than 5,000 cells, less than 2,500 cells, less than 1,000 cells, less than 900 cells, less than 800 cells, less than 700 cells, less than 600 cells, or less than 500 cells.

In some instances, the efficiency of performing cell transfection within the disclosed devices and cartridges may range from about 10% to about 100%. In some instances, the efficiency of performing cell transfection within the disclosed devices and cartridges may be at least 10%, at least 20%, at least 30%, at least 40%, at least 50%, at least 60%, at least 70%, at least 80%, at least 85%, at least 90%, at least 95%, at least 98%, at least 99%, or about 100%. In some instances, the efficiency of performing cell transfection within the disclosed devices and cartridges may be at most about 100%, at most 99%, at most 98%, at most 95%, at most 90%, at most 85%, at most 80%, at most 70%, at most 60%, at most 50%, at most 40%, at most 30%, at most 20%, or at most 10%. Any of the lower and upper values described in this paragraph may be combined to form a range included within the present disclosure, for example, in some instances the efficiency of performing cell transfection may range from about 40% to about 95%. Those of skill in the art will recognize that the efficiency of performing cell transfection may have any value within this range, e.g., about 87%.

In some instances, the efficiency of the selection process (e.g., the overall efficiency of selecting individual cells or clonal cell clusters, optionally detaching and removing a portion of a selected clonal cell cluster for testing, and destroying, e.g., using laser-based photoablation, all remaining non-selected and/or unwanted cells or clonal cell clusters, to yield viable cells) may range from about 10% to about 100%. In some instances, the efficiency of the selection process may be at least 10%, at least 20%, at least 30%, at least 40%, at least 50%, at least 60%, at least 70%, at least 80%, at least 85%, at least 90%, at least 95%, at least 98%, at least 99%, or about 100%. In some instances, the efficiency of the selection process may be at most about 100%, at most 99%, at most 98%, at most 95%, at most 90%, at most 85%, at most 80%, at most 70%, at most 60%, at most 50%, at most 40%, at most 30%, at most 20%, or at most 10%. Any of the lower and upper values described in this paragraph may be combined to form a range included within the present disclosure, for example, in some instances the efficiency of the selection process may range from about 70% to about 98%. Those of skill in the art will recognize that the efficiency of performing cell transfection may have any value within this range, e.g., about 92%.

In some instances, the efficiency of the cell expansion process performed within the disclosed devices and cartridge (e.g., the percentage of viable cells remaining after performing cell selection, optional detachment and removal for testing, and photoablation of non-selected and/or unwanted cells, that are then successfully grown for a specified number of cell cycles or to a specified state of confluence) may range from about 10% to about 100%. In some instances, the efficiency of the cell expansion process may be at least 10%, at least 20%, at least 30%, at least 40%, at least 50%, at least 60%, at least 70%, at least 80%, at least 85%, at least 90%, at least 95%, at least 98%, at least 99%, or about 100%. In some instances, the efficiency of the cell expansion process may be at most about 100%, at most 99%, at most 98%, at most 95%, at most 90%, at most 85%, at most 80%, at most 70%, at most 60%, at most 50%, at most 40%, at most 30%, at most 20%, or at most 10%. Any of the lower and upper values described in this paragraph may be combined to form a range included within the present disclosure, for example, in some instances the efficiency of the cell expansion process may range from about 70% to about 98%. Those of skill in the art will recognize that the efficiency of the cell expansion process may have any value within this range, e.g., about 89%.

In some instances, the overall efficiency of generating clonal populations (e.g., the combined efficiencies of performing cell transfection, cell selection, and cell expansion) within the disclosed devices or cartridges may range from about 10% to about 100%. In some instances, the overall efficiency of generating clonal populations may be at least 10%, at least 20%, at least 30%, at least 40%, at least 50%, at least 60%, at least 70%, at least 80%, at least 85%, at least 90%, at least 95%, at least 98%, at least 99%, or about 100%. In some instances, the overall efficiency of generating clonal populations may be at most about 100%, at most 99%, at most 98%, at most 95%, at most 90%, at most 85%, at most 80%, at most 70%, at most 60%, at most 50%, at most 40%, at most 30%, at most 20%, or at most 10%. Any of the lower and upper values described in this paragraph may be combined to form a range included within the present disclosure, for example, in some instances the overall efficiency of generating clonal populations may range from about 80% to about 95%. Those of skill in the art will recognize that the overall efficiency of generating clonal populations may have any value within this range, e.g., about 91%.

In some instances, the disclosed devices or cartridges (and associated methods and systems) provide for significant reductions in the amount of cell culture reagents consumed, and laboratory space required, for generating clonal cell populations. For example, in some instances, the total amount of cell culture reagents (e.g., growth media, buffers, etc.) required may be reduced compared to that used in a conventional culture plate or other culture vessel format by at least 10%, at least 20%, at least 30%, at least 40%, at least 50%, at least 60%, at least 70%, at least 80%, at least 90%, or at least 95%.

Systems and system components: Also disclosed herein are systems configured to perform the methods described above using the disclosed devices or cartridges. In some instances the disclosed systems may comprise (i) one of or more of the disclosed devices or cartridges for performing cell transfection, cell selection, and/or cell expansion, (ii) a microscope or other imaging unit (including a light source and one or more image sensors or cameras) that is configured for viewing cells on a surface or within a compartment, e.g., a cell transfection compartment, a cell selection compartment, and/or a cell expansion compartment, (iii) one or more lasers configured for performing photoporation, photodetachment, and/or photoablation (in some instances, one or more of the lasers may be optically-coupled with an imaging unit objective such that the laser and objective are capable of working in tandem to focus and deliver laser light to a specific location in a compartment), (iv) a laser targeting system (e.g., a translation stage or a laser scanning system) capable of fast and accurate positioning of individual cells at a laser focal point, or of directing focused laser light to a specific position on or near a surface or within a compartment of the disclosed devices or cartridges, (v) one or more processors, controllers, or computers, (vi) image capture and processing software for identifying cells and determining their position coordinates in each of a series of one or more compartments, (vii) laser targeting control software for controlling laser focus position, laser power, laser pulse frequency, and/or exposure time (dwell time), (viii) system control software for coordinating the fluid control, image capture, image processing, laser targeting, and laser poration, detachment, and/or ablation steps of the process, (ix) an environmental control chamber or module (e.g., an incubator) that maintains the cells within a device or cartridge under a specified set of cell culture conditions, or (xi) any combination thereof.

Microscope or imaging unit: In some instances, the disclosed systems may comprise a microscope (or other imaging unit) equipped with a camera configured to capture images of cells grown on a surface of, or within, one or more compartments. In some instances, the microscope may comprise a commercially-available microscope system, e.g., an upright, inverted, or epifluorescence microscope. In some instances, the microscope or imaging unit (or module) may comprise one or more cameras or image sensors, light sources, objective lenses, additional lenses, prisms, diffraction gratings, mirrors, optical filters, colored glass filters, narrowband interference filters, broadband interference filters, dichroic reflectors, optical filters, apertures, optical fibers, optical waveguides, and the like, or any combination thereof.

In some instances, the microscope or imaging unit of the disclosed systems may comprise an autofocus mechanism that re-focuses the microscope or imaging module on a surface (e.g., a growth surface or the bottom of a compartment) within a device or cartridge upon repositioning of the device or cartridge using a translation stage. In some instances, the microscope or imaging unit of the disclosed systems may comprise an autofocus mechanism that re-focuses the microscope or imaging module on a surface (e.g., a growth surface or the bottom of a compartment) within a device or cartridge upon redirecting a focused laser beam using, e.g., a galvanometric scanning device or micromirror array.

Any of a variety of light sources may be used to provide imaging or excitation light, including but not limited to, tungsten lamps, tungsten-halogen lamps, arc lamps, lasers, light emitting diodes (LEDs), or laser diodes. In some instances, a combination of one or more light sources, and additional optical components, e.g. lenses, filters, apertures, diaphragms, mirrors, and the like, will comprise an illumination sub-system.

Any of a variety of image sensors may be used for imaging purposes, including but not limited to, charge-coupled device (CCD) cameras or sensors, image intensified CCD cameras or sensors, CMOS image cameras or sensors, and the like. In some instances, a combination of one or more image sensors, and additional optical components, e.g. lenses, filters, apertures, diaphragms, mirrors, and the like, will comprise an imaging sub-unit (or sub-module).

Imaging mode: Any of a variety of imaging modes may be utilized in implementing the disclosed methods and systems. Examples include, but are not limited to, bright-field imaging, dark-field imaging, phase contrast imaging, fluorescence imaging, super-resolution fluorescence imaging, two-photon fluorescence imaging, and the like. In some instances, dual wavelength excitation and emission (or multi-wavelength excitation or emission) fluorescence imaging may be performed.

In some instances, each surface or compartment may be imaged in its entirety within a single image, i.e., the field-of-view (FOV) may encompass the entire surface or compartment, depending on the magnification used. In some embodiments, a series of images comprising a smaller FOV may be “tiled” or “stitched” to create a high-resolution image of the entire surface or compartment. In some instances, a series of one or more images may be acquired of all or a portion of a surface or compartment. In some instances, a series of two or more images may comprise images acquired both before and after performing, e.g., the detachment and/or ablation steps. In some instances, one or more images acquired after performing a photodetachment step may be used to confirm that the selected cell(s) have been successfully detached. In some instances, one or more images acquired after performing a photoablation step may be used to confirm that the selected cell(s) have been successfully destroyed. In some instanced, a series of images may comprise 1, 2, 3, 4, 5, 10, 15, 20, 30, 40, 50, 60, 70, 80, 90, or 100 or more images.

Image processing: In some instances of the disclosed methods and systems, image pre-processing and/or image processing may be performed in a manual, semi-automated, or fully-automated manner. In some instances, a series of one or more images may be pre-processed to, for example, correct image contrast and brightness, correct for non-uniform illumination, correct for an optical aberration (e.g., a spherical aberration, a chromatic aberration, etc.), remove noise, etc., or any combination thereof. In some instances, a series of one or more images may be processed to, for example, identify objects (e.g., cells or sub-cellular structures) within each of the images, segment each of the images to isolate the identified objects, tile segmented images to create composite images, perform feature extraction (e.g., identification and/or quantitation of object properties such as observable cellular phenotypic traits), determining the position coordinates for one or more selected cells, determining a confidence level for detachment and/or destruction of the selected cells from one or more images acquired after performing photodetachment and/or photoablation steps, or any combination thereof.

Any of a variety of image processing methods known to those of skill in the art may be used for image processing to identify objects within the images. Examples include, but are not limited to, Canny edge detection methods, Canny-Deriche edge detection methods, first-order gradient edge detection methods (e.g., the Sobel operator), second order differential edge detection methods, phase congruency (phase coherence) edge detection methods, other image segmentation algorithms (e.g., intensity thresholding, intensity clustering methods, intensity histogram-based methods, etc.), feature and pattern recognition algorithms (e.g., the generalized Hough transform for detecting arbitrary shapes, the circular Hough transform, etc.), image texture analysis methods (e.g., gray-level co-occurrence matrices), and mathematical analysis algorithms (e.g., Fourier transform, fast Fourier transform, wavelet analysis, auto-correlation, etc.), or any combination thereof.

Lasers: The disclosed systems (or apparatus) may comprise one or more lasers. As noted elsewhere, in some instances, the same one or more lasers may be used to perform photoporation, photodetachment, and/or photoablation. In some instances, different lasers may be used to perform photoporation, photodetachment, and/or photoablation. Any of a variety of lasers may be used for photoporation, photodetachment, and/or photoablation purposes. Examples include, but are not limited to, diode (or semiconductor) lasers, solid-state lasers, gas lasers, and excimer lasers. Diode lasers can provide compact, relatively low power light sources that are available for a variety of wavelengths. Solid state lasers can have lasing material distributed in a solid matrix, e.g., the ruby or neodymium-YAG (yttrium aluminum garnet) lasers. The neodymium-YAG laser can emit infrared light at 1.064 micrometers. Gas lasers, e.g., helium and helium-neon (HeNe) lasers can have a primary output of visible red light. CO₂ lasers can emit energy in the far-infrared (10.6 micrometers) and can be used for cutting hard materials. Excimer lasers can use reactive gases such as chlorine and fluorine mixed with inert gases such as argon, krypton, or xenon which, when electrically stimulated produce a pseudomolecule or dimer, and when lased produce light in the ultraviolet wavelength range.

As noted above, lasers used for photoporation, photodetachment, and/or photoablation of cells in the disclosed methods and systems may produce light at a peak wavelength ranging from about 220 nm (UV light) to about 1500 nm (IR light). In some instances, the peak wavelength of the laser light used for photoablation may be at least 220 nm, at least 250 nm, at least 300 nm, at least 350 nm, at least 400 nm, at least 450 nm, at least 500 nm, at least 550 nm, at least 600 nm, at least 650 nm, at least 700 nm, at least 750 nm, at least 800 nm, at least 850 nm, at least 900 nm, at least 950 nm, at least 1,000 nm, at least 1,100 nm, at least 1,200 nm, at least 1,300 nm, at least 1,400 nm, or at least 1,500 nm. In some instances, the peak wavelength of the laser light used for photoablation may be at most 1,500 nm, at most 1,400 nm, at most 1,300 nm, at most 1,200 nm, at most 1,100 nm, at most 1,000 nm, at most 950 nm, at most 900 nm, at most 850 nm, at most 800 nm, at most 750 nm, at most 700 nm, at most 650 nm, at most 600 n, at most 550 nm, at most 500 nm, at most 450 nm, at most 400 nm, at most 350 nm, at most 300 nm, at most 250 nm, or at most 220 nm. Any of the lower and upper values described in this paragraph may be combined to form a range included within the present disclosure, for example, in some instances the peak wavelength of the laser light used for photoablation may range from about 1,300 nm to about 1,500 nm. Those of skill in the art will recognize that the peak wavelength of the laser light used for photoporation, photodetachment, and/or photoablation may have any value within this range, e.g., about 1,460 nm.

In some instances the laser used for photoporation, photodetachment, and/or photoablation of cells in the disclosed methods and systems may produce light having a bandwidth (e.g., full width at half maximum (FWHM)) centered on or near the peak wavelength that ranges from about 0.0001 nm to about 10 nm, depending on peak wavelength and whether the laser is a continuous wave laser or pulsed laser. In some instances, the bandwidth may be at least 0.0001 nm, at least 0.001 nm, at least 0.01 nm, at least 0.1 nm, at least 1 nm, or at least 10 nm. In some instances, the bandwidth may be at most 10 nm, at most 1 nm, at most 0.1 nm, at most 0.01 nm, at most 0.001 nm, or at most 0.0001 nm. Any of the lower and upper values described in this paragraph may be combined to form a range included within the present disclosure, for example, in some instances the bandwidth may range from about 0.001 nm to about 1 nm. Those of skill in the art will recognize that the bandwidth of the laser light used for photoablation may have any value within this range, e.g., about 0.25 nm.

In some instances, the laser used for photoporation, photodetachment, and/or photoablation of cells in the disclosed methods and systems may produce continuous wave light, and an electro-optic modulator or electronic shutter may be used to create pulses of light of arbitrarily long duration (e.g., ranging from tens of picoseconds to seconds). In some instances of the disclosed methods and systems, the laser used for photoporation, photodetachment, and/or photoablation of cells may be a pulsed laser and may produce light pulses having a duration ranging from about 1 femtosecond to about 100 milliseconds. In some instances, the light pulses used for photoporation, photodetachment, and/or photoablation may be at least 1 femtosecond, at least 1 picosecond, at least 1 nanosecond, at least 1 millisecond, at least 10 milliseconds, at least 100 milliseconds, or at least 1 second in duration. In some instances, the light pulses used for photoablation may be at most 1 second, at most 100 milliseconds, at most 10 milliseconds, at most 1 millisecond, at most 1 nanosecond, at most 1 picosecond, or at most 1 femtosecond in duration. Any of the lower and upper values described in this paragraph may be combined to form a range included within the present disclosure, for example, in some instances the light pulses used for photoporation, photodetachment, and/or photoablation may range from about 1 picosecond to about 1 nanosecond in duration. Those of skill in the art will recognize that the pulse duration of the laser light used for photoporation, photodetachment, and/or photoablation may have any value within this range, e.g., about 0.250 nanoseconds.

In some instances, the laser light used for photoporation, photodetachment, and/or photoablation of cells in the disclosed methods and systems may be pulsed at a pulse repetition frequency ranging from about 1 Hz to about 100 MHz, depending on the type of laser used. In instances, the pulse repetition frequency may be at least 1 Hz, at least 10 Hz, at least 100 Hz, at least 1 KHz, at least 10 KHz, at least 100 KHz, at least 1 MHz, at least 10 MHz, or at least 100 MHz. In some instances, the pulse repetition frequency may be at most 100 MHz, at most 10 MHz, at most 1 MHz, at most 100 KHz, at most 10 KHz, at most 1 KHz, at most 100 Hz, at most 10 Hz, or at most 1 Hz. Any of the lower and upper values described in this paragraph may be combined to form a range included within the present disclosure, for example, in some instances the pulse repetition rate may range from about 10 Hz to about 1 MHz. Those of skill in the art will recognize that the pulse repetition rate may have any value within this range, e.g., about 16.5 KHz.

In some instances, the laser light irradiance (i.e., the radiant flux (power) delivered per unit area of surface, as measured, e.g., in units of W/cm²) may range from about 0.1 W/cm² to about 10¹⁰ W/cm², depending on the type of laser used and the size of the focal spot at the sample plane. In some instances, the radiant flux delivered to the sample surface may be at least 0.1 W/cm², at least 1 W/cm², at least 10 W/cm², at least 100 W/cm², at least 1,000 W/cm², at least 10⁴ W/cm², at least 10⁵ W/cm², at least 10⁶ W/cm², at least 10⁷ W/cm², at least 10⁸ W/cm², at least 10⁹ W/cm², or at least 10¹⁰ W/cm². In some instances, the radiant flux delivered to the sample surface may be at most at most 10¹⁰ W/cm², at most 10⁹ W/cm², at most 10⁸ W/cm², at most 10⁷ W/cm², at most 10⁶ W/cm², at most 10⁵ W/cm², at most 10⁴ W/cm², at most 1,000 W/cm², at most 100 W/cm², at most 10 W/cm², at most 1 W/cm², or at most 0.1 W/cm². Any of the lower and upper values described in this paragraph may be combined to form a range included within the present disclosure, for example, in some instances the radiant flux delivered to the sample surface may range from about 10 W/cm² to about 1,000 W/cm². Those of skill in the art will recognize that the radiant flux delivered to the sample surface may have any value within this range, e.g., about 0.8 W/cm².

In some instances, the disclosed systems may comprise two or more lasers operating in parallel (e.g., wherein the two laser beams are delivered to the sample plane via the same objective, but where they comprise different optical paths leading into or through the microscope or imaging unit so that they can be individually targeted to different pairs of position coordinates) such that two or more cells may be photoporated, photodetached, and/or photoablated in parallel. In some instances, the laser light provided by a single laser may be divided into two or more beams that are delivered to the sample plane via the same objective, but where different optical paths leading into or through the microscope or imaging module are used so that the divided beams can be individually targeted to different pairs of position coordinates) such that two or more cells may be photoporated, photodetached, and/or photoablated in parallel.

As noted elsewhere, in some instances the laser used to perform laser-induced photoporation in the disclosed devices or cartridge may be the same as the laser used to perform photodetachment and/or photoablation, where the operating mode may be switched by adjusting one or more of the laser's average power setting, peak power setting, pulse frequency, pulse duration (pulse width), exposure time, or any combination thereof. For example, in some instances the same laser may be used to perform photoablation and photodetachment, and may be switched between the two operating modes by, for example, reducing the power setting from about 100% of maximum (for ablation) to about 75% (for detachment) while keeping pulse width constant (e.g., about 300 μsec) and changing the rate at which the focused laser spot is scanned across the surface on which cells are grown (e.g., changing the step sizes used for ablation (e.g., X-axis step size=5 μm per unit time; Y-axis step size=1 μm per unit time) to different values for detachment (e.g., X-axis step size=Y-axis step size=15 μm per unit time), thereby reducing the effective power density delivered to the surface.

Laser targeting unit: As noted, in some instances, the disclosed systems may comprise a translation stage configured to position surfaces or compartments relative to the optical axis and/or focal plane of the microscope or imaging unit used to acquire images, and to position selected cells relative to the focal point of a laser beam. In some instances, the system may comprise a scanning mechanism, e.g., a galvanometric scanning system or a micromirror array, configured to deliver laser light to the position coordinates of one or more cells selected for photoporation, photodetachment, and/or photoablation.

In some instances, the disclosed methods and systems may utilize a high precision X-Y (or in some cases, an X-Y-Z) translation stage for re-positioning a surface or compartment in relation to the optical axis and/or focal plane of the microscope or imaging module. Suitable translation stages are commercially available from a variety of vendors, for example, Parker Hannifin. Precision translation stage systems can comprise a combination of several components including, but not limited to, linear actuators, optical encoders, servo and/or stepper motors, and motor controllers or drive units. In some cases, high precision and repeatability of stage movement can be required for the systems and methods disclosed herein in order to ensure accurate positioning of individual cells targeted for ablation. Consequently, the methods and systems disclosed herein may further comprise specifying the precision and/or repeatability with which the translation stage may position a cell in relation to the optical axis of the microscope or imaging module, or in relation to the focal spot of the laser light beam. In some instances, the precision and/or repeatability of the translation stage may range from about 0.5 μm to about 5 μm. In some instances, the precision and/or repeatability of the translation stage may be at least 0.5 μm, at least 1 μm, at least 2 μm, at least 3 μm, at least 4 μm, or at least 5 μm. In some instances, the precision and/or repeatability of the translation stage may be at most 5 μm, at most 4 μm, at most 3 μm, at most 2 μm, at most 1 μm, or at most 0.5 μm. Any of the lower and upper values described in this paragraph may be combined to form a range included within the present disclosure, for example, in some instances the precision and/or repeatability of the translation stage may range from about 1 μm to about 4 μm. Those of skill in the art will recognize that the precision and/or repeatability of the translation stage may have any value within this range, e.g., 1.25 μm.

In some instances, a galvanometric scanning system or programmable micromirror array may be used to deflect and direct one or more laser beams to specified positions on a surface or within a compartment. In some instances, the precision and/or repeatability of the translation stage may range from about 0.5 μm to about 5 μm. In some instances, the precision and/or repeatability of the galvanometric scanning system or programmable micromirror array may be at least 0.1 μm, at least 0.5 μm, at least 1 μm, at least 2 μm, at least 3 μm, at least 4 μm, or at least 5 μm. In some instances, the precision and/or repeatability of the galvanometric scanning system or programmable micromirror array may be at most 5 μm, at most 4 μm, at most 3 μm, at most 2 μm, at most 1 μm, at most 0.5 μm, or at most 0.1 μm. Any of the lower and upper values described in this paragraph may be combined to form a range included within the present disclosure, for example, in some instances the precision and/or repeatability of the galvanometric scanning system or programmable micromirror array may range from about 0.1 μm to about 2 μm. Those of skill in the art will recognize that the precision and/or repeatability of the galvanometric scanning system or programmable micromirror array may have any value within this range, e.g., 0.55 μm.

Fluidics controller: In some instances, the disclosed systems may further comprise one or more fluidics controllers configured to provide manual, semi-automated, and/or fully-automated and programmable control over the timing and/or flow rates for one or more fluids (e.g., cell culture or growth media, buffers, etc.) that are introduced into the disclosed devices or cartridges. Fluid flow may be controlled using, e.g., programmable syringe pumps, peristaltic pumps, HPLC pumps, etc.

Fluid flow rates or velocities may range from about 0.1 mm/sec to about 1,400 mm/sec. In some instances, the velocity may be at least 0.1 mm/sec, at least 1 mm/sec, at least 10 mm/sec, at least 20 mm/sec, at least 30 mm/sec, at least 40 mm/sec, at least 50 mm/sec, at least 60 mm/sec, at least 70 mm/sec, at least 80 mm/sec, at least 90 mm/sec, at least 100 mm/sec, at least 200 mm/sec, at least 300 mm/sec, at least 400 mm/sec, at least 500 mm/sec, at least 600 mm/sec, at least 700 mm/sec, at least 800 mm/sec, at least 900 mm/sec, at least 1,000 mm/sec, at least 1,100 mm/sec at least 1,200 mm/sec, at least 1,300 mm/sec, or at least 1,400 mm/sec. In some instances, the velocity may be at most 1,400 mm/sec, at most 1,300 mm/sec, at most 1,200 mm/sec, at most 1,100 mm/sec, at most 1,000 mm/sec, at most 900 mm/sec, at most 800 mm/sec, at most 700 mm/sec, at most 600 mm/sec, at most 500 mm/sec, at most 400 mm/sec, at most 300 mm/sec, at most 200 mm/sec, at most 100 mm/sec, at most 90 mm/sec, at most 80 mm/sec, at most 70 mm/sec, at most 60 mm/sec, at most 50 mm/sec, at most 40 mm/sec, at most 30 mm/sec, at most 20 mm/sec, at most 15 mm/sec, at most 10 mm/sec, at most 1 mm/sec, at most 0.1 mm/sec. Any of the lower and upper values described in this paragraph may be combined to form a range included within the present disclosure, for example, in some instances the velocity may range from about 10 mm/sec to about 1,000 mm/sec. Those of skill in the art will recognize that the velocity may have any value within this range, e.g., about 24 mm/sec.

In some instances, volumetric flow rates may range from about 1 microliter/sec to about 1 milliliter/sec. In some instances, volumetric flow rates may be at least 1 microliter/sec, at least 5 microliters/sec, at least 10 microliters/sec, at least 25 microliters/sec, at least 50 microliters/sec, at least 75 microliters/sec, at least 100 microliters/sec, at least 200 microliters/sec, at least 300 microliters/sec, at least 400 microliters/sec, at least 500 microliters/sec, at least 600 microliters/sec, at least 700 microliters/sec, at least 800 microliters/sec, at least 900 microliters/sec, or at least 1 milliliter/sec. In some instances, volumetric flow rates may be at most 1 milliliter/sec, at most 900 microliters/sec, at most 800 microliters/sec, at most 700 microliters/sec, at most 600 microliters/sec, at most 500 microliters/sec, at most 400 microliters/sec, at most 300 microliters/sec, at most 200 microliters/sec, at most 100 microliters/sec, at most 75 microliters/sec, at most 50 microliters/sec, at most 25 microliters/sec, at most 10 microliters/sec, or at most 1 microliter/sec. Any of the lower and upper values described in this paragraph may be combined to form a range included within the present disclosure, for example, in some instances the volumetric flow rate may range from about 100 microliters/sec to about 500 microliters/sec. Those of skill in the art will recognize that the volumetric flow rate may have any value within this range, e.g., about 10.35 microliters/sec.

Temperature controllers: In some embodiments, the disclosed systems may further comprise one or more temperature controllers and/or thermal interface features that are configured to maintain the one or more compartments of the disclosed devices and cartridges at a specified temperature. Examples of suitable temperature control elements include, but are not limited to, resistive heating elements, miniature infrared-emitting light sources, Peltier heating or cooling devices, heat sinks, thermistors, thermocouples, and the like. Thermal interface features will typically be fabricated from materials that are good thermal conductors (e.g., copper, gold, silver, etc.) and will typically comprise one or more flat surfaces capable of making good thermal contact with at least one external surface of the device or cartridge and/or external heating blocks or cooling blocks.

In some instances, the temperature controller may be configured to maintain one or more compartments of the disclosed devices and cartridges at a specified temperature or within a specified range of a specified temperature. In some instances, the specified temperature may be 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., or 42° C. In some instances, the temperature controller may be configured to maintain one or more compartment of the disclosed devices and cartridges to within ±0.1° C., ±0.5° C., or ±1° C. of the specified temperature.

System controllers: In some instances, the disclosed systems may comprise one or more processors, controllers, and/or computers that are configured to execute programmable, software-encoded instructions for: (i) setting and maintaining the environmental parameters (e.g., temperature, humidity, 02 concentration, CO₂ concentration, etc.) within an environmental control chamber, or within the disclosed devices and cartridges, to optimize and/or maintain cell viability during cell imaging, cell selection, and cell expansion, (ii) controlling illumination light settings (e.g., intensity and wavelength) and image acquisition (e.g., exposure time, exposure frequency, number of images acquired, etc.), (iii) controlling image pre-processing (e.g., correction of image contrast and brightness, correction for non-uniform illumination, correction for an optical aberration, removal of noise, etc., or any combination thereof) and/or image processing (identification of objects (e.g., cells or sub-cellular structures) within each of the images in a series of one or more images, segmentation of each image to isolate the identified objects, tiling of segmented images to create composite images, performing feature extraction (e.g., identification and/or quantitation of object properties such as observable cellular phenotypic traits), determining the position coordinates for one or more selected cells, determining a confidence level for the destruction of the selected cells from one or more images acquired after performing the ablation step etc., or any combination thereof), (iv) controlling the laser targeting system for performing photoporation, photodetachment, and/or photoablation of non-selected or unwanted cells (e.g., by reading the position coordinates of the selected cells and re-positioning the translation stage or re-directing a laser beam such that the selected cells are sequentially positioned within the focal spot of the laser beam) (v) controlling laser output (e.g., the intensity, pulse frequency, and/or duration of the laser light to which the selected cells are exposed), and (vi) controlling the transfer of process control data and/or image-derived data to a laboratory information management (LIMS) system, or any combination of these steps. In some instances, the system controller may further comprise control of the fluidics and/or temperature control functions.

In some instances, the one or more processors, controllers, or computers of the disclosed systems may be further configured to execute programmable, software-encoded instruction for controlling a plate-handling robotic system that moves devices or cartridges back and forth between the clonal cell population generation system and long-term cell culture incubators.

In some instances, the one or more processors of the disclosed systems may comprise a hardware processor such as a central processing unit (CPU), a graphic processing unit (GPU), a general-purpose processing unit, or computing platform. The one or more processors may be comprised of any of a variety of suitable integrated circuits (e.g., application specific integrated circuits (ASICs) designed specifically for implementing the disclosed image processing-based methods, or field-programmable gate arrays (FPGAs) to accelerate compute time, etc., and/or to facilitate deployment), microprocessors, emerging next-generation microprocessor designs (e.g., memristor-based processors), logic devices and the like. Although the disclosure is described with reference to a processor, other types of integrated circuits and logic devices may also be applicable. The processor may have any suitable data operation capability. For example, the processor may perform 512-bit, 256-bit, 128-bit, 64-bit, 32-bit, or 16-bit data operations. The one or more processors may be single core or multi core processors, or a plurality of processors configured for parallel processing.

The one or more processors or computers used to implement the disclosed methods and systems may be part of a larger computer system and/or may be operatively coupled to a computer network (or a “network”) with the aid of a communication interface to facilitate transmission of and sharing of process data and/or experimental results. The network may be a local area network, an intranet and/or extranet, an intranet and/or extranet that is in communication with the Internet, or the Internet. The network in some cases is a telecommunication and/or data network. The network may include one or more computer servers, which in some cases enables distributed computing, such as cloud computing. The network, in some cases with the aid of the computer system, may implement a peer-to-peer network, which may enable devices coupled to the computer system to behave as a client or a server.

The computer system may also include memory or memory locations (e.g., random-access memory, read-only memory, flash memory, Intel® Optane™ technology), electronic storage units (e.g., hard disks), communication interfaces (e.g., network adapters) for communicating with one or more other systems, and peripheral devices, such as cache, other memory, data storage and/or electronic display adapters. The memory, storage units, interfaces and peripheral devices may be in communication with the one or more processors, e.g., a CPU, through a communication bus, e.g., as is found on a motherboard. The storage unit(s) may be data storage unit(s) (or data repositories) for storing data.

The one or more processors, e.g., a CPU, execute a sequence of machine-readable instructions, which are embodied in a program (or “software”). The instructions are stored in a memory location. The instructions are directed to the CPU, which subsequently program or otherwise configure the CPU to implement the methods of the present disclosure. Examples of operations performed by the CPU include fetch, decode, execute, and write back. The CPU may be part of a circuit, such as an integrated circuit. One or more other components of the system may be included in the circuit. In some cases, the circuit is an application specific integrated circuit (ASIC).

In some instances, a computer system of the present disclosure may comprise a storage unit that stores files, such as drivers, libraries and saved programs. The storage unit may store user data, e.g., user-specified preferences and user-specified programs. The computer system in some cases may include one or more additional data storage units that are external to the computer system, such as located on a remote server that is in communication with the computer system through an intranet or the Internet.

Software: Some aspects of the methods and systems provided herein, such as the disclosed methods for selecting and ablating cells in a culture plate well, are implemented by way of machine-executable code (processor-executable code) stored in an electronic storage location of the computer system, such as, for example, in the memory or electronic storage unit. The machine-executable or machine-readable code is provided in the form of software. During use, the code is executed by the one or more processors. In some cases, the code is retrieved from the storage unit and stored in the memory for ready access by the one or more processors. In some situations, the electronic storage unit is precluded, and machine-executable instructions are stored in memory. The code may be pre-compiled and configured for use with a machine having one or more processors adapted to execute the code or may be compiled at run time. The code may be supplied in a programming language that is selected to enable the code to execute in a pre-compiled or as-compiled fashion.

Various aspects of the disclosed method and systems may be thought of as “products” or “articles of manufacture”, e.g., “computer program or software products”, typically in the form of machine (or processor) executable code and/or associated data that is stored in a type of machine readable medium, where the executable code comprises a plurality of instructions for controlling a computer or computer system in performing one or more of the methods disclosed herein. Machine-executable code may be stored in an optical storage unit comprising an optically readable medium such as an optical disc, CD-ROM, DVD, or Blu-Ray disc. Machine-executable code may be stored in an electronic storage unit, such as memory (e.g., read-only memory, random-access memory, flash memory) or on a hard disk. “Storage” type media include any or all of the tangible memory of the computers, processors or the like, or associated modules thereof, such as various semiconductor memory chips, optical drives, tape drives, disk drives and the like, which may provide non-transitory storage at any time for the software that encodes the methods and algorithms disclosed herein.

All or a portion of the software code may at times be communicated via the Internet or various other telecommunication networks. Such communications, for example, enable loading of the software from one computer or processor into another, for example, from a management server or host computer into the computer platform of an application server. Thus, other types of media that are used to convey the software encoded instructions include optical, electrical and electromagnetic waves, such as those used across physical interfaces between local devices, through wired and optical landline networks, and over various atmospheric links. The physical elements that carry such waves, such as wired or wireless links, optical links, or the like, are also considered media that convey the software encoded instructions for performing the methods disclosed herein. As used herein, unless restricted to non-transitory, tangible “storage” media, terms such as computer or machine “readable medium” refer to any medium that participates in providing instructions to a processor for execution.

The computer system typically includes, or may be in communication with, an electronic display for providing, for example, images captured by a machine vision system. The display is typically also capable of providing a user interface (UI). Examples of UI's include but are not limited to graphical user interfaces (GUIs), web-based user interfaces, and the like.

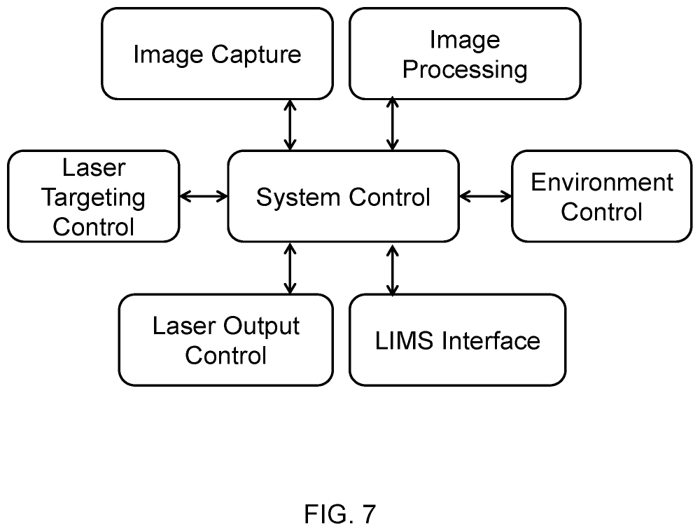

FIG. 7 provides an example of a block diagram for system control software used to control a clonal cell population generation photoablation system according to one aspect of the present disclosure. In some instances, the control software may comprise machine-readable or machine-executable instructions for communicating with and/or controlling: (i) an image acquisition module, (ii) an image processing module, (iii) a laser targeting control module, (iv) a laser output control module, (v) an environment control module, and/or (vi) a LIMS interface, or any combination of these. In some instances, the system control software may further comprise software for interfacing the clonal cell population generation systems of the present disclosure with: (vii) a robotic plate-handling system for moving devices or cartridges back and forth between the clonal cell population generation system and a long-term cell culture incubator.

In some instances, the system control software and all component modules thereof may be executed by a single processor or computer. In some instances, the system control software and one or more of the component modules may be performed on different processors or computers. In some instances, all or a portion of the system control software and/or component modules thereof may be performed by a computer network and/or cloud-based computing system.

Applications: The methods and systems disclosed herein are generally applicable to the preparation of clonal populations of cells. Examples of specific applications to which the disclosed methods and systems may be applied include, but are not limited to, generation of clonal populations of transfected cells (including clonal populations of randomly transfected cells or cells arising from targeted transfection), gene edited cells (e.g., clonal cell populations comprising a specific CRISPR edit), undifferentiated stem cells, in vitro differentiated stem cells, induced pluripotent stem cells (iPSCs), mammalian cells, plant cells, and the like.

### Exemplary Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-142 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below.

1. A cartridge comprising:
a) a first compartment configured for performing cell transfection, wherein the first compartment comprises a first inlet configured for introduction of a cell sample;
b) a second compartment configured for performing cell selection, wherein an inlet of the second compartment is operably coupled to an outlet of the first compartment, and wherein the second compartment further comprises at least one optically-transparent wall and an outlet that is operably coupled to an intermediate cell removal port; and
c) a third compartment configured for performing cell expansion, wherein an inlet of the third compartment is operably coupled to the outlet of the second compartment.
   2. A cartridge comprising:
a) a first compartment configured for performing cell transfection, wherein the first compartment comprises a first inlet configured for introduction of a cell sample;
b) a second compartment configured for performing cell selection, wherein an inlet of the second compartment is operably coupled to an outlet of the first compartment, and wherein the second compartment further comprises at least one optically-transparent wall; and
c) a third compartment configured for performing cell expansion, wherein the third compartment comprises at least one pair of electrodes configured for performing electrical impedance measurements, and wherein an inlet of the third compartment is operably coupled to the outlet of the second compartment.
   3. A cartridge comprising:
a) a first compartment configured for performing cell transfection, wherein the first compartment comprises a first inlet configured for introduction of a cell sample;
b) a second compartment configured for performing cell selection, wherein an inlet of the second compartment is operably coupled to an outlet of the first compartment, and wherein the second compartment further comprises at least one optically-transparent wall that is operably coupled to a source of laser light for performing photoablation and photodetachment; and
c) a third compartment configured for performing cell expansion, wherein an inlet of the third compartment is operably coupled to the outlet of the second fluid compartment.
   4. A system comprising:

a cartridge comprising at least one compartment configured for performing one or more of: cell transfection, cell selection and/or cell expansion, wherein the cartridge comprises an inlet configured for introduction of a cell sample and the at least one compartment comprises an optically-transparent wall.

5. The system of embodiment 4 further comprising a light source to facilitate performance of a photodetachment process and/or a photoablation process.
6. The system of embodiment 4 or 5, wherein the cartridge comprises at least one compartment for cell selection and at least one compartment for cell expansion.
7. The system of any one of embodiments 4 to 6, wherein the cartridge comprises a plurality of compartments for cell selection and a plurality of compartments for cell expansion.
8. The system of any one of embodiments 4 to 7, wherein the cartridge comprises at least four compartments for cell selection, at least eight compartments for cell selection, at least sixteen compartments for cell selection, at least thirty two compartments for cell selection, at least sixty four compartments for cell selection, or at least ninety six compartments for cell selection.
9. The system of any one of embodiments 4 to 8, wherein the cartridge comprises at least four compartments for cell expansion, at least eight compartments for cell expansion, at least sixteen compartments for cell expansion, at least thirty two compartments for cell expansion, at least sixty four compartments for cell expansion, or at least ninety six compartments for cell expansion.
10. The system of any one of embodiments 4 to 9, wherein the cartridge comprises at least one compartment for cell transfection.
11. The system of any one of embodiments 4 to 9, wherein the cartridge does not comprise a compartment for cell transfection.
12. The cartridge or system of any one of embodiments 1 to 10, wherein the first compartment or at least one compartment further comprises at least one of: (i) a second inlet configured for introduction of a transfection agent, (ii) a constricted flow path, (iii) a pair of electrodes in electrical contact with and positioned on opposing surfaces of the first compartment or at least one compartment, and (iv) an optically-transparent wall.
13. The cartridge or system of embodiment 12, wherein the pair of electrodes are fabricated from platinum, gold, silver, copper, zinc, aluminum, graphene, or indium tin oxide.
14. The cartridge or system of any one of embodiments 5 to 13, wherein the optically-transparent wall of the first compartment or of at least one compartment is transparent in the ultraviolet, visible, or near-infrared regions of the electromagnetic spectrum, or any combination thereof.
15. The cartridge or system of any one of embodiments 5 to 14, wherein the cell selection compartment comprises a pattern of indentations on an inner surface and/or a pattern of a substrate on an inner surface.
16. The cartridge or system of any one of embodiments 5 to 15, wherein the cell expansion compartment comprises a pattern of indentations on an inner surface and/or a pattern of a substrate on an inner surface.
17. The cartridge or system of embodiment 15 or 16, wherein the substrate is a protein substrate.
18. The cartridge or system of any one of embodiments 15 to 17, wherein the pattern of indentations and/or the pattern of a substrate are configured to prevent cell migration within the compartments.
19. The cartridge or system of any one of embodiments 1 to 18, wherein a volume of the second compartment or of at least one compartment is between about 1 microliter and about 10 milliliters.
20. The cartridge or system of any one of embodiments 1 to 19, wherein the optically-transparent wall of the second compartment or of at least one compartment is transparent in the ultraviolet, visible, or near-infrared regions of the electromagnetic spectrum, or any combination thereof.
21. The cartridge or system of any one of embodiments 1 to 20, wherein the optically-transparent wall of the second compartment or of at least one compartment is transparent in the range from about 1440 nm to about 1450 nm.
22. The cartridge or system of any one of embodiments 1 to 21, wherein a wall of the second compartment or of at least one compartment comprises a surface coating or surface treatment to facilitate attachment of adherent cells.
23. The cartridge or system of any one of embodiments 1 to 22, wherein a wall of the second compartment or of at least one compartment comprises a surface coating or surface treatment to facilitate attachment of suspension cells.
24. The cartridge or system of embodiment 22 or embodiment 23, wherein the surface coating is selected from the group consisting of an α-poly-lysine coating, a collagen coating, a poly-1-ornithine, a fibronectin coating, a laminin coating, a Synthemax™ vitronectin coating, an iMatrix-511 recombinant laminin coating, and any combination thereof.
25. The cartridge or system of embodiment 22 or embodiment 23, wherein the surface treatment comprises a plasma treatment, a UV treatment, an ozone treatment, or any combination thereof.
26. The cartridge or system of any one of embodiments 22 to 25, wherein the wall of the second compartment or of at least one compartment that comprises the surface coating or surface treatment is the optically-transparent wall.
27. The cartridge or system of any one of embodiments 1 to 26, wherein the second compartment (or at least one compartment) comprises a chamber having no physical barriers, flow constrictions, or partitions positioned therein.
28. The cartridge or system of any one of embodiments 1 to 27, wherein a longest dimension of the third compartment (or at least one compartment) is between about 1 centimeter and about 20 centimeters.
29. The cartridge or system of any one of embodiments 1 to 28, wherein a volume of the third compartment (or at least one compartment) is between about 1 microliter and about 1 milliliter.
30. The cartridge or system of any one of embodiments 1 to 29, wherein the third compartment (or at least one compartment) further comprises at least one optically-transparent wall.
31. The cartridge or system of embodiment 30, wherein the optically-transparent wall is transparent in the ultraviolet, visible, or near-infrared regions of the electromagnetic spectrum, or any combination thereof.
32. The cartridge or system of any one of embodiments 1 to 31, wherein the third compartment (or at least one compartment) further comprises at least one pair of electrodes configured for performing electrical impedance measurements.
33. The cartridge or system of any one of embodiments 1 to 32, further comprising a fourth compartment (or at least one compartment) configured for storing a cell growth medium.
34. The cartridge or system of any one of embodiments 1 to 33, further comprising a fifth compartment (or at least one compartment) configured for storing waste.
35. The cartridge or system of any one of embodiment 33 or embodiment 34, wherein the fourth or fifth compartment (or at least one compartment) further comprises a gas permeable membrane.
36. The cartridge or system of any one of embodiments 1 to 35, wherein the inlet of the second compartment (or at least one compartment) is operably coupled to a source of a reagent that facilitates detachment of cells from a surface within the second compartment (or the at least one compartment).
37. The cartridge or system of any one of embodiments 1 to 36, wherein the inlet of the third compartment (or at least one compartment) is operably coupled to a source of a reagent that facilitates detachment of cells from a surface within the third compartment (or an at least second compartment).
38. The cartridge of any one of embodiments 1 to 37, wherein the cartridge is fabricated from glass, fused-silica, silicon, polydimethylsiloxane (PDMS), polymethylmethacrylate (PMMA), polycarbonate (PC), polypropylene (PP), polyethylene (PE), high density polyethylene (HDPE), polyimide (PI), cyclic olefin polymers (COP), cyclic olefin copolymers (COC), polyethylene terephthalate (PET), polystyrene (PS), epoxy resin, ceramic, metal, or any combination thereof.
39. The cartridge or system of any one of embodiments 1 to 38, wherein the outlet of the second compartment (or at least one compartment) is operably coupled to the cell removal port and the inlet of the third compartment (or an at least second compartment) using a valve.
40. The cartridge or system of any one of embodiments 33 to 40, wherein the inlet of the third compartment (or at least one compartment) is operably coupled to the outlet of the second compartment (or an at least second compartment) and the outlet of the fourth compartment (or an at least third compartment) using a valve.
41. The cartridge or system of embodiment 39 or embodiment 40, wherein the valve is a programmable three-way valve.
42. The cartridge or system of any one of embodiments 1 to 41, wherein the microfluidic cartridge has a footprint that complies with American National Standards Institute (ANSI) Standard Number SLAS 4-2004 (R2012).
43. The cartridge or system of any of embodiments 1 to 41, wherein the microfluidic cartridge has a footprint that is 127.76 mm±0.5 mm in length and 85.48 mm±0.5 mm in width.
44. A method for producing a clonal population of transfected cells, the method comprising:
a) providing a cartridge, wherein the cartridge comprises at least one compartment configured for performing cell transfection, cell selection, cell expansion, or any combination thereof, and wherein at least one compartment comprises an optically-transparent wall;
b) introducing a cell sample into the at least one compartment;
c) transfecting the cell sample with one or more transfection agents;
d) selecting at least one clonal cell colony derived from the transfected cell sample;
e) performing photoablation to destroy all clonal cell colonies except the at least one clonal cell colony selected in (d); and
f) subjecting the at least one clonal cell colony selected in (d) to one or more cycles of cell division and growth to produce a clonal population of transfected cells.
   45. The method of embodiment 44, further comprising detaching a first subset of cells from the at least one clonal cell colony selected in (d) and removing them from the cartridge for testing.
   46. The method of embodiment 45, further comprising performing photoablation to destroy all remaining clonal cell colonies except a subset of those for which a first subset of cells was detached and subjected to testing.
   47. The method of any one of embodiments 44 to 46, wherein the cell sample comprises adherent cells.
   48. The method of any one of embodiments 44 to 46, wherein the cell sample comprises suspension cells.
   49. The method of any one of embodiments 44 to 48, wherein the cell sample comprises mammalian cells.
   50. The method of embodiment 49, wherein the mammalian cells are human cells.
   51. The method of any one of embodiments 44 to 50, wherein the number of cells in the cell sample is less than 10,000.
   52. The method of any one of embodiments 44 to 51, wherein the number of cells in the cell sample is less than 5,000.
   53. The method of any one of embodiments 44 to 52, wherein the number of cells in the cell sample is less than 1,000.
   54. The method of any one of embodiments 44 to 53, wherein the number of cells in the cell sample is less than 500.
   55. The method of any one of embodiments 44 to 54, wherein the one or more transfection agents comprise one or more types of DNA molecule, RNA molecule, oligonucleotide, aptamer, non-plasmid nucleic acid molecule, ribonucleoprotein (RNP), plasmid, viral vector, cosmid, artificial chromosome, or any combination thereof.
   56. The method of any one of embodiments 44 to 55, wherein the transfecting performed in (c) comprises chemical transfection, mechanical transfection (squeezing), electroporation, laser-induced photoporation, needle-based poration, impalefection, magnetofection, sonoporation, or any combination thereof.
   57. The method of any one of embodiments 44 to 56, wherein the clonal cell colonies derived from the transfected cell sample are grown by seeding a surface of at least one compartment with transfected cells at a cell surface density of less than or equal to 50 cells/mm².
   58. The method of any one of embodiments 44 to 57, wherein the clonal cell colonies derived from the transfected cell sample are grown by seeding a surface of at least one compartment with transfected cells at a cell surface density of less than or equal to 10 cells/mm².
   59. The method of any one of embodiments 44 to 58, wherein the clonal cell colonies derived from the transfected cell sample are grown by seeding a surface of at least one compartment with transfected cells at a cell surface density of less than or equal to 5 cells/mm².
   60. The method of any one of embodiments 44 to 59, wherein after seeding at least one compartment with transfected cells, any clusters of cells comprising two or more cells are destroyed using a photoablation step prior to allowing single cells to form clonal colonies.
   61. The method of any one of embodiments 44 to 60, wherein the selecting in (d) comprises randomly-selecting one or more clonal cell colonies.
   62. The method of any one of embodiments 44 to 60, wherein the selecting in (d) comprises selecting the at least one clonal cell colony based on a position on an interior surface of the at least one compartment.
   63. The method of any one of embodiments 44 to 60, wherein the selecting in (d) is based on a number of cells within the at least one clonal cell colony, a morphology of cells within the at least one clonal cell colony, a surface density of cells within the at least one clonal cell colony, a growth pattern of cells within the at least one clonal cell colony, a growth rate of cells within the at least one clonal cell colony, a division rate of cells within the at least one clonal cell colony, expression of an exogenous reporter by cells within the at least one clonal cell colony, or any combination thereof.
   64. The method of any one of embodiments 44 to 63, wherein the selecting in (d) is based on imaging a surface on which, or a volume within which, the at least one clonal cell colony is grown.
   65. The method of embodiment 64, wherein the imaging comprises performing bright-field imaging, dark-field imaging, phase contrast imaging, fluorescence imaging, or any combination thereof.
   66. The method of embodiment 64 or embodiment 65, wherein acquired images are processed using automated image analysis software.
   67. The method of any one of embodiments 64 to 66, wherein a field-of-view of an imaging system used to perform the imaging is smaller than an area of the surface or volume, and wherein the imaging comprises acquiring two or more individual images that collectively cover all or a portion of the area of the surface or volume.
   68. The method of any one of embodiments 64 to 67, wherein the imaging is performed at a frequency of at least once per day.
   69. The method of any one of embodiments 64 to 68, wherein the imaging is performed at a frequency of at least once per hour.
   70. The method of any one of embodiments 64 to 69, wherein the selecting in (d) is performed automatically based on automated image analysis of one or more images.
   71. The method of any one of embodiments 44 to 70, wherein a wall of at least one compartment comprises a surface coating or surface treatment to facilitate attachment of adherent cells.
   72. The method of any one of embodiments 44 to 71, wherein a wall of at least one compartment comprises a surface coating or surface treatment to facilitate attachment of suspension cells.
   73. The method of embodiment 71 or embodiment 72, wherein the surface coating is selected from the group consisting of an α-poly-lysine coating, a collagen coating, a poly-1-ornithine, a fibronectin coating, a laminin coating, a Synthemax™ vitronectin coating, an iMatrix-511 recombinant laminin coating, and any combination thereof.
   74. The method of embodiment 71 or embodiment 72, wherein the surface treatment comprises a plasma treatment, a UV treatment, an ozone treatment, or any combination thereof.
   75. The method of any one of embodiments 71 to 74, wherein the wall of the at least one compartment that comprises the surface coating or surface treatment is the optically-transparent wall.
   76. The method of any one of embodiments 45 to 75, wherein the first subset of cells is detached using laser photodetachment.
   77. The method of embodiment 76, further comprising subjecting the first subset of cells to a flow of liquid directed across a surface on which the at least one clonal cell colony is grown while a region of the surface beneath or adjacent to the at least one clonal cell colony is illuminated with laser light.
   78. The method of embodiment 76 or embodiment 77, wherein illumination with laser light results in cleavage of a photocleavable linker used to tether cells to the wall of the at least one compartment.
   79. The method of embodiment 76 or embodiment 77, wherein illumination with laser light results in a photothermal detachment of the first subset of cells.
   80. The method of embodiment 76 or embodiment 77, wherein illumination with laser light results in a photomechanical detachment of the one or more selected cells.
   81. The method of embodiment 76 or embodiment 77, wherein illumination with laser light results in a photoacoustic detachment of the one or more selected cells.
   82. The method of any one of embodiments 76 to 81, wherein the laser photodetachment is performed using laser light in a wavelength range of about 1440 nm to about 1450 nm.
   83. The method of any one of embodiments 76 to 82, wherein an efficiency of photodetaching the first subset of cells is at least 80%.
   84. The method of any one of embodiments 76 to 83, wherein an efficiency of photodetaching the first subset of cells is at least 90%.
   85. The method of any one of embodiments 76 to 84, wherein an efficiency of photodetaching the first subset of cells is at least 95%.
   86. The method of any one of embodiments 45 to 85, wherein the first subset of cells comprises fewer than 100 cells.
   87. The method of any one of embodiments 45 to 86, wherein the first subset of cells comprises fewer than 50 cells.
   88. The method of any one of embodiments 45 to 87, wherein the first subset of cells comprises fewer than 10 cells.
   89. The method of any one of embodiments 45 to 88, wherein the first subset of cells comprises a single cell.
   90. The method of any one of embodiments 45 to 89, wherein the testing comprises nucleic acid sequencing.
   91. The method of any one of embodiments 45 to 89, wherein the testing comprises gene expression profiling.
   92. The method of any one of embodiments 45 to 89, wherein the testing comprises detection of a modified gene.
   93. The method of any one of embodiments 45 to 89, wherein the testing comprises detection of a CRISPR edited gene.
   94. The method of any one of embodiments 45 to 89, wherein the testing comprises performing a restriction site analysis of nucleic acid molecules.
   95. The method of any one of embodiments 45 to 89, wherein the testing comprises detection of a protein.
   96. The method of embodiment 95, wherein the protein comprises a mutant protein, a reporter protein, or a genetically-engineered protein.
   97. The method of any one of embodiments 45 to 89, wherein the testing comprises detection of a change in an intracellular signaling pathway due to an altered protein function.
   98. The method of any one of embodiments 44 to 97, wherein the photoablation is performed using laser light in a wavelength range of about 1440 nm to about 1450 nm.
   99. The method of any one of embodiments 44 to 98, wherein an efficiency of photoablation is at least 80%.
   100. The method of any one of embodiments 44 to 99, wherein an efficiency of photoablation is at least 90%.
   101. The method of any one of embodiments 44 to 100, wherein an efficiency of photoablation is at least 95%.
   102. The method of any one of embodiments 44 to 101, wherein growth of the clonal population of transfected cells is monitored using electrical impedance measurements.
   103. The method of any one of embodiments 44 to 102, further comprising harvesting the clonal population of transfected cells after a specified number of cell division and growth cycles.
   104. The method of any one of embodiments 44 to 103, further comprising harvesting the clonal population of transfected cells after they have reached at least 70% confluency in the at least one compartment.
   105. An apparatus comprising:
a) a cartridge, wherein the cartridge comprises at least one compartment configured for performing cell transfection, cell selection, cell expansion, or any combination thereof, wherein at least one compartment comprises an optically-transparent wall that is operably coupled to a source of laser light for performing photoablation and photodetachment; and
b) a controller.
   106. The apparatus of embodiment 105, wherein the controller is configured to perform at least one of:
i) controlling timing and flowrate for one or more fluids flowing through the cartridge;
ii) performing manual, semi-automated, or fully-automated image processing of images acquired by an imaging unit and, based on data derived from the processed images, selecting a first subset of cells for laser-based photodetachment and a second subset of cells for laser-based photoablation; and
iii) controlling laser operating parameters for one or more lasers and a laser targeting unit such that the first subset of cells is photodetached and the second subset of cells is photoablated.
   107. The apparatus of embodiment 106, wherein the first subset of cells and the second subset of cells are both derived from a single clonal cell colony.
   108. The apparatus of embodiment 106, wherein the laser targeting unit comprises a translation stage configured to accurately position cells growing on a surface within, or within a volume of, the at least one compartment at, or adjacent to, a laser focal point on an object plane of the imaging unit.
   109. The apparatus of embodiment 106, wherein the laser targeting unit comprises a scanning mechanism configured to direct focused laser light at, or adjacent to, the positions of one or more cells growing on a surface within, or within a volume of, the at least one compartment.
   110. The apparatus of any one of embodiments 105 to 109, wherein cell transfection is performed in a first compartment, and cell selection and cell expansion are performed in a second compartment.
   111. The apparatus of any one of embodiments 105 to 109, wherein cell transfection, cell selection, and cell expansion are each performed in a separate compartment.
   112. The apparatus of any one of embodiments 105 to 109, wherein cell transfection, cell selection, and cell expansion are all performed in the same compartment.
   113. The apparatus of any one of embodiments 106 to 112, wherein the imaging unit is configured to perform bright-field imaging, dark-field imaging, phase contrast imaging, fluorescence imaging, or any combination thereof.
   114. The apparatus of any one of embodiments 106 to 113, wherein a field-of-view of the imaging unit is smaller than an area of a surface of, or volume within, the at least one compartment on or within which cells are grown or attached, and wherein the imaging unit is configured to acquire and tile two or more individual images that collectively cover all or a portion of the area of the surface or volume.
   115. The apparatus of any one of embodiments 106 to 114, wherein the imaging unit is configured to acquire images at a frequency of at least once per day.
   116. The apparatus of any one of embodiments 106 to 115, wherein the imaging unit is configured to acquire images at a frequency of at least once per hour.
   117. The apparatus of any one of embodiments 106 to 116, wherein the selecting in (ii) comprises randomly-selecting one or more clonal cell colonies.
   118. The apparatus of any one of embodiments 106 to 116, wherein the selecting in (ii) comprises selecting one or more clonal cell colonies based on a position on a surface of the at least one compartment.
   119. The apparatus of any one of embodiments 106 to 116, wherein the selecting in (ii) is based on a number of cells within a clonal cell colony, a morphology of cells within a clonal cell colony, a surface density of cells within a clonal cell colony, a growth pattern of cells within a clonal cell colony, a growth rate of cells within a clonal cell colony, a division rate of cells within a clonal cell colony, expression of an exogenous reporter by cells within a clonal cell colony, or any combination thereof.
   120. The apparatus of any one of embodiments 106 to 119, wherein the same laser is used to perform photoablation and photodetachment.
   121. The apparatus of any one of embodiments 106 to 120, wherein the one or more lasers used for photodetachment and photoablation are optically coupled to the imaging system through an objective lens used for imaging.
   122. The apparatus of any one of embodiments 106 to 121, wherein the one or more lasers used to perform photodetachment and photoablation comprise at least one pulsed laser.
   123. The apparatus of any one of embodiments 106 to 122, wherein the one or more lasers used to perform photodetachment and photoablation comprise at least one infrared laser.
   124. The apparatus of any one of embodiments 105 to 123, wherein the apparatus is operably switched between a photodetachment operating mode and a photoablation operating mode by controlling a laser spot size, a laser spot shape, a laser light intensity, a laser pulse frequency, a laser pulse energy, a total number of laser pulses delivered at a specified position on the surface or within the volume of the at least one compartment, a position of a laser focal point relative to the surface or within the volume of the at least one compartment, or any combination thereof.
   125. The apparatus of any one of embodiments 106 to 124, wherein the controller is further configured to subject the first subset of cells to a flow of liquid directed across the surface within the at least one compartment while a region of the surface beneath or adjacent to the first subset of cells is illuminated with laser light.
   126. The apparatus of any one of embodiments 106 to 125, wherein an efficiency of photodetaching the first subset of cells is at least 80%.
   127. The apparatus of any one of embodiments 106 to 126, wherein an efficiency of photodetaching the first subset of cells is at least 90%.
   128. The apparatus of any one of embodiments 106 to 127, wherein an efficiency of photodetaching the first subset of cells is at least 95%.
   129. The apparatus of any one of embodiments 106 to 128, wherein the second subset of cells is photoablated with an efficiency of greater than 90%.
   130. The apparatus of any one of embodiments 106 to 129, wherein the second subset of cells is photoablated with an efficiency of greater than 95%.
   131. The apparatus of any one of embodiments 106 to 130, wherein the second subset of cells is photoablated with an efficiency of greater than 99%.
   132. The apparatus of any one of embodiments 106 to 131, wherein the second subset of cells is photoablated with an efficiency of greater than 99.9%.
   133. The apparatus of any one of embodiments 106 to 132, wherein the one or more lasers are further configured to perform laser-based photoporation of cells in the at least one compartment.
   134. The apparatus of any one of embodiments 105 to 133, wherein at least one compartment of the cartridge is configured to perform chemical transfection, mechanical transfection (squeezing), electroporation, laser-induced photoporation, needle-based poration, impalefection, magnetofection, sonoporation, or any combination thereof.
   135. The apparatus of any one of embodiments 105 to 134, further comprising an incubator unit for maintaining the at least one compartment of the cartridge under a specified set of growth conditions.
   136. A non-transitory computer-readable medium storing a set of instructions which, when executed by a processor, cause a processor-controlled system to perform steps comprising:
a) controlling timing and flowrate for one or more fluids flowing through a cartridge comprising at least one compartment configured to perform cell transfection, cell selection, cell expansion, or any combination thereof;
b) performing image processing of images acquired by an imaging unit configured to image a surface or volume within the at least one compartment and, based on data derived from the processed images, selecting: (i) a first subset of cells growing on a surface of or in a volume within the at least one compartment for laser-based photodetachment and (ii) a second subset of cells growing on a surface of or in a volume within the at least one compartment for laser-based photoablation; and
c) controlling one or more operating parameters of one or more lasers and a laser targeting unit such that the first subset of cells is photodetached and the second subset of cells is photoablated.
   137. The non-transitory computer-readable medium of embodiment 136, wherein the selecting in (b) comprises randomly selecting one or more clonal cell colonies.
   138. The non-transitory computer-readable medium of embodiment 136 or embodiment 137, wherein the selecting in (b) comprises selecting one or more clonal cell colonies based on a position on an interior surface of the cell selection compartment.
   139. The non-transitory computer-readable medium of any one of embodiments 136 to 138, wherein the selecting in (b) is based on a number of cells within a clonal cell colony, a morphology of cells within a clonal cell colony, a surface density of cells within a clonal cell colony, a growth pattern of cells within a clonal cell colony, a growth rate of cells within a clonal cell colony, a division rate of cells within a clonal cell colony, expression of an exogenous reporter by cells within a clonal cell colony, or any combination thereof.
   140. The non-transitory computer-readable medium of any one of embodiments 136 to 139, wherein the processor-controlled system is operably switched between a photodetachment operating mode and a photoablation operating mode by: controlling a laser spot size, a laser spot shape, a laser light intensity, a laser pulse frequency, a laser pulse energy, a total number of laser pulses delivered at a specified position on a surface within the at least one compartment, a position of a laser focal point relative to the surface within the at least one compartment, a position of a laser focal point within the volume of the at least one compartment, or any combination thereof.
   141. The non-transitory computer-readable medium of any one of embodiments 136 to 140, further comprising instructions for delivering the photodetached first subset of cells to an outlet port of the cartridge for testing.
   142. The non-transitory computer-readable medium of any one of embodiments 136 to 141, further comprising instructions for performing photodetachment of a third subset of cells following photoablation of the second subset of cells and delivering the detached third subset of cells to an at least second compartment configured to perform cell expansion.

### EXAMPLES

These examples are provided for illustrative purposes only and not to limit the scope of the claims provided herein.

### Example 1—Prophetic Example—Preparation of a Clonal Population of Transfected Cells

The disclosed devices provide for simplified transfection of cells and generation of clonal populations therefrom in a format that greatly reduces cell culture reagent consumptions and laboratory space requirements. Referring to FIG. 1, a dilute cell suspension is mixed with a transfection agent in solution and introduced to the device through a fluid inlet. Within the device, the mixture of cells and transfection agent are passed through a transfection compartment. In the example of FIG. 1, the transfection compartment comprises at least one pair of electrodes and is configured to perform electroporation of cell membranes, thereby transiently disrupting the cell membranes and allowing the transfection agent to enter the cells.

Following transfection, the cells are transferred to a cell selection compartment where they settle and form attachments to a lower surface within the compartment. In some instances, the surface may comprise one or more coating layers that are formulated to facilitate attachment and adhesion of the cells to the coated surface. The positions of attached single cells (or of small clonal clusters of cells) may be identified using imaging of the surface—either manually or through the use of automated image processing software—and their initial growth to form clonal cell clusters comprising several cells is monitored. Optionally, cell doublets, triplets, or higher-order aggregates of cells that settle on the surface prior to growth may be destroyed, e.g., using a laser photoablation technique, provided that the cell selection compartment comprises at least one optically-transparent wall or window.

After reaching a specified size in terms of approximate number of cells per cluster, one or more clonal cell clusters may be selected for testing to confirm that the desired transfection event has taken place. A portion or subset of the cells within the one or more selected clusters are detached, e.g., using a laser detachment technique, and removed from the device, e.g., using an intermediate cell removal port (indicated as the circle at the right-hand end of the device shown in FIG. 1) that is in fluid communication with an outlet of the cell selection compartment and in fluid communication with an inlet of the cell expansion compartment. The subset of cells that have been detached and removed from the device may be subjected to, e.g., nucleic acid sequencing, to confirm that the desired transfection event (e.g., a CRISPR edit) has taken place.

Based on the results of the testing, one or more of the selected and tested clonal cell clusters may be retained for further expansion, while all non-selected and/or unwanted cells or clonal cell clusters are destroyed, e.g., using a laser photoablation technique.

Following the destruction of non-selected and/or unwanted cells, the selected cells may be detached from their growth surface (e.g., using trypsin or by performing laser-based photodetachment) and transferred to the cell expansion compartment, where they are subjected to several cycles of cell growth and division. Cell growth may be monitored using a variety of techniques, e.g., by imaging the growth surface and/or by using electrodes integrated into the cell expansion compartment to make electrical impedance measurements.

Once the cells have reached a specified surface density or level of confluence, the clonal cell population may be detached from the growth surface in the cell expansion compartment, e.g., using trypsin, and harvested by removing them through the fluid outlet located at the lower left of the device illustrated in FIG. 1.

Although not explicitly shown in FIG. 1, in many instances the disclosed devices (or cartridges) may comprise an integrated cell culture (or cell growth) medium reservoir that supplies the cells growing in the cell selection and/or cell expansion compartments with fresh medium, and an integrated waste reservoir that stores the spent medium.

### Example 2—Preparation of Clonal Population of Cells in Cartridges Using Photoablation and Photodetachment

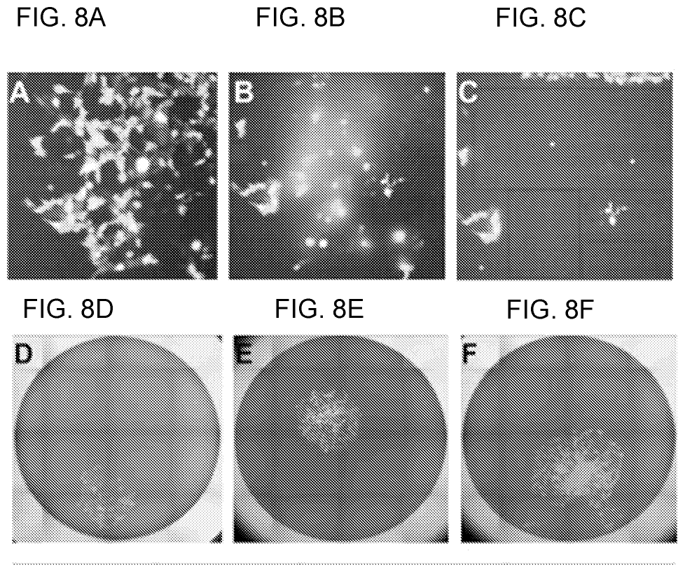

FIGS. 8A-8F show clonal isolation of cells using photoablation and photodetachment in cartridges described herein. FIG. 8A shows a mixed populations of HEK293-GFP and RFP transfected cells are after attachment on cartridges described herein. In FIG. 8B, mixed populations of HEK293-GFP and RFP cells are shown after laser ablation. FIG. 8C provides an illustration of mixed populations of HEK293-GFP and RFP cells after removal of dead cells by media flow, wherein the boxes indicate areas that were targeted for ablation. FIG. 8D shows clonal HEK293-RFP colonies after photodetaching them from the cartridges with no detectable cross contamination observed after export, while FIG. 8E shows clonal HEK-GFP colonies after photodetaching them from the cartridges with no detectable cross contamination observed after export. FIG. 8F depicts non-clonal cross contaminated colonies containing both populations of cells. The experiments depicted in FIGS. 8A-8F were performed according to the methods below.

Cell Culture:

HEK293-GFP (Creative Biogene, CSC-RR0040) and HEK293-RFP (Creative Biogene, CSC-RR0066) cells were cultured at 37 degrees 5% CO2 in Dulbecco's Modified Eagle's Medium (DMEM) supplemented with 10% Fetal Bovine Serum (FBS). Cells were dissociated using TryplExpress (Invitrogen) upon reaching 80% confluence for subculturing.

Cartridges described herein were aseptically assembled and loaded with 10 ml DMEM:10% FBS Media. Both HEK293-RFP and HEK293-GFP cells were simultaneously dissociated with TryplExpress, counted with a Nucleocounter (ChemoMetec), and pooled at a 4:1 GFP:RFP ratio. Pooled cells were diluted to 12,500/ml to achieve 2500 cells per device, then loaded into a cartridge with a P200 pipette through the media supply port leaving the export channel open to allow air to escape. After loading, the cartridge was closed and placed in an incubator with the following settings: 120 minute recovery, 240 minute pressurized degas, media changes every 48 hours.

Establishing Clones:

To establish clones, adjacent cells were laser ablated at 24 and 48 hours post-seeding to establish clonal populations with additional ablation at 5 days post seeding to enforce clonality. After each ablation the cell chamber was flushed with media to remove detached cells.

Exporting Clones:

8 days after seeding, individual clones were detached from the cartridges described herein using laser pulses and exported to a single well of a 96 well tissue culture plate. Clones were allowed to attach and grow for 4 days post-export, then imaged for GFP and RFP using a Celigo Imaging Cytometer (Nexcelom Bioscience). The frequency of homozygous RFP or homozygous GFP cells was determined by manual observation. The presence of a single contaminating cell was counted as fail.

### Example 3—Cross-Contamination Testing of Cartridges Comprising Valves

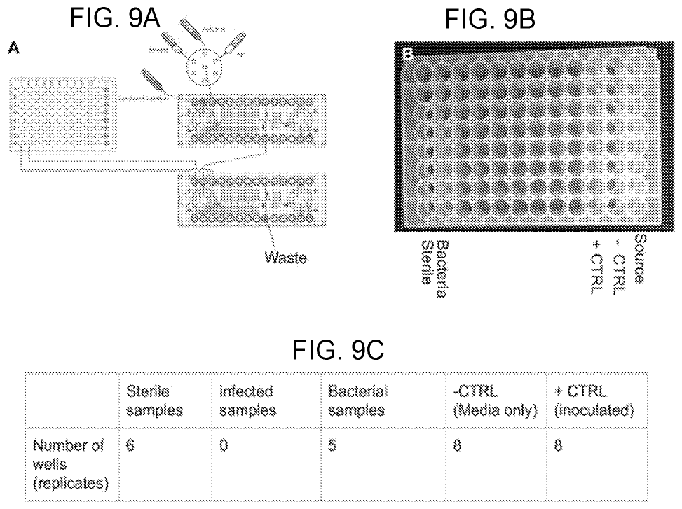

FIGS. 9A-9C depict testing cross-contamination via rotary valves of the cartridges described herein. FIG. 9A show the rotary valves and liquid paths of the cartridge. Inoculated bacteria and sterile media were transported between valves using a common liquid path. FIG. 9B: shows no cross contamination was observed in the cartridge comprising rotary valves. FIG. 9C: provides a tabular depiction of the cross-contamination testing results showing that no cross contamination was observed in the cartridge comprising rotary valves.

The experiments depicted in FIGS. 9A-9C were performed according to the methods below.

Bacterial Culture:

E. coli carrying an ampicillin resistance cassette in pBluescript plasmid were grown at 37 degrees with shaking in Lysogeny Broth (LB) with 100 ug/ml ampicillin (LB+Amp) for 24 hours before Sterilize in Place (SIP) testing.

Cross-Contamination/SIP Testing:

500 ul Bacterial culture was deposited into each well of column 12 of a deep-well polypropylene plate (destination plate). Bacterial culture was also loaded into a 10 ml syringe and attached to the initial rotary valve comprising cartridge. A 10 ml syringe was loaded LB+Amp and attached to the initial rotary valve comprising cartridge. Additionally LB+Amp was deposited into columns 10 and 11 of the destination plate. As a positive control for bacterial contamination, a P20 pipette tip was dipped bacterial culture in column 12, then dipped in media in column 10.

The following parameters were used for SIP testing:

1. Adjust rotate valves to allow bacterial solution to pass to column 1 of plate, apply pressure to 10 ml syringe, allow roughly 500 ul bacterial culture to accumulate in well on destination plate.
2. Rotate valves to connect cleaning and waste ports.
3. Flush with 70% isopropanol:water (to waste), flush with air (to waste).
4. Rotate second valve to open path to media wells on destination plate.
5. Apply pressure to media 10 ml syringe, allow roughly 500 ul media to accumulate in well on destination plate.
6. Rotate first and second valves to open bacterial path.
7. Repeat process for additional wells.

Place deepwell plate at 37 degrees for 24 hours. Determine bacterial inoculation by visual inspection for media turbidity.

### Example 4—Multi-Chamber Cartridge Embodiments

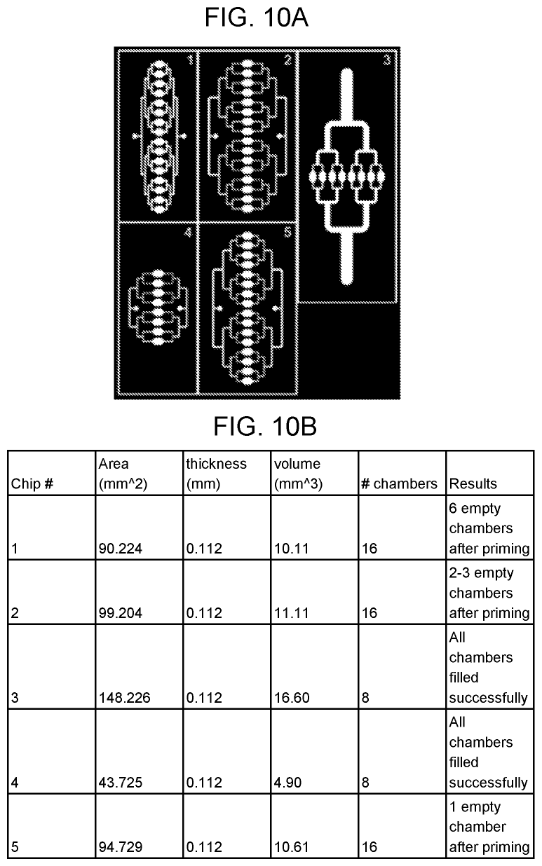

FIGS. 10A-10B show various multi-chamber embodiments of the cartridges described herein as well as testing results of these various embodiments. FIG. 10A shows multi-chamber embodiments of the cartridges described herein, while FIG. 10B: provides a table detailing the dimensions of the multi-chamber embodiments of the cartridges described herein and results of flow testing in said chambers. Multi-chamber cartridges were aseptically assembled and primed with DMEM:10% FBS Media administered by 200 ul pipette. Distribution of media across chip was determined visually.

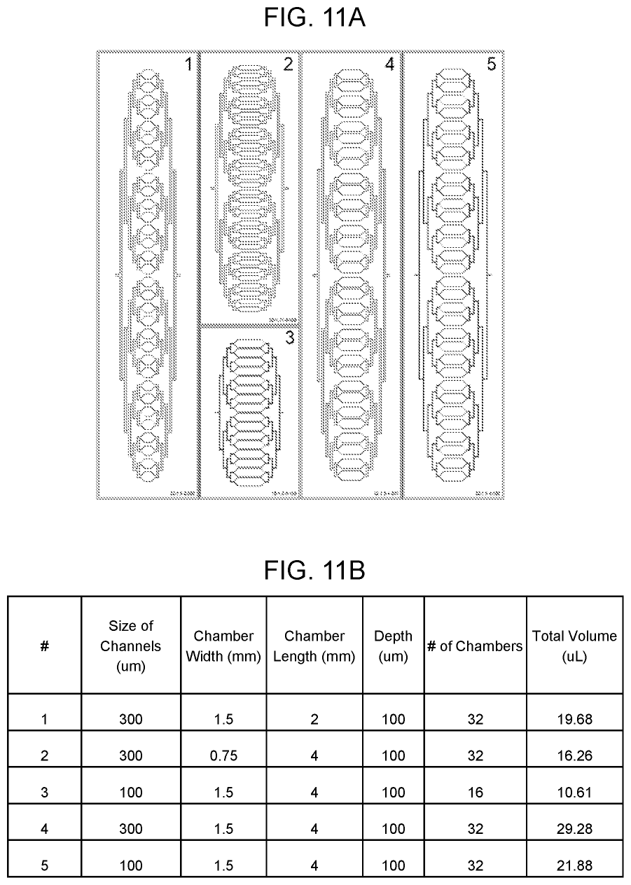

FIGS. 11A-11B show various multi-chamber embodiments of the cartridges described herein. FIG. 11A shows multi-chamber embodiments of the cartridges described herein and FIG. 11B provides a table detailing the dimensions of the multi-chamber cartridges described herein.

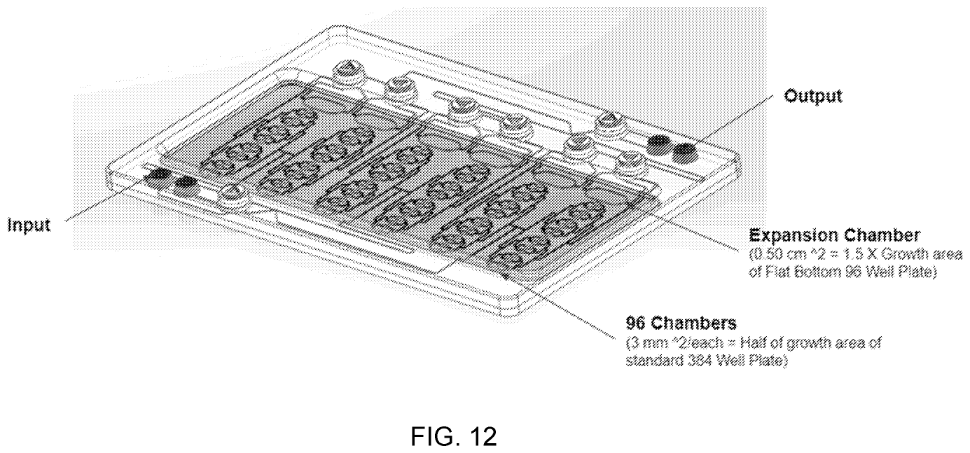

FIG. 12 shows an embodiment of the multi-chamber cartridges described herein featuring 96 parallel miniature cell culture chambers, 6 larger cell culture chambers and fluidic connections. The fluidic interfaces feature zero-unswept volume input/output connections with rotary valves for sealing the cartridge and sterilizing the input/output connections along with their associated flow paths. The input and output lines facilitate initial loading, feeding, waste removal, assays and export. Cells within selected miniature chambers could be exported to a larger chamber for expansion of the colony using a set of rotary valves.

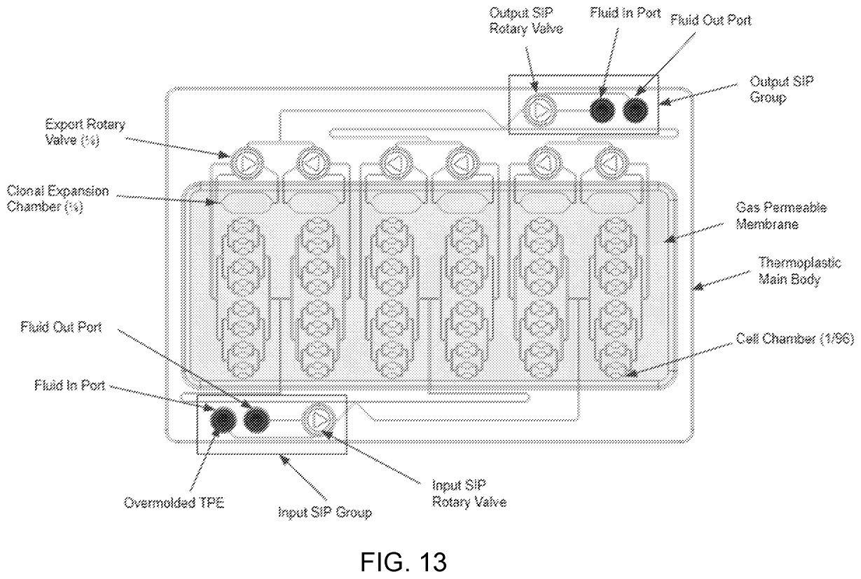

FIG. 13 shows an embodiment of the multi-chamber cartridges described herein featuring two sterilize in place (SIP) systems to facilitate flow in and out of the cartridge. This system contains two low-dead volume conical input/output ports composed of an overmolded thermoplastic elastomer material and a rotary shear type valve. This valve features a single internal channel with zero-unswept internal volume which can be rotated to connect the input port to the output port or the input port to the cell culture chambers. Sterilizing solutions may be introduced to the port and valve to sterilize them before switching the valve to the cell culture chambers. This SIP routine ensures that the fluidic interfaces and valve are sterile before making connection with the culture chambers.

### Example 5—Media Cartridge Embodiment

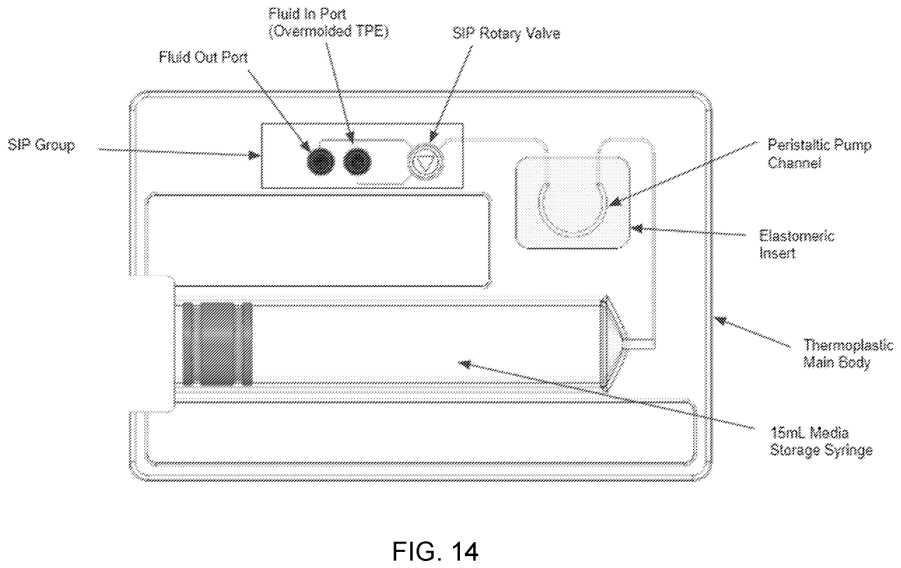

FIG. 14 shows an embodiment of a media cartridge featuring a media filled syringe, a pump interface for filling the syringe and a SIP system identical to the one in FIG. 13. The syringe features a free-moving piston which can be displaced as liquid enters or exits the chamber. The microfluidic passage downstream of the syringe features an elastic section routed in a partial arc. A peristaltic pump featuring one or more roller may be pressed against the elastic section and rotated in order to induce flow, either into or out of the syringe. Filling and dispensing from the syringe may be accomplished in a sterile way by means of the SIP system.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in any combination in practicing the invention. It is intended that the following claims define the scope of the invention and that methods and structures within the scope of these claims and their equivalents be covered thereby.

1. (canceled)

2. (canceled)

3. (canceled)

4. A system comprising:
a cartridge comprising at least one compartment configured for performing one or more of: cell transfection, cell selection and/or cell expansion, wherein the cartridge comprises an inlet configured for introduction of a cell sample and the at least one compartment comprises an optically-transparent wall.

5. The system of claim 4 further comprising a light source to facilitate performance of a photodetachment process and/or a photoablation process.

6. The system of claim 4, wherein the cartridge comprises at least one compartment for cell selection and at least one compartment for cell expansion.

7. The system of claim 4, wherein the cartridge comprises a plurality of compartments for cell selection and a plurality of compartments for cell expansion.

8. The system of claim 4, wherein the cartridge comprises at least four compartments for cell selection, at least eight compartments for cell selection, at least sixteen compartments for cell selection, at least thirty two compartments for cell selection, at least sixty four compartments for cell selection, or at least ninety six compartments for cell selection.

9. The system of claim 4, wherein the cartridge comprises at least four compartments for cell expansion, at least eight compartments for cell expansion, at least sixteen compartments for cell expansion, at least thirty two compartments for cell expansion, at least sixty four compartments for cell expansion, or at least ninety six compartments for cell expansion.

10. The system of claim 4, wherein the cartridge comprises at least one compartment for cell transfection.

11. The system of claim 4, wherein the cartridge does not comprise a compartment for cell transfection.

12. The system of claim 4, wherein the at least one compartment further comprises at least one of: (i) a second inlet configured for introduction of a transfection agent, (ii) a constricted flow path, and (iii) a pair of electrodes in electrical contact with and positioned on opposing surfaces of the at least one compartment.

13. The system of claim 12, wherein the pair of electrodes are fabricated from at least one of platinum, gold, silver, copper, zinc, aluminum, graphene, or indium tin oxide.

14. The system of claim 5, wherein the optically-transparent wall of the at least one compartment is transparent in the ultraviolet, visible, or near-infrared regions of the electromagnetic spectrum, or any combination thereof.

15. The system of claim 5, wherein the at least one compartment comprises a cell selection compartment, and wherein the cell selection compartment comprises a pattern of indentations on an inner surface and/or a pattern of a substrate on an inner surface.

16. The system of claim 5, wherein the at least one compartment comprises a cell expansion compartment, and wherein the cell expansion compartment comprises a pattern of indentations on an inner surface and/or a pattern of a substrate on an inner surface.

17. The system of claim 15, wherein the substrate is a protein substrate.

18. The system of claim 15, wherein the pattern of indentations and/or the pattern of a substrate are configured to prevent cell migration within the compartments.

19. The system of claim 4, wherein a volume of the at least one compartment is between about 1 microliter and about 10 milliliters.

20. The system of claim 4, wherein the optically-transparent wall of the at least one compartment is transparent in the ultraviolet, visible, or near-infrared regions of the electromagnetic spectrum, or any combination thereof.

21. The system of claim 4, wherein the optically-transparent wall of the at least one compartment is transparent in the range from about 1440 nm to about 1450 nm.

22. The system of claim 4, wherein a wall of the at least one compartment comprises a surface coating or surface treatment to facilitate attachment of adherent cells.

23. The system of claim 4, wherein a wall of the at least one compartment comprises a surface coating or surface treatment to facilitate attachment of suspension cells.

24. The system of claim 22, wherein the surface coating is selected from the group consisting of an α-poly-lysine coating, a collagen coating, a poly-1-ornithine, a fibronectin coating, a laminin coating, a Synthemax™ vitronectin coating, an iMatrix-511 recombinant laminin coating, and any combination thereof.

25. The system of claim 22, wherein the surface treatment comprises a plasma treatment, a UV treatment, an ozone treatment, or any combination thereof.

26. The system of claim 22, wherein the wall of the at least one compartment that comprises the surface coating or surface treatment is the optically-transparent wall.

27. The system of claim 4, wherein the at least one compartment comprises a chamber having no physical barriers, flow constrictions, or partitions positioned therein.

28. The system of claim 4, wherein a longest dimension of the at least one compartment is between about 1 centimeter and about 20 centimeters.

29. The system of claim 4, wherein a volume of the at least one compartment is between about 1 microliter and about 1 milliliter.

30. The system of claim 4, wherein the at least one compartment further comprises at least one optically-transparent wall.

31. The system of claim 30, wherein the optically-transparent wall is transparent in the ultraviolet, visible, or near-infrared regions of the electromagnetic spectrum, or any combination thereof.

32. The system of claim 4, wherein the at least one compartment further comprises at least one pair of electrodes configured for performing electrical impedance measurements.

33. The system of claim 4, wherein the at least one compartment comprises a compartment configured for storing a cell growth medium.

34. The system of claim 4, wherein the at least one compartment comprises a compartment configured for storing waste.

35. The system of claim 33, wherein the compartment further comprises a gas permeable membrane.

36. The system of claim 4, wherein the inlet of the at least one compartment is operably coupled to a source of a reagent that facilitates detachment of cells from a surface within the at least one compartment.

37. The system of claim 4, wherein the inlet of the at least one compartment is operably coupled to a source of a reagent that facilitates detachment of cells from a surface within the at least second compartment.

38. The system of claim 4, wherein the cartridge is fabricated from glass, fused-silica, silicon, polydimethylsiloxane (PDMS), polymethylmethacrylate (PMMA), polycarbonate (PC), polypropylene (PP), polyethylene (PE), high density polyethylene (HDPE), polyimide (PI), cyclic olefin polymers (COP), cyclic olefin copolymers (COC), polyethylene terephthalate (PET), polystyrene (PS), epoxy resin, ceramic, metal, or any combination thereof.

39. The system of claim 4, wherein the at least one compartment comprises a first compartment, a second compartment, and a third compartment, wherein the second compartment comprises an outlet and wherein the third compartment comprises a cell removal port and an inlet, wherein the outlet of the second compartment is operably coupled to the cell removal port and the inlet of the third compartment using a first valve.

40. The system of claim 39, wherein the at least one compartment further comprises a fourth compartment comprising an outlet, wherein the inlet of the third compartment is operably coupled to the outlet of the second compartment and the outlet of the fourth compartment using a second valve.

41. The system of or claim 40, wherein at least one of the first valve or the valve is a programmable three-way valve.

42. The system of claim 4, wherein the microfluidic cartridge has a footprint that complies with American National Standards Institute (ANSI) Standard Number SLAS 4-2004 (R2012).

43. The system of claim 4, wherein the cartridge has a footprint that is 127.76 mm±0.5 mm in length and 85.48 mm±0.5 mm in width.

44-142. (canceled)

143. The system of claim 4, wherein the at least one compartment of the cartridge comprises a first compartment, a second compartment, and a third compartment;
wherein the first compartment is configured for performing cell transfection and wherein the first compartment comprises a first inlet configured for introduction of a cell sample;
wherein the second compartment is configured for performing cell selection, wherein an inlet of the second compartment is operably coupled to an outlet of the first compartment, and wherein the second compartment further comprises at least one optically-transparent wall and an outlet that is operably coupled to an intermediate cell removal port; and
wherein the third compartment is configured for performing cell expansion, wherein an inlet of the third compartment is operably coupled to the outlet of the second compartment.

144. The system of claim 4, wherein the at least one compartment of the cartridge comprises a first compartment, a second compartment, and a third compartment;
wherein the first compartment is configured for performing cell transfection, wherein the first compartment comprises a first inlet configured for introduction of a cell sample;
wherein the second compartment is configured for performing cell selection, wherein an inlet of the second compartment is operably coupled to an outlet of the first compartment, and wherein the second compartment further comprises at least one optically-transparent wall; and
wherein the third compartment is configured for performing cell expansion, wherein the third compartment comprises at least one pair of electrodes configured for performing electrical impedance measurements, and wherein an inlet of the third compartment is operably coupled to the outlet of the second compartment.

145. The system of claim 4, wherein the at least one compartment of the cartridge comprises a first compartment, a second compartment, and a third compartment;
wherein the first compartment is configured for performing cell transfection, wherein the first compartment comprises a first inlet configured for introduction of a cell sample;
wherein the second compartment is configured for performing cell selection, wherein an inlet of the second compartment is operably coupled to an outlet of the first compartment, and wherein the second compartment further comprises at least one optically-transparent wall that is operably coupled to a source of laser light for performing photoablation and photodetachment; and
wherein the third compartment is configured for performing cell expansion, wherein an inlet of the third compartment is operably coupled to the outlet of the second fluid compartment.
